(19) United States
(12) Patent Application Publication
(10) Pub. No.: US 2026/0209218 A1
(43) Pub. Date: Jul. 23, 2026

(54) 3-PHENYLQUINAZOLINONES AS NOVEL ANTI-CANCER THERAPY

(71) Applicant: HELMHOLTZ ZENTRUM MÜNCHEN - DEUTSCHES FORSCHUNGSZENTRUM FÜR GESUNDHEIT UND UMWELT (GMBH), Neuherberg (DE)

(72) Inventors: Marcus CONRAD, Neuherberg (DE); Toshitaka NAKAMURA, Neuherberg (DE); Bettina PRONETH, Neuherberg (DE); Peter C. SENNHENN, Neuherberg (DE)

(73) Assignee: HELMHOLTZ ZENTRUM MÜNCHEN - DEUTSCHES FORSCHUNGSZENTRUM FÜR GESUNDHEIT UND UMWELT (GMBH), Neuherberg (DE)

(21) Appl. No.: 19/133,590

(22) PCT Filed: Nov. 30, 2023
(86) PCT No.: PCT/EP2023/083768

§ 371 Date: May 28, 2025

(30) Foreign Application Priority Data
Nov. 30, 2022 (EP) 22210489.5
Jun. 28, 2023 (EP) 23182217.2

(51) Int. Cl. C07D 471/04; A61K 31/517; A61K 31/519; A61P 35/00; C07D 239/91; C07D 401/04; C07D 401/12; C07D 403/04; C07D 405/12; C07D 487/04; C07D 491/056; C07D 495/04

(52) U.S. Cl. CPC C07D 471/04; A61K 31/517; A61K 31/519; A61P 35/00; C07D 239/91; C07D 401/04; C07D 401/12; C07D 403/04; C07D 405/12; C07D 487/04; C07D 491/056; C07D 495/04

(57) ABSTRACT

The present invention relates to novel 3-Phenylquinazolinones in particular for the use in the treatment of cancer.

### TECHNICAL FIELD OF THE INVENTION

The present invention relates to novel 3-Phenylquinazolinones in particular for the use in the treatment of cancer.

### BACKGROUND ART

Ferroptosis is a form of regulated necrotic cell death that is tightly controlled by glutathione peroxidase 4 (GPX4)¹⁻³ This pathway continues to emerge as an important player in a variety of physiological and pathological conditions including antiviral immunity, neurodegeneration, ischaemia/reperfusion injury and tumor suppression⁴⁻⁷. At present, many efforts have been made to characterize genes that are essential for this form of cell death and to develop novel ferroptosis inhibitors^{2,8}. While some ferroptosis inducing compounds targeting different nodes of the ferroptosis regulating cascade have been described in the past^{4,9}, most of them fail to confer strong in vivo ferroptosis inducing activity. This is largely because several of these checkpoints can be bypassed in vivo.

At present, no efficient small molecule-based ferroptosis inducing anti-cancer strategy exists. In addition, targeting the cyst(e)ine/glutathione/GPX4 axis has been reported by using genetically engineered cyst(e)inase, next generation system Xc⁻ inhibitors (e.g., piperazine erastin, imidaketazole erastin) and next generation GPX4 inhibitors (e.g., ML210, diacylfluroxanes, masked nitrile-oxide electrophiles). Yet, none of them has proven to be efficacious to efficiently inhibit tumor growth in vivo¹⁰⁻¹³ Moreover, many of the known ferroptosis inducing compounds do elicit ferroptosis in cell culture, yet they fail to do so in vivo. This is largely owed to the fact that they are only poorly bioavailable or target redundant nodes in the ferroptosis regulatory network⁶. Moreover, some of the known targets (e.g., GPX4) are essential for normal physiology as demonstrated by multiple genetic studies⁷, thereby targeting these nodes might be potentially associated with severe toxicity issues if pharmacologically inhibited. By contrast, FSP1 knockout mice are fully viable^{14,15} which might allow for a significant therapeutic window for newly developed FSP1 inhibitors. In fact, the first described inhibitor of FSP1 (i.e., iFSP1; 1-amino-3-(4-methylphenyl)-pyrido[1,2-a]benzimidazole-2,4-dicarbonitrile) efficiently sensitizes ferroptosis-resistant tumor cells towards ferroptosis¹⁶⁻¹⁸ although this class of compounds is of little use for further development as future drugs.

Thus, there is a need for further small molecule-based ferroptosis inducing cancer therapy.

### SUMMARY OF THE INVENTION

The invention is directed to a compound according to formula (I) or (II), preferably formula (I) for use in the treatment of cancer,

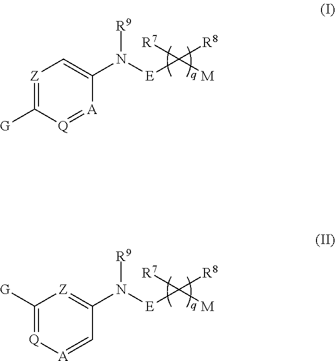

wherein G is selected from the group consisting of

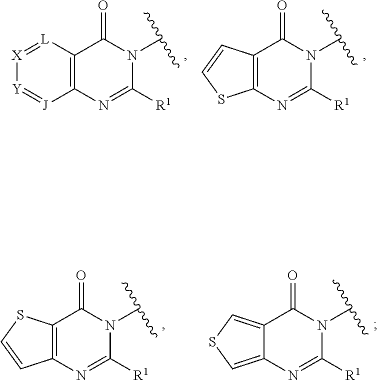

M is selected from the group consisting of

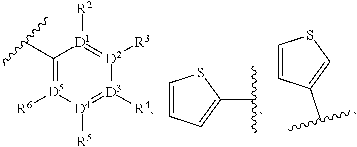

Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
L is selected from the group consisting of CH, N, C—F, C—Cl, and C—(C₁-C₆)alkyl, preferably CH;
X is selected from the group consisting of CH, N, C—F, C—Cl, and C—(C₁-C₆)alkyl, preferably CH;
Y is selected from the group consisting of CH, N, C—F, C—Cl, and C—(C₁-C₆)alkyl, preferably CH;
X and Y may be part of ring according to formula (III):

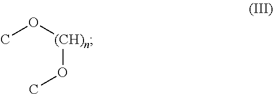

n is an integer from 1 to 3;
J is selected from the group consisting of CH, N, C—F, C—Cl, C—(C₁-C₆)alkyl, preferably CH;
D¹, D², D³, D4, D⁵, are independently selected from the group consisting of C and N;
R¹ is selected from the group consisting of —(C₁-C₆)alkyl, —(C₃-C₆)cycloalkyl, —CH₂OH—CH₂F, —CHF₂, and —CF₃, preferably —(C₁-C₆)alkyl;
R² is selected from the group consisting of H, —(C₁-C₆)alkyl, —CF₃, —Cl, and —F, preferably H;
R³ is selected from the group consisting of H, —(C₁-C₆)alkyl, —CF₃, —O(C₁-C₆)alkyl, —Cl, and —F, preferably —O(C₁-C₆)alkyl;
R⁴ is selected from the group consisting of H, —(C₁-C₆)alkyl, —CF₃, —O(C₁-C₆)alkyl, —Cl, and —F, preferably —O(C₁-C₆)alkyl;
R⁵ is selected from the group consisting of H, —(C₁-C₆)alkyl, —CF₃, —O(C₁-C₆)alkyl, —Cl, and —F, preferably —O(C₁-C₆)alkyl;
R⁶ is selected from the group consisting of H, —(C₁-C₆)alkyl, —CF₃, —Cl, and —F, preferably H;
two groups of R², R³, R⁴, R⁵ and R⁶ in vicinal position may be part of a ring selected from

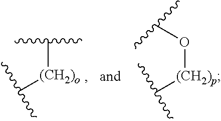

is an integer between 1 and 4;
p is an integer between 1 and 4;
q is 1 or 0;
R⁷ and R⁸ are independently selected from the group consisting of H, and —(C₁-C₆)alkyl;
R⁷ and R⁸ may form a 3 to 5 membered cycloalkyl ring;
R⁹ is selected from the group consisting of H, and —(C₁-C₆)alkyl or
a pharmaceutically acceptable salt thereof.

The invention is further directed to a pharmaceutical composition comprising a compound as defined as described above and at least one pharmaceutically acceptable carrier for use in the treatment of cancer.

### BRIEF DESCRIPTION OF THE FIGURES

FIG. 1 illustrates that compound 1 (alias icFSP1) efficiently induces ferroptosis in Pfa1 Gpx4^{KO} cells overexpressing FSP1 (A), as well as in HT-1080 wild type cells (a human fibrosarcoma cell line) (B) using AquaBluer as a measure of cell death. This form of cell death can be rescued by the ferroptosis inhibitor liproxstatin-1 (Lip-1). (C) Cell death was also determined using lactate dehydrogenase (LDH) release after treating Pfa1 Gpx4^{KO} cells overexpressing HA-tagged hFSP1 with DMSO (as control), 2.5 μM icFSP1, or 2.5 μM icFSP1+0.5 μM Lip-1 for 24 h. (D, E) Lipid peroxidation was evaluated by C11-BODIPY 581/591 staining after treating Gpx4^{KO} cells stable overexpressing HA-tagged hFSP1 with DMSO, 2.5 μM icFSP1+0.5 μM Lip-1 for 3 h. Representative plots of one of three independent experiments (D) and quantified median values of three independent experiments (E) are shown for the BODIPYₒₓ/BODIPYᵣₑ ratio of oxidized to reduced BODIPY Data represent the mean±s.e.m. of three independent of experiments (C, D, E). (F) Lipid peroxidation profiles measured by liquid chromatography and tandem mass spectrometry (LC-MS/MS) after treatment of Gpx4^{KO} Pfa1 cells stably overexpressing HA-tagged hFSP1 with DMSO, 5 μM icFSP1, or 5 μM+0.5 μM Lip-1 for 5 h. The heat map shows three technical replicates from one of two independent experiments.

FIG. 2 (A) shows the in vitro inhibitory activity of compound 1 (icFSP1) towards recombinant FSP1 enzyme activity. This assay shows the difference between the inhibition of resazurin reduction at different concentrations of the known direct inhibitor compound iFSP1¹⁶ as compared to icFSP1. (B) Representative time-lapse fluorescence images acquired immediately after treatment of Gpx4^{WT} Pfa1 cells stably overexpressing hFSP1-EGFP-Strep with 2.5 μM icFSP1. Scale bars, 10 μm. Representative results showing subcellular relocation of FSP1 upon treatment from one of three independent experiments. (C) The number of condensates induced by icFSP1 per cell quantified from time-lapse images at different time points (0, 60, 120, 180 and 240 min) after treatment obtained from one of two independent experiments. Dots represent each cell and n corresponds to cell number (n=129, 124, 130, 130 and 134 [left to right]). P values were calculated by one-way ANOVA followed by Dunnett's multiple-comparison test. (D) Representative time-lapse fluorescence images before and after treatment of Gpx4^{KO} Pfa1 cells stably overexpressing hFSP1-mTagBFP with 2.5 μM icFSP1 in FluoroBrite DMEM containing PI (0.2 μg/ml). Cells were pre-stained with 5 μM Liperfluo for 1 h. Scale bars, 10 μm. Representative results from three independent experiments.

FIG. 3 illustrates the in vivo efficacy of icFSP1 (compound 1) on tumor growth inhibition. (A) Gpx4 KO/Fsp1KO double mutant murine melanoma cells (B16F10) cells reconstituted with human FSP1 expression were subcutaneously implanted into C57BL6/J mice. From day 6 post implantation, treatment with either vehicle (control group n=6) or icFSP1 (n=7) was started. Tumor volume (left) and tumor weight (right) were measured daily or at the end of the experiment. (B) GPX4 KO human melanoma cells (A375) cells were subcutaneously implanted into nude mice. From day 3 post implantation, treatment with either vehicle (control group n=7) or icFSP1 (n=7) was started. Compound treatment inhibited tumor growth in both human and mouse tumors significantly. These results thus suggest that FSP1 inhibitors synergize with ferroptosis inducing agents (as exemplified by the knockout of the key ferroptosis regulator GPX4) to potentiate the cell death response. P value was calculated by two-way ANOVA followed by Bonferroni's multiple comparison test or unpaired t-test.

FIG. 4: FSP1 condensates are liquid droplets. (A) Representative time-lapse fluorescence images before and after treatment of Gpx4^{WT} Pfa1 cells stably overexpressing hFSP1-EGFP-Strep with 2.5 μM icFSP1 (compound 1). Arrowheads indicate fusion events of individual condensates (left). Reversibility of hFSP1 condensates (right). After treatment of cells with icFSP1 for 240 min, the medium was replaced with fresh medium without icFSP1 and recordings were restarted. Scale bars, 10 μm or 2 μm for zoomed-in images. Representative results from one of three independent experiments. (B) Fluorescence recovery after photobleaching (FRAP) assays after treatment of hFSP1-EGFP-Strep-overexpressing Gpx4^{WT} Pfa1 cells with 2.5 μM icFSP1 for 120 min. Greyscale images corresponding to representative FRAP images immediately before and after photobleaching (top-left). Lookup Table (LUT) images showing enlarged views of the areas in red rectangles in the upper FRAP images (bottom-left). Quantified FRAP rate of each condensate (right). Data represent the mean±s.d. of five condensates from the left images. Scale bars, 10 μm. Representative results from one of three independent experiments.

FIG. 5: Distinct structural features of FSP1 are required for phase separation. (A) Representative images of Pfa1 cells overexpressing hFSP1-EGFP-Strep mutants treated with 2.5 μM icFSP1 (compound 1). Scale bars, 10 μm. (B) Representative images of Pfa1 cells overexpressing WT hFSP1-EGFP-Strep or the S187C, L217R or Q319K variant treated with 2.5 μM icFSP1. Scale bars, 10 μm. Data is shown as the mean±s.d. of three different fields from one of three independent experiments (A, B). Statistical analysis was performed by one-way ANOVA followed by Dunnett's multiple-comparison test (B). (C) Cell viability measured after treatment of Gpx4^{KO} Pfa1 cells overexpressing WT hFSP1 or the S187C, L217R or Q319K variant with icFSP1 for 24 h. Data represent the mean±s.d. of n=3 wells from one of four independent experiments. (D) Gpx4 KO/Fsp1KO double mutant B16F10 cells reconstituted with human WT or Q319K FSP1 expression were subcutaneously implanted into C57BL6/J mice. At the end of the experiment, the tumors were dissected and stained with anti-HA (hFSP1) to visualize FSP1. Representative zoomed-in images are shown from one of three different tumor samples from one of two independent experiments (D). Arrowheads indicate FSP1 condensates (D). Scale bars, 10 μm (E).

FIG. 6: Synergistic effects of icFSP1 (compound 1) with ferroptosis inducers in a variety of human cancer cells. (A) Cell viability was measured after treating HT-1080, A375, 786-0, MDA-MB-436 and H460 cells with different ferroptosis inducers (RSL3, ML210, erastin, FIN56 and FINO2 for 48 h, BSO for 72 h). Heatmaps represent one out of 2 independent experiments. 0.5 μM Lip-1 was used as control. (B) Cell viability was measured after treating Pfa1 Gpx4^{WT} cells stably overexpressing hFSP1-HA, HT-1080, HT-29 and THP-1 cells with icFSP1 (compound 1) and respective cell death inhibitors (ferroptosis: liproxstain-1 (Lip-1), ferrostatin-1 (Fer-1), and deferoxamine (DFO), apoptosis (zVAD-FMK), necroptosis (Nec-1s), pyroptosis (MCC950) and parthanatos (olaparib) or inducers (staurosporine for apoptosis, TNFα+smac mimetic (S)+z-VAD-FMK (Z) for necroptosis, and nigericin for pyroptosis) for 4 h (in case of pyroptosis) or 24 h (others). Heatmaps represent one out of 2 independent experiments. 0.5 μM Lip-1, 30 μM z-VAD-FMK, 10 μM Nec-1s and 10 μM MCC950 were used as positive controls for each mode of cell death. (C). Cell viability in HT-1080 cells treated with iFSP1 or icFSP1 and 0.5 μM Lip-1 for 72 h. (D) Cell viability in HEK293T cells treated with iFSP1 or icFSP1 and 0.5 μM Lip-1 for 72 h. (E) Cell viability in human PBMC cells treated with iFSP1 or icFSP1 for 24 h. (F) Cell viability in FSP1 WT or FSP1 KO MDA-MB-436, 786-0, A375, H460 cells treated with 5 μM iFSP1 or icFSP1 for 48 h. Data represent the mean±SD of 3 wells of a 96 well or 384 well plates from one out of 2 independent experiments (A-F) or a single experiment (E). Two-way ANOVA followed by Tukey's multiple comparison tests (E).

FIG. 7: FSP1 forms viscoelastic material. (A) Fluorescence recovery after photobleaching (FRAP) assay after treating Pfa1 Gpx4^{WT} cells stably overexpressing hFSP1-EGFP-Strep with 2.5 μM icFSP1 (compound 1) for 240 min. Greyscale images show representative FRAP images right before and at indicated time points after photo-bleaching. Lookup Table (LUT) images show enlarged red rectangle areas of upper FRAP images. Scale bars, 5 μm. Representative results from one out of 2 independent experiments. (B) Quantified FRAP rate of each condensate. Data represent the mean±SD of 4 condensates Representative results from one out of 2 independent experiments are shown. (C) Absorbance of 600 nm was measured for different concentrations of PEG and icFSP1, non-myr-FSP1. Data represent the mean±SD from 4 wells of 384 well plates from one out of 2 independent experiments.

FIG. 8: Myristoylation is required for FSP1 condensations. (A) Confocal microscopy images of hFSP1-EGFP-Strep overexpressing Pfa1 Gpx4^{WT} cells after pre-treating with or without 0.1 μM IMP-1088 for 24 h and subsequently treating with or without 2.5 μM icFSP1 (compound 1) for the indicated time. Scale bars, 10 μm. Representative results from one out of 2 independent experiments. (B) Time-lapse fluorescent images of Pfa1 Gpx4^{WT} cells stably overexpressing hFSP1-EGFP-Strep; hFSP1-mTagBFP or hFSP1-G2A-EGFP-Strep; hFSP1-mTagBFP before and immediately after treatment with 2.5 μM icFSP1 for the indicated times. Scale bars, 10 μm. Representative results are from one out of 2 independent experiments.

FIG. 9: Mutational analysis of human FSP1 resistant to icFSP1 (compound 1). Saturation transfer difference (STD) spectra of WT hFSP1 or its mutants S187C, L217R and Q319K show binding of icFSP1 (bottom to top). Top spectrum shows a 1D 1H reference spectrum of icFSP1.

FIG. 10: Targeting of FSP1 by icFSP1 (compound 1) as potential anti-cancer therapy. (A) Pharmacokinetic (PK) parameters of icFSP1 and iFSP1. Plasma concentration was measured after single i.p. administration (10 mg/kg). Data represent mean±SD from 3 mice of one experiment. (B) Summary of microsomal stability analysis of icFSP1 and iFSP1. (C) Body weight of tumor-baring mice during the treatment of mice with icFSP1 (50 mg/kg i.p. twice a day, n=7) and vehicle (n=6) as a control. (D) Cell viability was measured after treating B16F10 Gpx4 KO/Fsp1 KO cells stably overexpressing FSP1-WT or Q319K mutants with icFSP1 for 48 h. Data represent the mean±SD of 3 wells from one out of 2 independent experiments. (E) icFSP1 inhibits tumor growth of hFSP1 WT but not of FSP1-Q319K expressing cells in vivo. B16F10 Gpx4 KO/Fsp1 KO cells stably expressing hFSP1 WT or Q319K were subcutaneously implanted into C57BL6/J mice (n=33, in total). Treatment with vehicle (n=10 for WT and 8 for Q319K) or icFSP1 (50 mg/kg i.p. twice per day, n=8 for WT and 7 for Q319K) was started from day 6 after randomization. Data represent the mean±SEM from one out of 2 experiments. P values were calculated by two-way ANOVA followed by Tukey's multiple comparison test.

FIG. 11: Targeting of FSP1 by icFSP1 (compound 1) as potential anti-cancer therapy using human cells. (A) At the end of the in vivo mouse studies, tumors were dissected, cryosectioned and stained with anti-FSP1 (14D7) to visualize hFSP1 and with anti-4-HNE to visualize a lipid peroxidation breakdown product. Representative confocal microscopy images of 3 different samples from a single experiment are shown. (B) Cell viability was measured after treating H460 WT and GPX4 KO cells with icFSP1 for 48 h. Data represent the mean±SD of 3 wells from one out of 2 independent experiments. (C) icFSP1 inhibits tumor growth in vivo. H460 GPX4 KO cells (a human lung cancer cell line) were subcutaneously implanted into the flanks of Athymic Nude mice. After tumors became palpable (day 8), mice were randomized and treatment was started with icFSP1 (50 mg/kg i.p. twice per day, n=7) or vehicle (n=7). Data represent the mean±SD from one experiment. P value was calculated by two-way ANOVA followed by Sidak's multiple comparison tests.

FIG. 12: FSP1 is a potential target in multiple cancer cells. Cell viability in lymphoma (SUDHL5, SUDHL6, DOHH2, OCI-Ly19) cells treated with icFSP1 (compound 1) in the presence or absence of 0.5 μM Lip-1 for 72 h. Data represent the mean±SD of 3 wells from one out of 2 independent experiments. P values were calculated by two-way ANOVA followed by Tukey's multiple comparison tests.

FIG. 13: Plasma concentration-time curve of compound 13 in male mice following IP (10 mg/kg) administration.

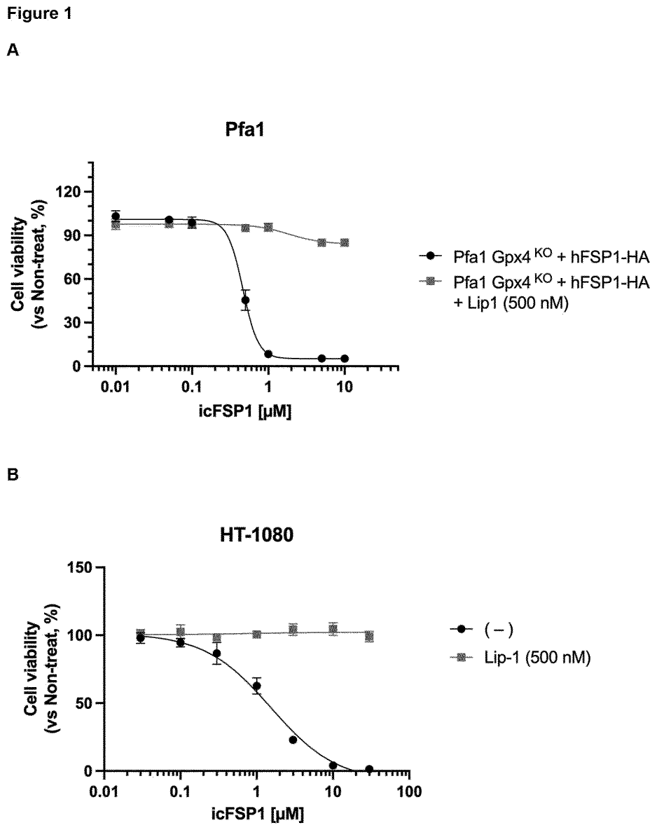

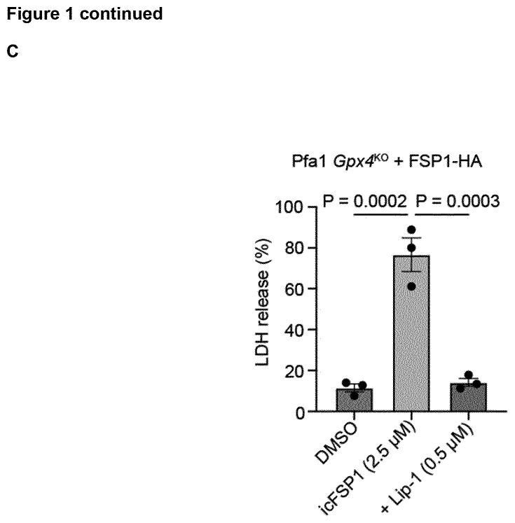

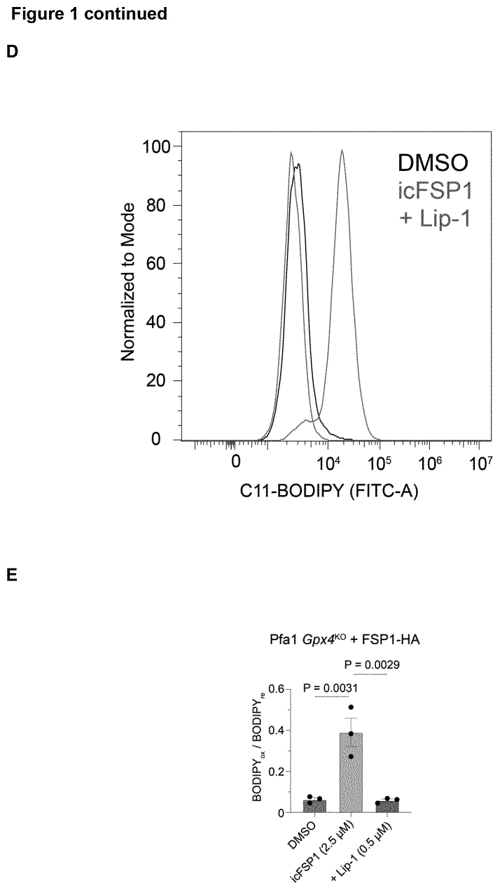

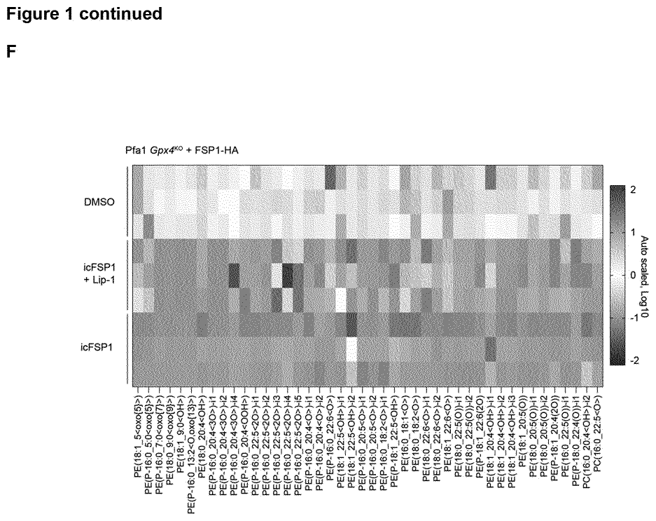

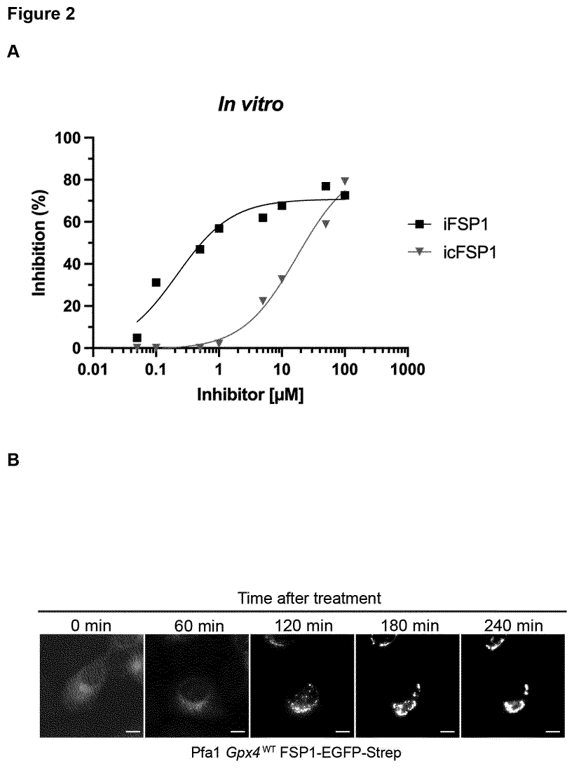

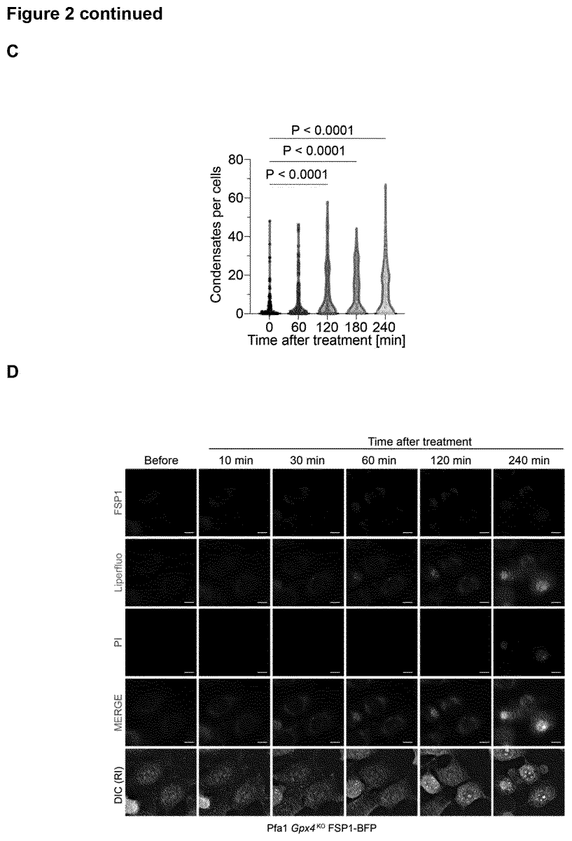

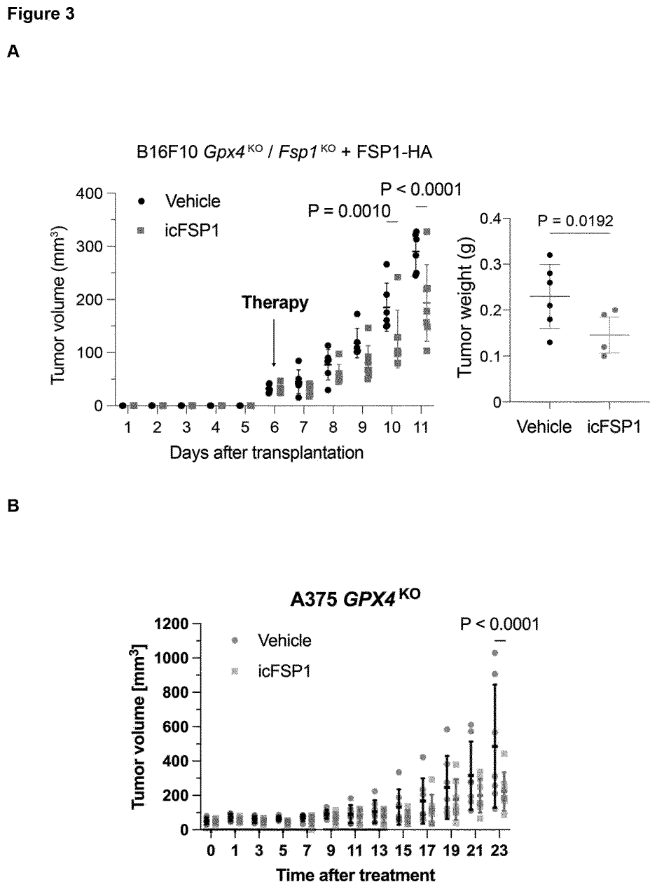

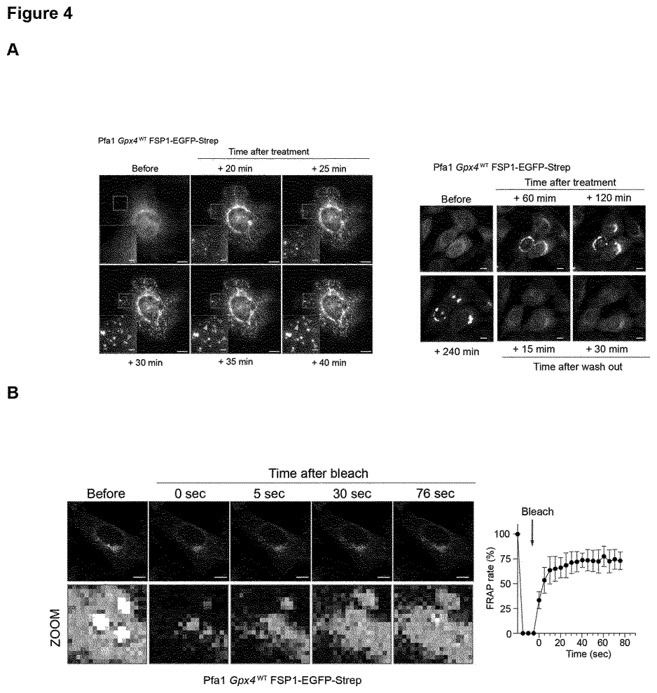

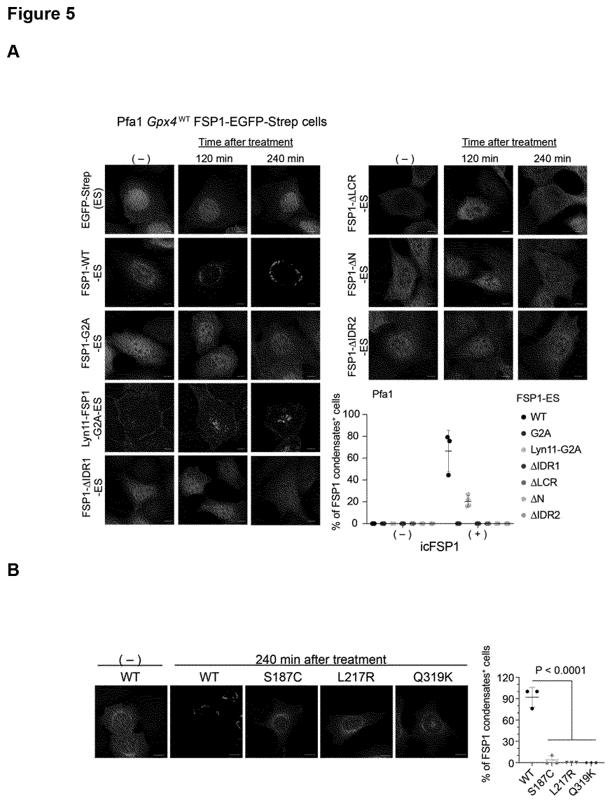

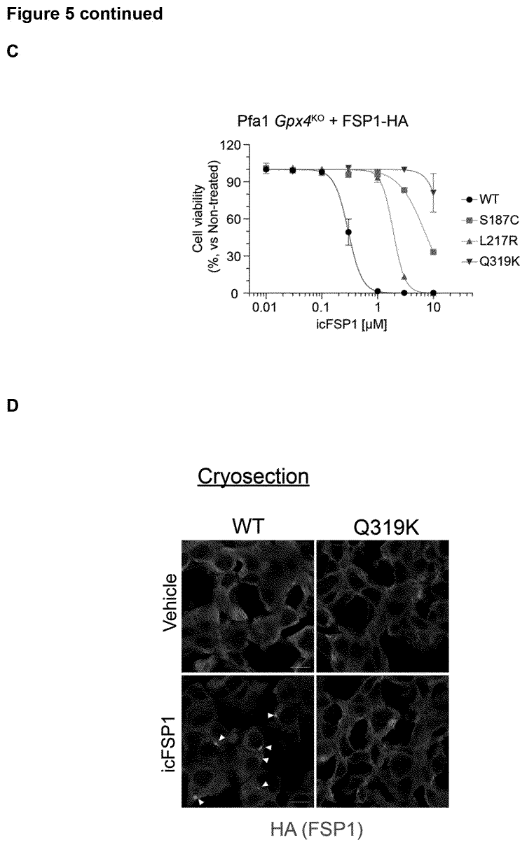

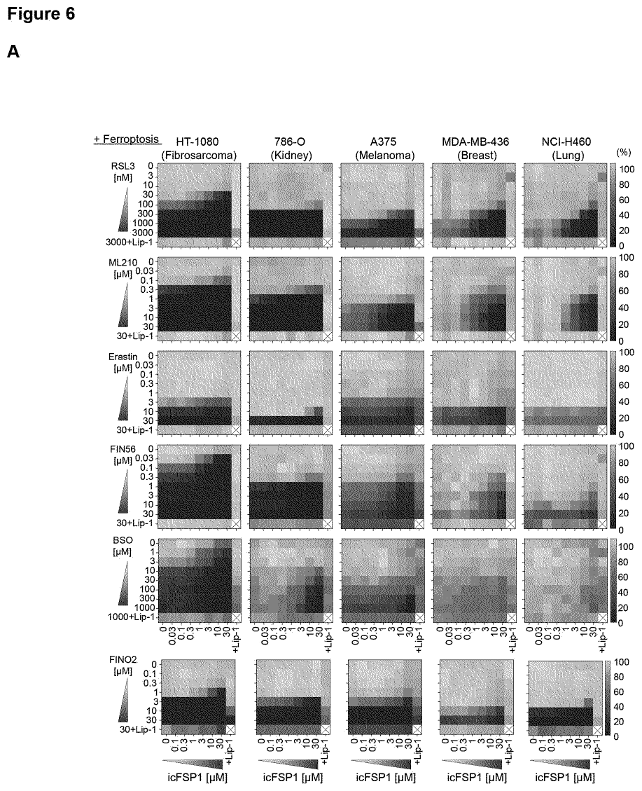

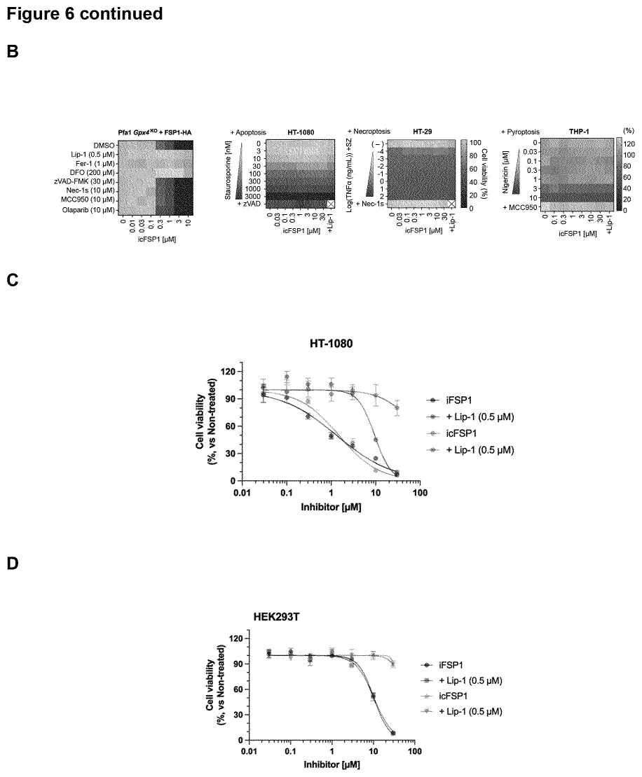

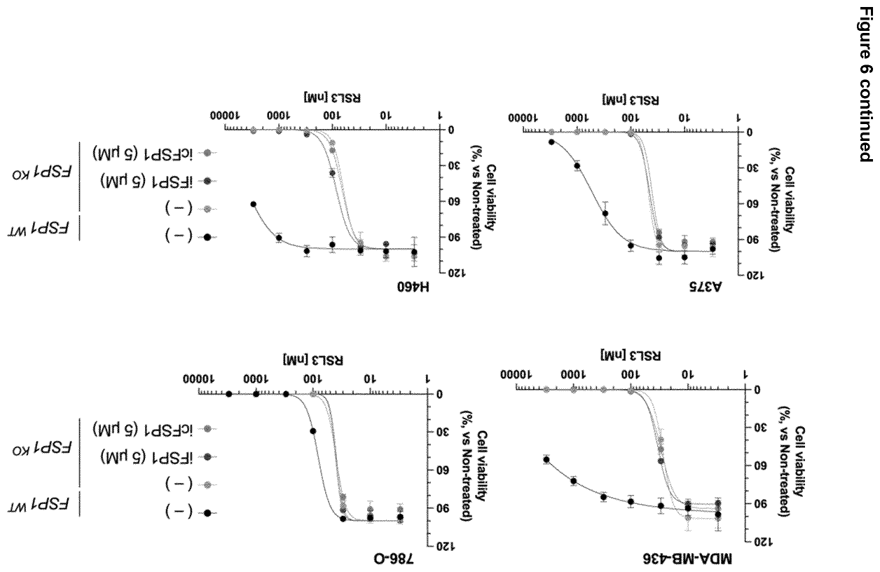

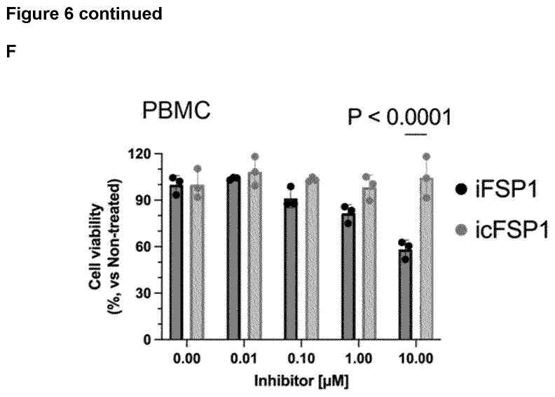

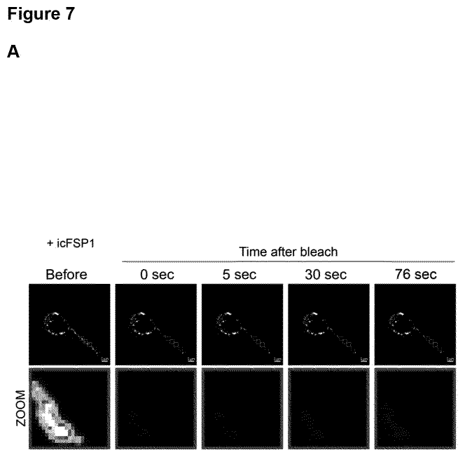

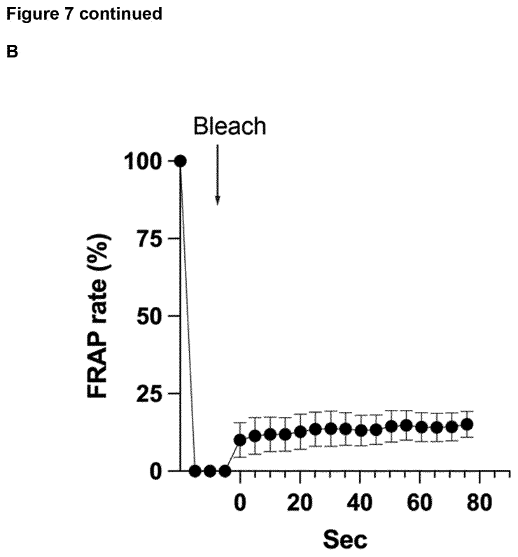

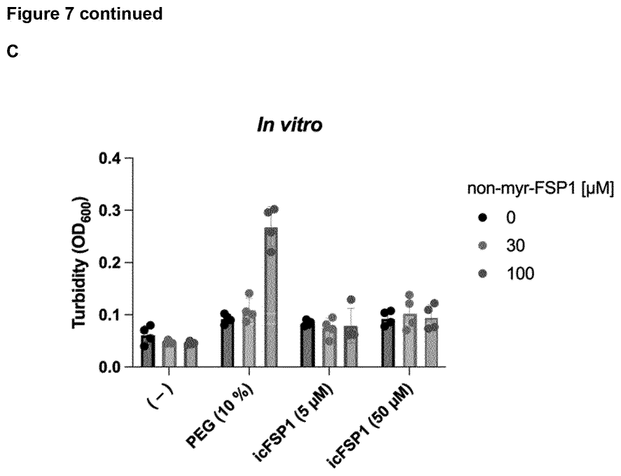

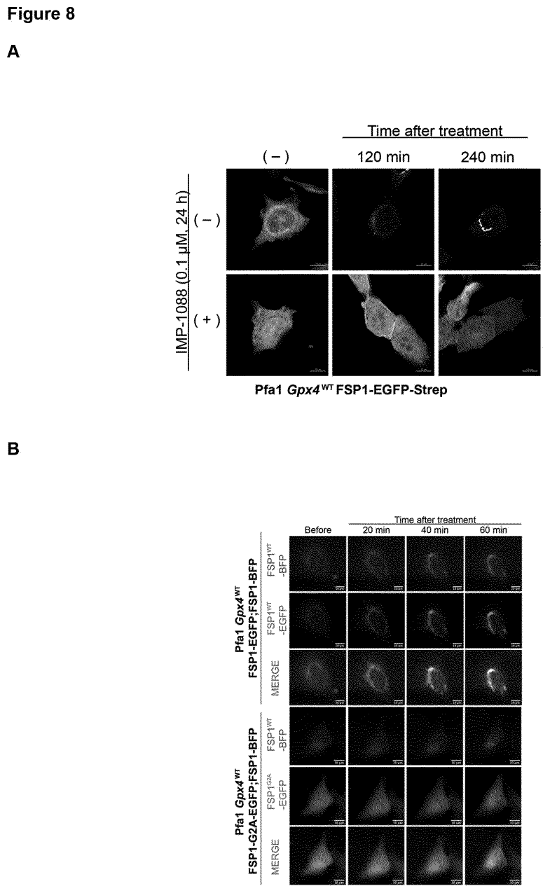

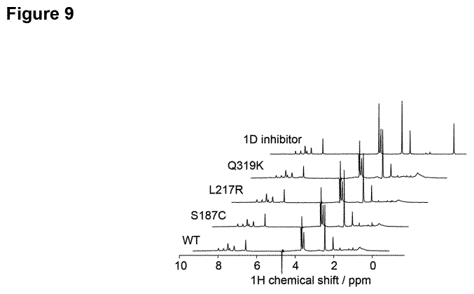

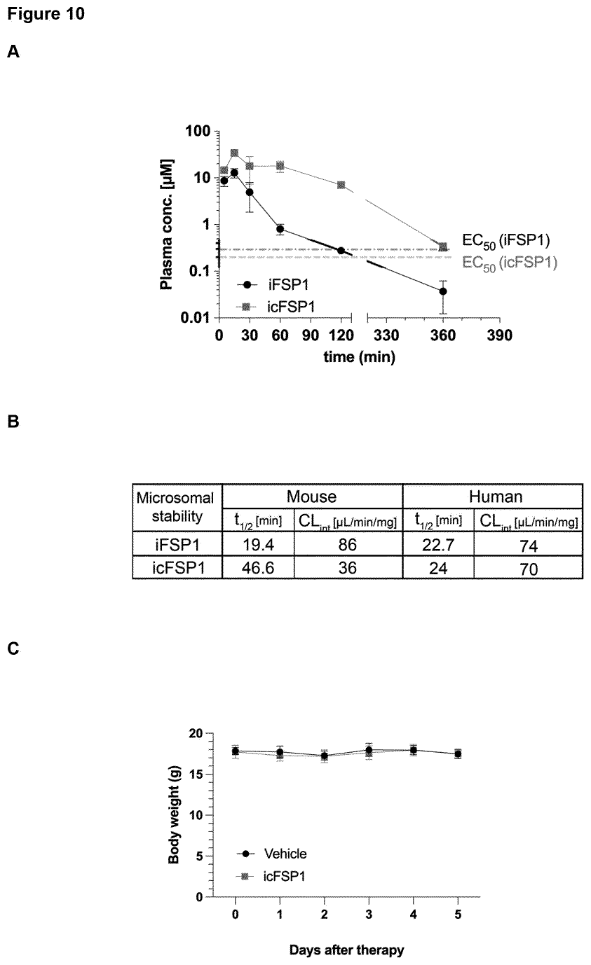

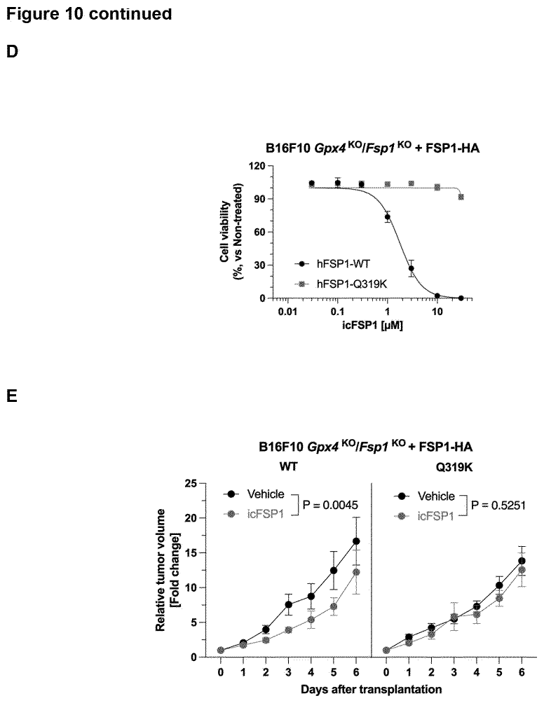

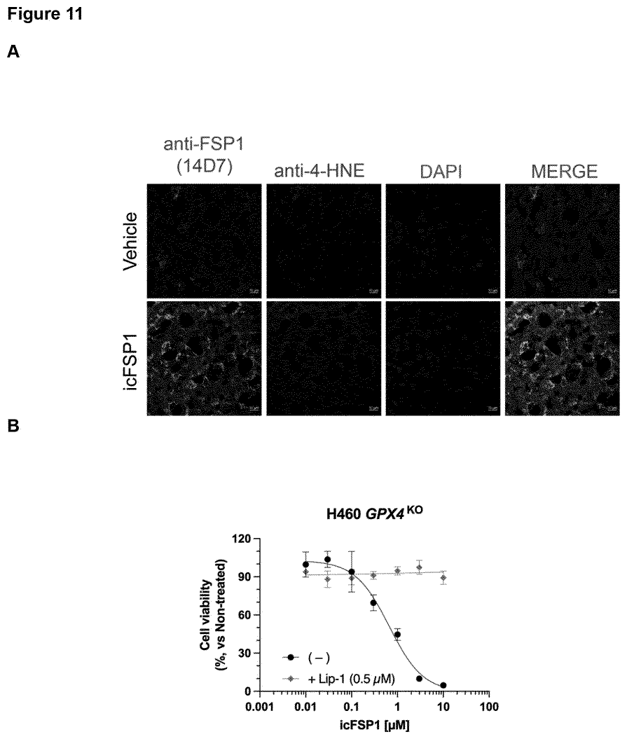

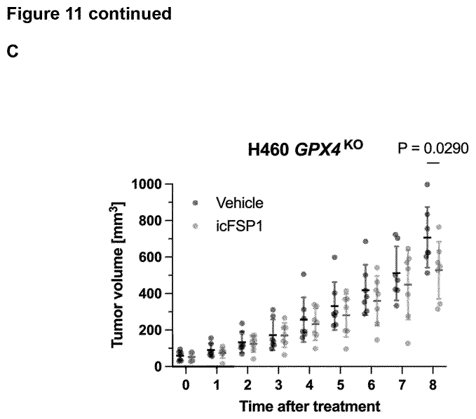

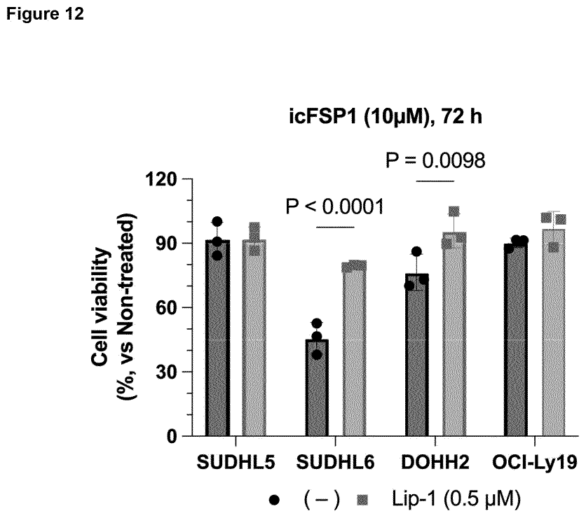

### DETAILED DESCRIPTION OF THE INVENTION

The solution of the present invention is described in the following, exemplified in the appended examples, illustrated in the Figures and reflected in the claims.□

### Definitions

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.□ The term “alkyl” refers to a monoradical of a saturated straight or branched hydrocarbon.

Preferably, the alkyl group comprises from 1 to 10 carbon atoms, i.e., 1, 2, 3, 4, 5, 6, 7, 8, 9, 10, or carbon atoms, more preferably 1 to 6 carbon atoms, such as 1 to 6 or 1 to 4 carbon atoms, more preferably 1 carbon atom Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, and the like.

The term “cycloalkyl” represents cyclic non-aromatic versions of “alkyl” with preferably 3 to 10 carbon atoms, i.e., 3, 4, 5, 6, 7, 8, 9, or 10 carbon atoms, more preferably 3 to 8 carbon atoms, even more preferably 3 to 6 carbon atoms. Exemplary cycloalkyl groups include cyclopropyl, cyclobutyl, cyclopentyl, cyclohexyl, cycloheptyl, cyclooctyl, cyclononyl, cylcodecyl, and adamantyl. Preferred examples of cycloalkyl include C₃-C₆-cycloalkyl, in particular cyclopropyl, cyclobutyl, cyclopentyl, cyclohexyl.

### Compounds

The invention is directed to a compound according to formula (I) or (II), preferably formula (I) for use in the treatment of cancer,

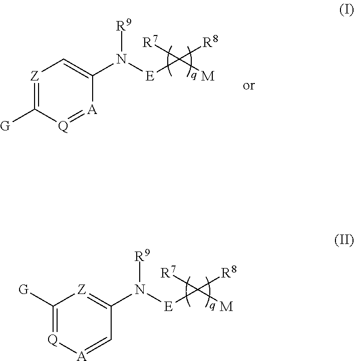

wherein G is selected from the group consisting of

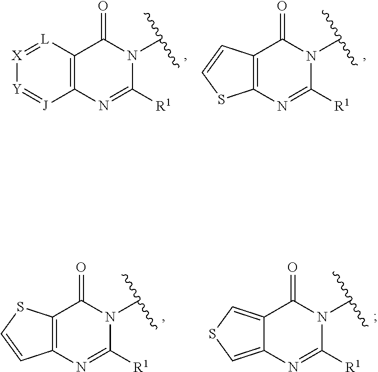

M is selected from the group consisting of

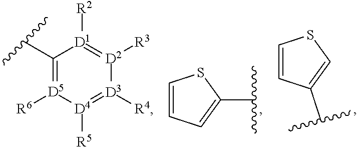

Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
L is selected from the group consisting of CH, N, C—F, C—Cl, and C—(C₁-C₆)alkyl, preferably CH;
X is selected from the group consisting of CH, N, C—F, C—Cl, and C—(C₁-C₆)alkyl, preferably CH;
Y is selected from the group consisting of CH, N, C—F, C—Cl, and C—(C₁-C₆)alkyl, preferably CH;
X and Y may be part of a ring according to formula (III):

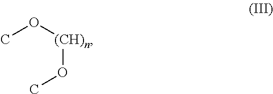

an example for a compound comprising a ring according to formula (III) is compound 47;
n is an integer from 1 to 3;
J is selected from the group consisting of CH, N, C—F, C—Cl, C—(C₁-C₆)alkyl, preferably CH;
D¹, D², D³, D4, D⁵, are independently selected from the group consisting of C and N; preferably C; wherein if any one of D¹, D², D³, D4, and/or D⁵ is N, then the corresponding R², R³, R⁴, R⁵ and/or R⁶ is absent. For example, if D¹ is N then R² is absent.
R¹ is selected from the group consisting of —(C₁-C₆)alkyl, —(C₃-C₆)cycloalkyl, —CH₂OH, —CH₂F, —CHF₂, and —CF₃, preferably —(C₁-C₆)alkyl;
R² is selected from the group consisting of H, —(C₁-C₆)alkyl, —CF₃, —C, and —F, preferably H;
R³ is selected from the group consisting of H, —(C₁-C₆)alkyl, —O(C₁-C₆)alkyl, —CF₃, —Cl, and —F, preferably —O(C₁-C₆)alkyl;
R⁴ is selected from the group consisting of H, —(C₁-C₆)alkyl, —O(C₁-C₆)alkyl, —CF₃, —C, and —F, preferably —O(C₁-C₆)alkyl;
R⁵ is selected from the group consisting of H, —(C₁-C₆)alkyl, —O(C₁-C₆)alkyl, —CF₃, —C, and —F, preferably —O(C₁-C₆)alkyl;
R⁶ is selected from the group consisting of H, —(C₁-C₆)alkyl, —CF₃, —C, and —F, preferably H;
two groups of R², R³, R⁴, R⁵ and R⁶ in vicinal position may be part of a ring selected from

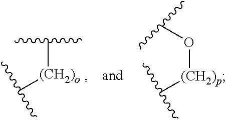

is an integer between 1 and 4;
p is an integer between 1 and 4;
q is 1 or 0;
R⁷ and R⁸ are independently selected from the group consisting of H, and —(C₁-C₆)alkyl;
R⁷ and R⁸ may form a 3 to 5 membered cycloalkyl;
R⁹ is selected from the group consisting of H, and —(C₁-C₆)alkyl or
a pharmaceutically acceptable salt thereof.

In a further embodiment Q, Z and/or A are C—OH₃.

In a further embodiment the compound according to formula (I) or (11) is not selected from the following compounds or a pharmaceutically acceptable salt thereof:

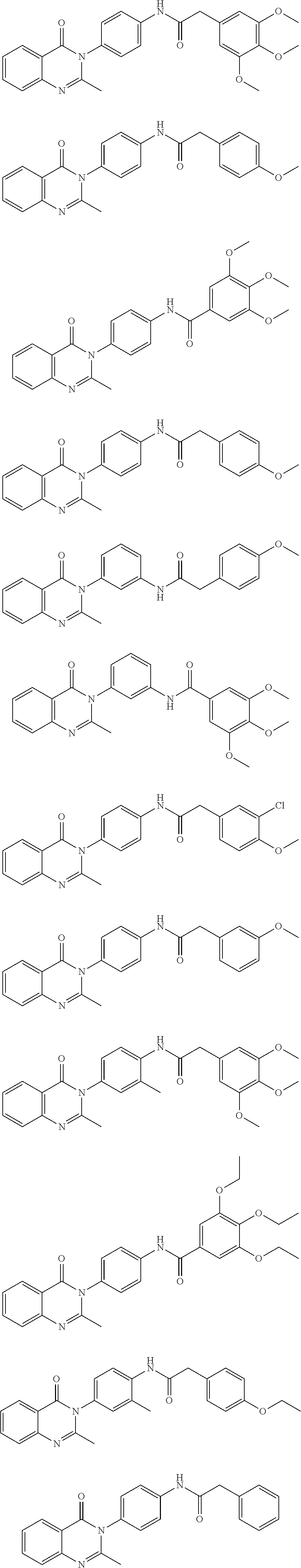

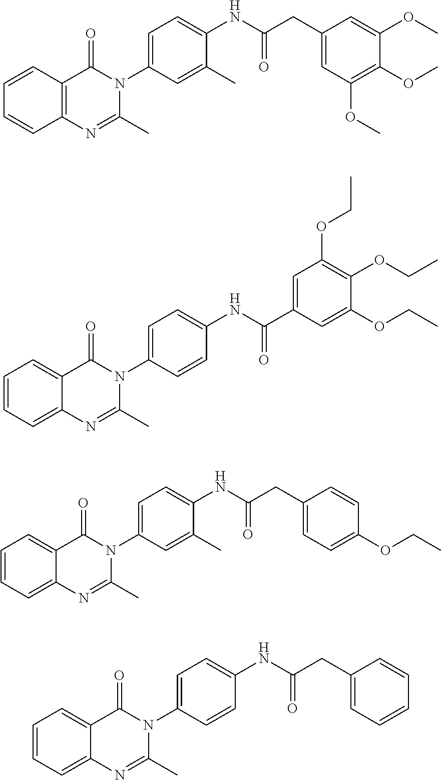 [SMILES: COc1cc(CC(=O)Nc2ccc(-n3c(C)nc4ccccc4c3=O)cc2C)cc(OC)c1OC ; CCOc1cc(C(=O)Nc2ccc(-n3c(C)nc4ccccc4c3=O)cc2)cc(OCC)c1OCC ; CCOc1ccc(CC(=O)Nc2ccc(-n3c(C)nc4ccccc4c3=O)cc2C)cc1 ; Cc1nc2ccccc2c(=O)n1-c1ccc(NC(=O)Cc2ccccc2)cc1]

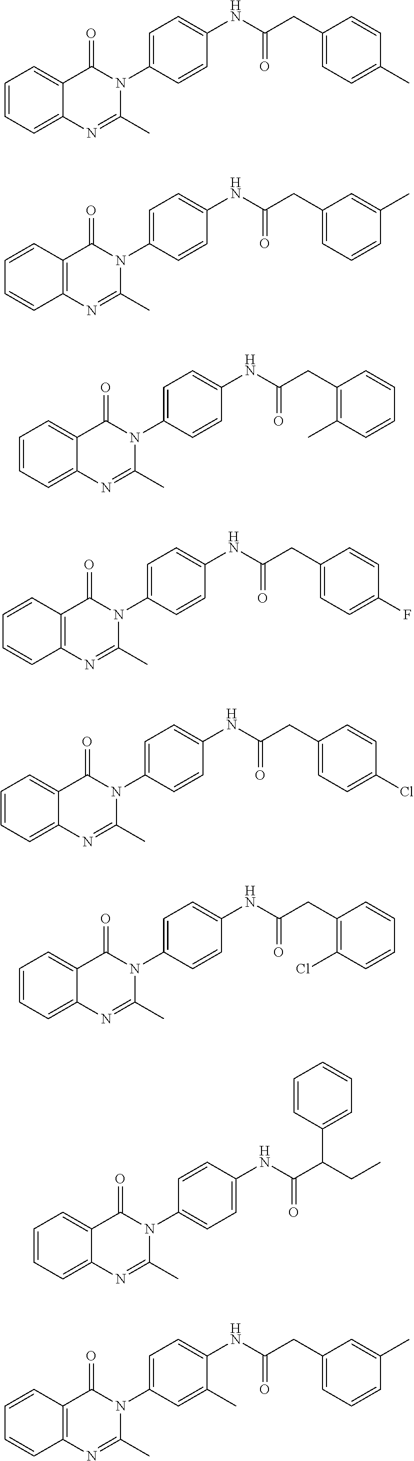

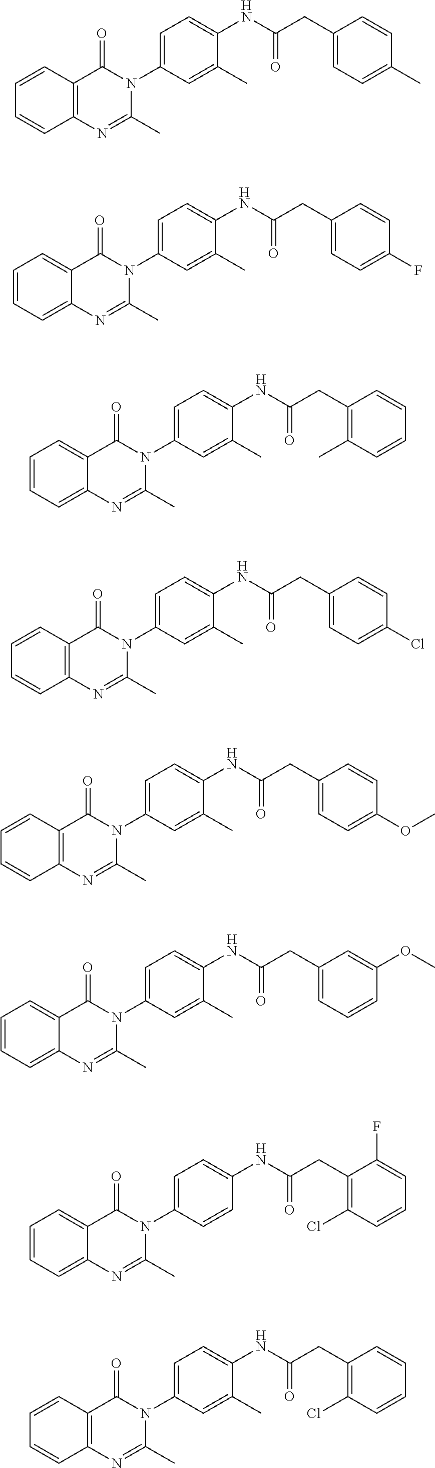

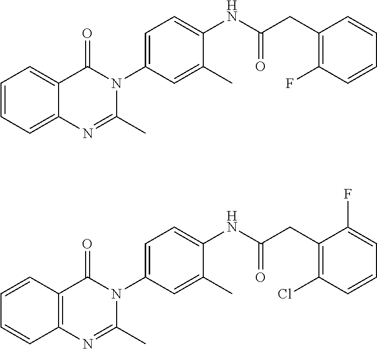 [SMILES: Cc1cc(-n2c(C)nc3ccccc3c2=O)ccc1NC(=O)Cc1ccccc1F ; Cc1cc(-n2c(C)nc3ccccc3c2=O)ccc1NC(=O)Cc1c(F)cccc1Cl]

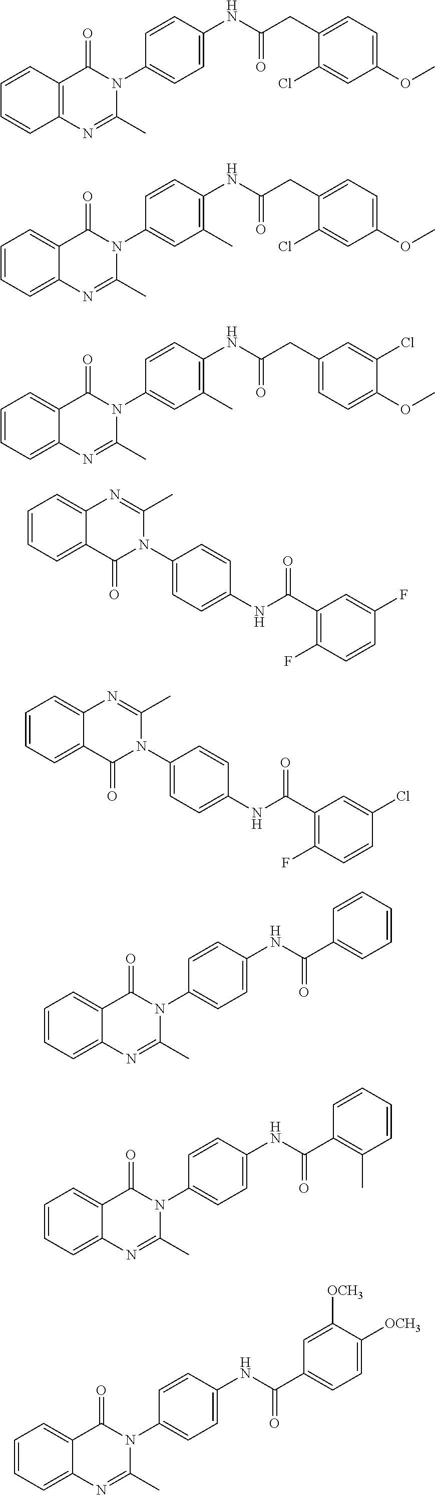

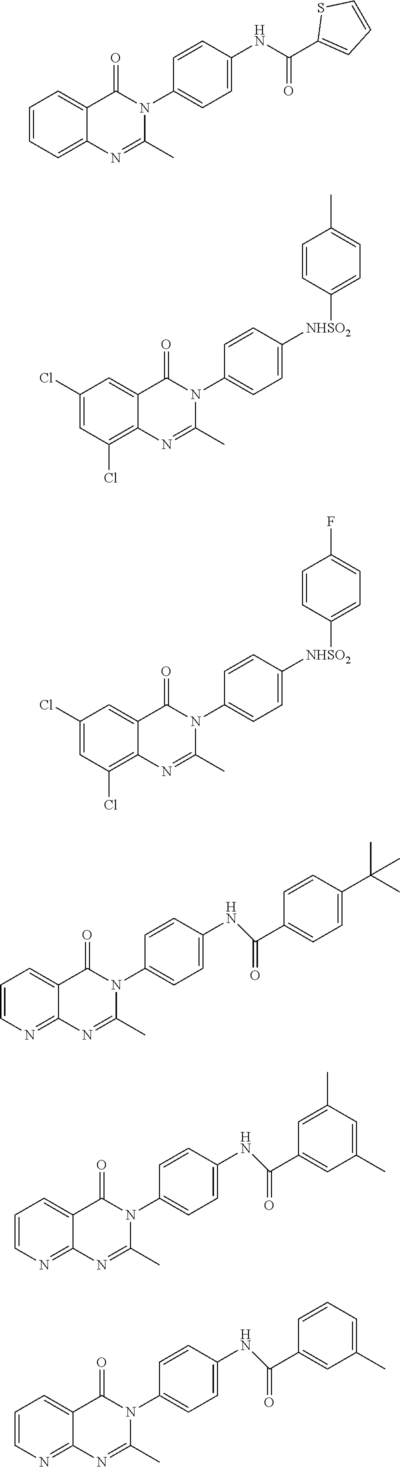

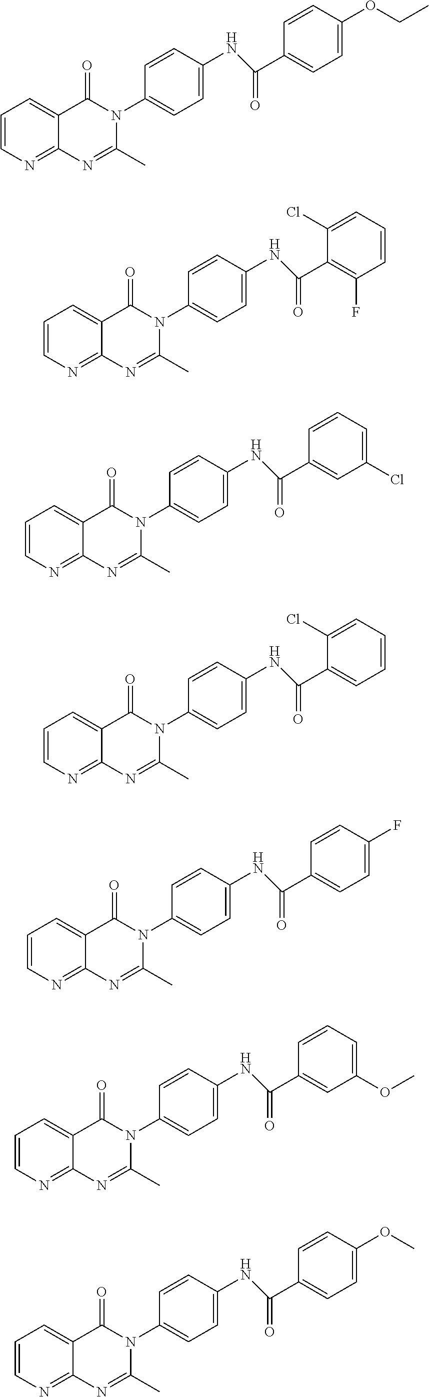

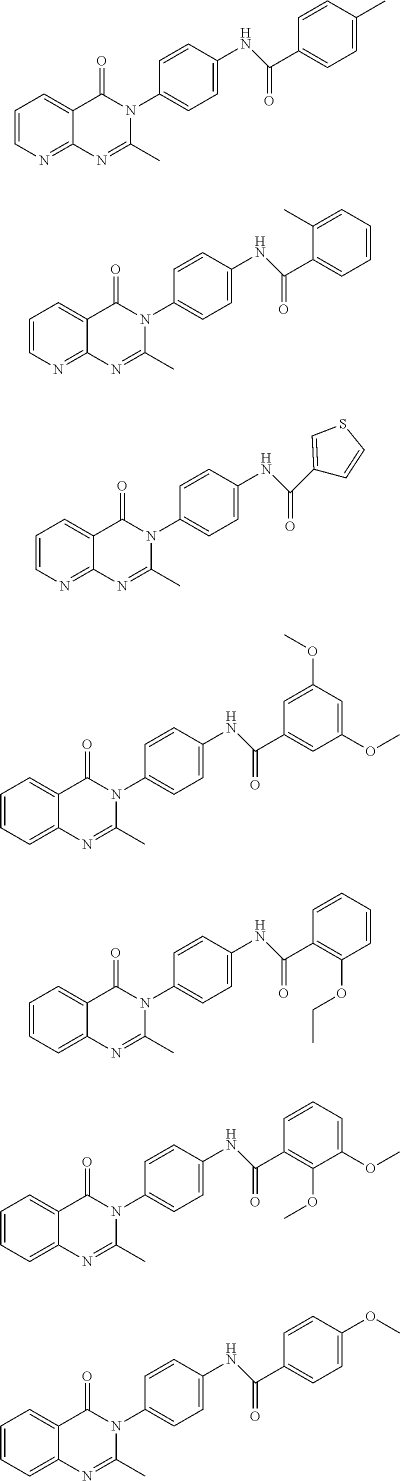

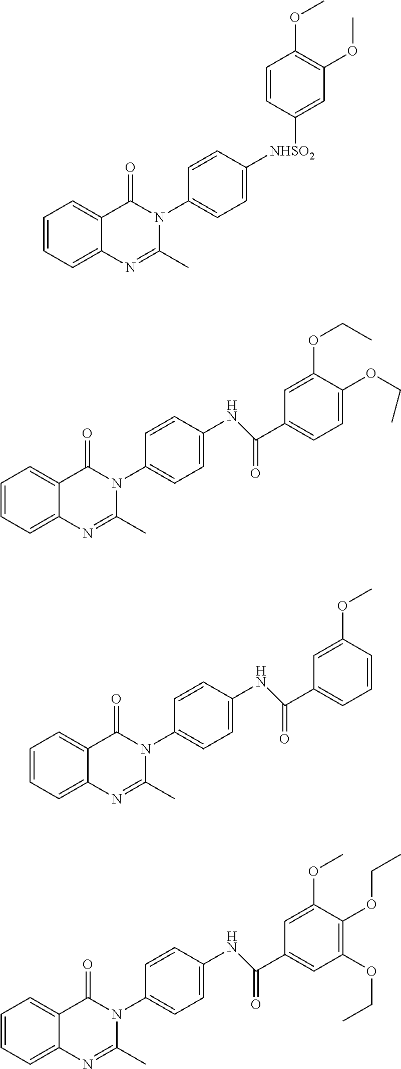

In a further embodiment, in formula (I) or (II),
G is

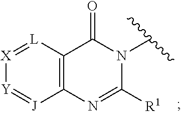

Z is CH;
A is CH;
Q is CH;
E is —CO—;
L is CH;
X is CH;
Y is preferably CH;
n is an integer from 1 to 3;
J is CH;
R¹ is —(C₁-C₆)alkyl; preferably methyl.
R² is H;
R³ is selected from the group consisting of H or —O(C₁-C₆)alkyl;
R⁴ is selected from the group consisting of H, or —O(C₁-C₆)alkyl;
R⁵ is selected from the group consisting of H, —Cl, or —O(C₁-C₆)alkyl;
R⁶ is selected from the group consisting of H or —Cl; preferably H;
o is an integer between 1 and 4;
p is an integer between 1 and 4;
q is 0 or 1;
R⁷ and R⁸ are independently selected from the group consisting of H, and —(C₁-C₆)alkyl;
R⁷ and R⁸ may form a 3 to 5 membered cycloalkyl;
R⁹ is selected from the group consisting of H, and —(C₁-C₆)alkyl.

TABLE 1
Specific Compounds of the present invention

| | |
|---|---|
| 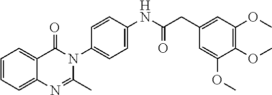 [SMILES: COc1cc(CC(=O)Nc2ccc(-n3c(C)nc4ccccc4c3=O)cc2)cc(OC)c1OC] | 1 |
| 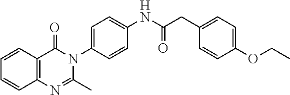 [SMILES: CCOc1ccc(CC(=O)Nc2ccc(-n3c(C)nc4ccccc4c3=O)cc2)cc1] | 2 |
| 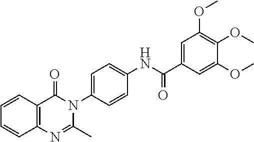 [SMILES: COc1cc(C(=O)Nc2ccc(-n3c(C)nc4ccccc4c3=O)cc2)cc(OC)c1OC] | 3 |
| 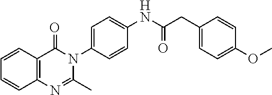 [SMILES: COc1ccc(CC(=O)Nc2ccc(-n3c(C)nc4ccccc4c3=O)cc2)cc1] | 4 |
| 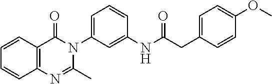 [SMILES: COc1ccc(CC(=O)Nc2cccc(-n3c(C)nc4ccccc4c3=O)c2)cc1] | 5 |
| 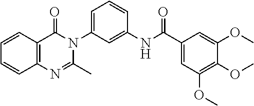 [SMILES: COc1cc(C(=O)Nc2cccc(-n3c(C)nc4ccccc4c3=O)c2)cc(OC)c1OC] | 6 |
| 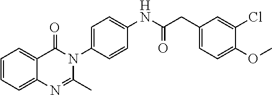 [SMILES: COc1ccc(CC(=O)Nc2ccc(-n3c(C)nc4ccccc4c3=O)cc2)cc1Cl] | 7 |
| 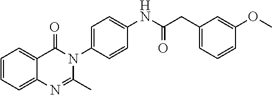 [SMILES: COc1cccc(CC(=O)Nc2ccc(-n3c(C)nc4ccccc4c3=O)cc2)c1] | 8 |
| 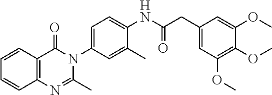 [SMILES: COc1cc(CC(=O)Nc2ccc(-n3c(C)nc4ccccc4c3=O)cc2C)cc(OC)c1OC] | 9 |
| 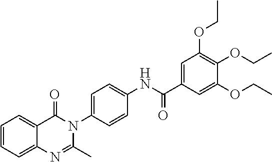 [SMILES: CCOc1cc(C(=O)Nc2ccc(-n3c(C)nc4ccccc4c3=O)cc2)cc(OCC)c1OCC] | 10 |
| 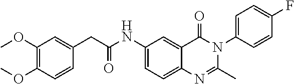 [SMILES: COc1ccc(CC(=O)Nc2ccc3nc(C)n(-c4ccc(F)cc4)c(=O)c3c2)cc1OC] | 11 |
| 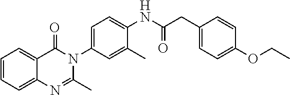 [SMILES: CCOc1ccc(CC(=O)Nc2ccc(-n3c(C)nc4ccccc4c3=O)cc2C)cc1] | 12 |
| 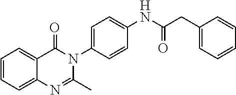 [SMILES: Cc1nc2ccccc2c(=O)n1-c1ccc(NC(=O)Cc2ccccc2)cc1] | 13 |
| 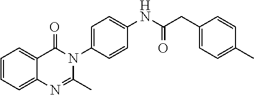 [SMILES: Cc1ccc(CC(=O)Nc2ccc(-n3c(C)nc4ccccc4c3=O)cc2)cc1] | 14 |
| 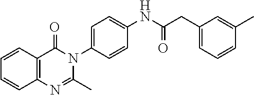 [SMILES: Cc1cccc(CC(=O)Nc2ccc(-n3c(C)nc4ccccc4c3=O)cc2)c1] | 15 |
| 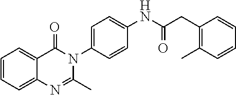 [SMILES: Cc1ccccc1CC(=O)Nc1ccc(-n2c(C)nc3ccccc3c2=O)cc1] | 16 |
| 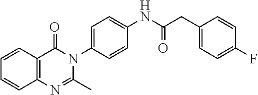 [SMILES: Cc1nc2ccccc2c(=O)n1-c1ccc(NC(=O)Cc2ccc(F)cc2)cc1] | 17 |
| 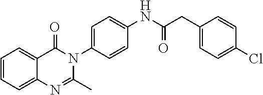 [SMILES: Cc1nc2ccccc2c(=O)n1-c1ccc(NC(=O)Cc2ccc(Cl)cc2)cc1] | 18 |
| 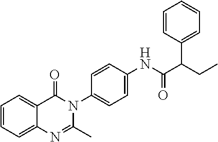 [SMILES: CCC(C(=O)Nc1ccc(-n2c(C)nc3ccccc3c2=O)cc1)c1ccccc1] | 19 |
| 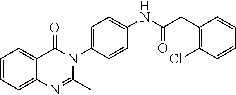 [SMILES: Cc1nc2ccccc2c(=O)n1-c1ccc(NC(=O)Cc2ccccc2Cl)cc1] | 20 |
| 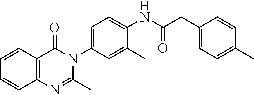 [SMILES: Cc1ccc(CC(=O)Nc2ccc(-n3c(C)nc4ccccc4c3=O)cc2C)cc1] | 21 |
| 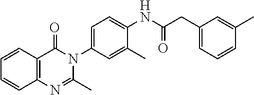 [SMILES: Cc1cccc(CC(=O)Nc2ccc(-n3c(C)nc4ccccc4c3=O)cc2C)c1] | 22 |
| 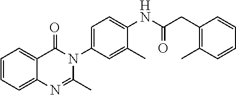 [SMILES: Cc1ccccc1CC(=O)Nc1ccc(-n2c(C)nc3ccccc3c2=O)cc1C] | 23 |
| 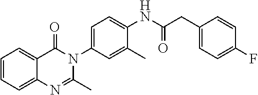 [SMILES: Cc1cc(-n2c(C)nc3ccccc3c2=O)ccc1NC(=O)Cc1ccc(F)cc1] | 24 |
| 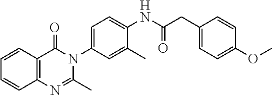 [SMILES: COc1ccc(CC(=O)Nc2ccc(-n3c(C)nc4ccccc4c3=O)cc2C)cc1] | 25 |
| 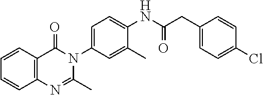 [SMILES: Cc1cc(-n2c(C)nc3ccccc3c2=O)ccc1NC(=O)Cc1ccc(Cl)cc1] | 26 |
| 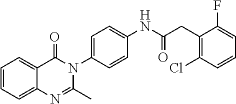 [SMILES: Cc1nc2ccccc2c(=O)n1-c1ccc(NC(=O)Cc2c(F)cccc2Cl)cc1] | 27 |
| 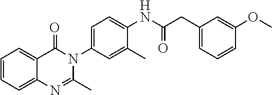 [SMILES: COc1cccc(CC(=O)Nc2ccc(-n3c(C)nc4ccccc4c3=O)cc2C)c1] | 28 |
| 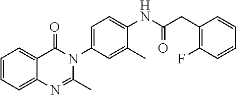 [SMILES: Cc1cc(-n2c(C)nc3ccccc3c2=O)ccc1NC(=O)Cc1ccccc1F] | 29 |
| 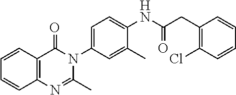 [SMILES: Cc1cc(-n2c(C)nc3ccccc3c2=O)ccc1NC(=O)Cc1ccccc1Cl] | 30 |
| 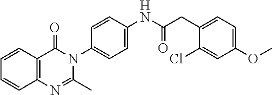 [SMILES: COc1ccc(CC(=O)Nc2ccc(-n3c(C)nc4ccccc4c3=O)cc2)c(Cl)c1] | 31 |
| 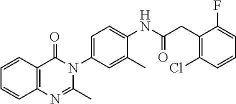 [SMILES: Cc1cc(-n2c(C)nc3ccccc3c2=O)ccc1NC(=O)Cc1c(F)cccc1Cl] | 32 |
| 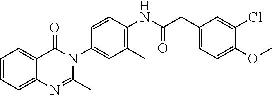 [SMILES: COc1ccc(CC(=O)Nc2ccc(-n3c(C)nc4ccccc4c3=O)cc2C)cc1Cl] | 33 |
| 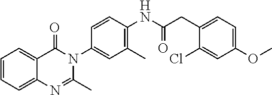 [SMILES: COc1ccc(CC(=O)Nc2ccc(-n3c(C)nc4ccccc4c3=O)cc2C)c(Cl)c1] | 34 |
| 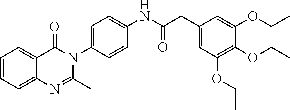 [SMILES: CCOc1cc(CC(=O)Nc2ccc(-n3c(C)nc4ccccc4c3=O)cc2)cc(OCC)c1OCC] | 35 |
| 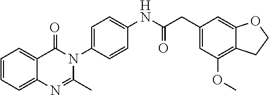 [SMILES: COc1cc(CC(=O)Nc2ccc(-n3c(C)nc4ccccc4c3=O)cc2)cc2c1CCO2] | 36 |
| 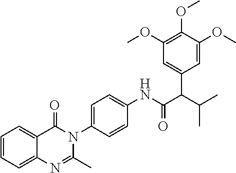 [SMILES: COc1cc(C(C(=O)Nc2ccc(-n3c(C)nc4ccccc4c3=O)cc2)C(C)C)cc(OC)c1OC] | 37 |
| 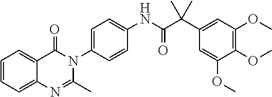 [SMILES: COc1cc(C(C)(C)C(=O)Nc2ccc(-n3c(C)nc4ccccc4c3=O)cc2)cc(OC)c1OC] | 38 |
| 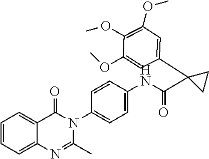 [SMILES: COc1cc(C2(C(=O)Nc3ccc(-n4c(C)nc5ccccc5c4=O)cc3)CC2)cc(OC)c1OC] | 39 |
| 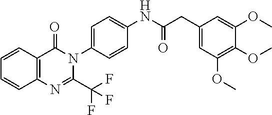 [SMILES: COc1cc(CC(=O)Nc2ccc(-n3c(C(F)(F)F)nc4ccccc4c3=O)cc2)cc(OC)c1OC] | 40 |
| 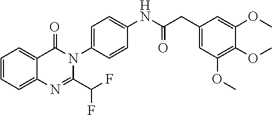 [SMILES: COc1cc(CC(=O)Nc2ccc(-n3c(C(F)F)nc4ccccc4c3=O)cc2)cc(OC)c1OC] | 41 |
| 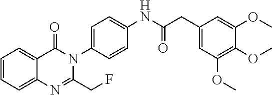 [SMILES: COc1cc(CC(=O)Nc2ccc(-n3c(CF)nc4ccccc4c3=O)cc2)cc(OC)c1OC] | 42 |
| 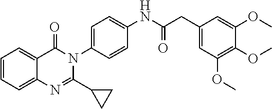 [SMILES: COc1cc(CC(=O)Nc2ccc(-n3c(C4CC4)nc4ccccc4c3=O)cc2)cc(OC)c1OC] | 43 |
| 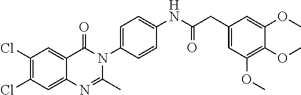 [SMILES: COc1cc(CC(=O)Nc2ccc(-n3c(C)nc4cc(Cl)c(Cl)cc4c3=O)cc2)cc(OC)c1OC] | 44 |
| 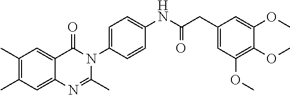 | 45 |
| 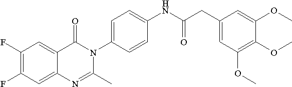 [SMILES: COc1cc(CC(=O)Nc2ccc(-n3c(C)nc4cc(F)c(F)cc4c3=O)cc2)cc(OC)c1OC] | 46 |
| 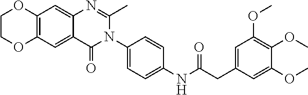 [SMILES: COc1cc(CC(=O)Nc2ccc(-n3c(C)nc4cc5c(cc4c3=O)OCCO5)cc2)cc(OC)c1OC] | 47 |
| 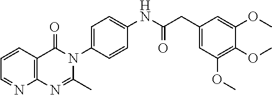 [SMILES: COc1cc(CC(=O)Nc2ccc(-n3c(C)nc4ncccc4c3=O)cc2)cc(OC)c1OC] | 48 |
| 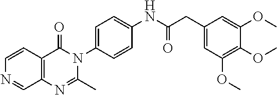 [SMILES: COc1cc(CC(=O)Nc2ccc(-n3c(C)nc4cnccc4c3=O)cc2)cc(OC)c1OC] | 49 |
| 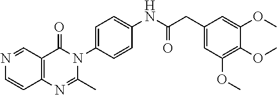 [SMILES: COc1cc(CC(=O)Nc2ccc(-n3c(C)nc4ccncc4c3=O)cc2)cc(OC)c1OC] | 50 |
| 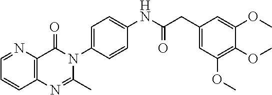 [SMILES: COc1cc(CC(=O)Nc2ccc(-n3c(C)nc4cccnc4c3=O)cc2)cc(OC)c1OC] | 51 |
| 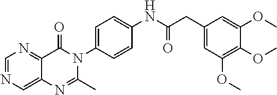 [SMILES: COc1cc(CC(=O)Nc2ccc(-n3c(C)nc4cncnc4c3=O)cc2)cc(OC)c1OC] | 52 |
| 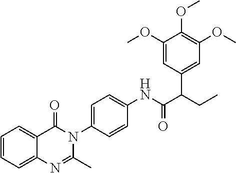 [SMILES: CCC(C(=O)Nc1ccc(-n2c(C)nc3ccccc3c2=O)cc1)c1cc(OC)c(OC)c(OC)c1] | 53 |
| 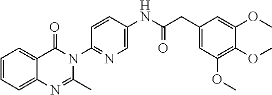 [SMILES: COc1cc(CC(=O)Nc2ccc(-n3c(C)nc4ccccc4c3=O)nc2)cc(OC)c1OC] | 54 |
| 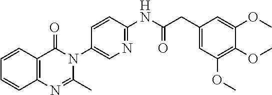 [SMILES: COc1cc(CC(=O)Nc2ccc(-n3c(C)nc4ccccc4c3=O)cn2)cc(OC)c1OC] | 55 |
| 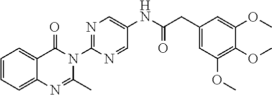 [SMILES: COc1cc(CC(=O)Nc2cnc(-n3c(C)nc4ccccc4c3=O)nc2)cc(OC)c1OC] | 56 |
| 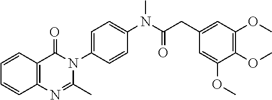 [SMILES: COc1cc(CC(=O)N(C)c2ccc(-n3c(C)nc4ccccc4c3=O)cc2)cc(OC)c1OC] | 57 |
| 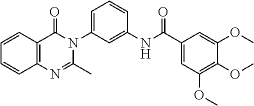 [SMILES: COc1cc(C(=O)Nc2cccc(-n3c(C)nc4ccccc4c3=O)c2)cc(OC)c1OC] | 58 |
| 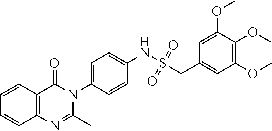 [SMILES: COc1cc(CS(=O)(=O)Nc2ccc(-n3c(C)nc4ccccc4c3=O)cc2)cc(OC)c1OC] | 59 |
| 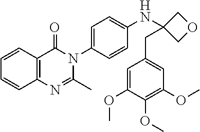 [SMILES: COc1cc(CC2(Nc3ccc(-n4c(C)nc5ccccc5c4=O)cc3)COC2)cc(OC)c1OC] | 60 |
| 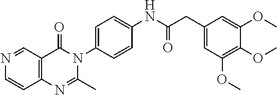 [SMILES: COc1cc(CC(=O)Nc2ccc(-n3c(C)nc4ccncc4c3=O)cc2)cc(OC)c1OC] | 61 |
| 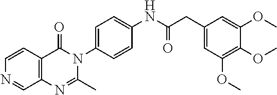 [SMILES: COc1cc(CC(=O)Nc2ccc(-n3c(C)nc4cnccc4c3=O)cc2)cc(OC)c1OC] | 62 |
| 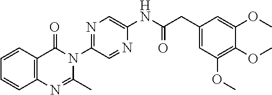 [SMILES: COc1cc(CC(=O)Nc2cnc(-n3c(C)nc4ccccc4c3=O)cn2)cc(OC)c1OC] | 63 |
| 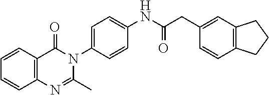 [SMILES: Cc1nc2ccccc2c(=O)n1-c1ccc(NC(=O)Cc2ccc3c(c2)CCC3)cc1] | 64 |
| 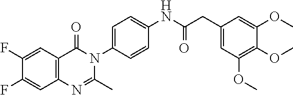 [SMILES: COc1cc(CC(=O)Nc2ccc(-n3c(C)nc4cc(F)c(F)cc4c3=O)cc2)cc(OC)c1OC] | 65 |
| 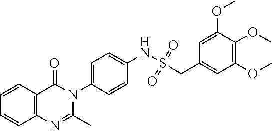 [SMILES: COc1cc(CS(=O)(=O)Nc2ccc(-n3c(C)nc4ccccc4c3=O)cc2)cc(OC)c1OC] | 66 |
| 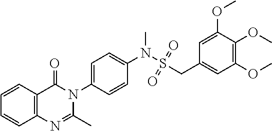 [SMILES: COc1cc(CS(=O)(=O)N(C)c2ccc(-n3c(C)nc4ccccc4c3=O)cc2)cc(OC)c1OC] | 67 |
| 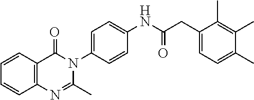 [SMILES: Cc1ccc(CC(=O)Nc2ccc(-n3c(C)nc4ccccc4c3=O)cc2)c(C)c1C] | 68 |
| 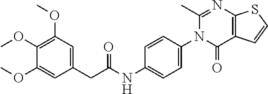 [SMILES: COc1cc(CC(=O)Nc2ccc(-n3c(C)nc4sccc4c3=O)cc2)cc(OC)c1OC] | 69 |
| 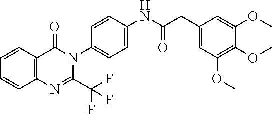 [SMILES: COc1cc(CC(=O)Nc2ccc(-n3c(C(F)(F)F)nc4ccccc4c3=O)cc2)cc(OC)c1OC] | 70 |
| 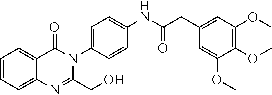 [SMILES: COc1cc(CC(=O)Nc2ccc(-n3c(CO)nc4ccccc4c3=O)cc2)cc(OC)c1OC] | 71 |
| 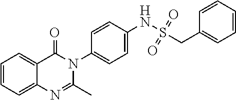 [SMILES: Cc1nc2ccccc2c(=O)n1-c1ccc(NS(=O)(=O)Cc2ccccc2)cc1] | 72 |
| 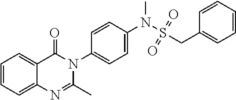 [SMILES: Cc1nc2ccccc2c(=O)n1-c1ccc(N(C)S(=O)(=O)Cc2ccccc2)cc1] | 73 |
| 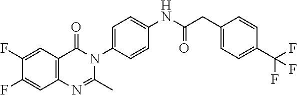 [SMILES: Cc1nc2cc(F)c(F)cc2c(=O)n1-c1ccc(NC(=O)Cc2ccc(C(F)(F)F)cc2)cc1] | 74 |
| 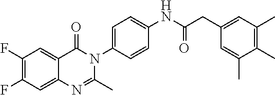 [SMILES: Cc1cc(CC(=O)Nc2ccc(-n3c(C)nc4cc(F)c(F)cc4c3=O)cc2)cc(C)c1C] | 75 |
| 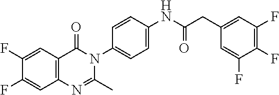 [SMILES: Cc1nc2cc(F)c(F)cc2c(=O)n1-c1ccc(NC(=O)Cc2cc(F)c(F)c(F)c2)cc1] | 76 |
| 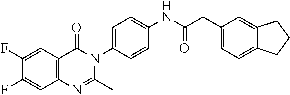 [SMILES: Cc1nc2cc(F)c(F)cc2c(=O)n1-c1ccc(NC(=O)Cc2ccc3c(c2)CCC3)cc1] | 78 |
| 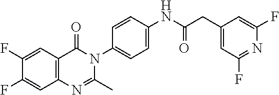 [SMILES: Cc1nc2cc(F)c(F)cc2c(=O)n1-c1ccc(NC(=O)Cc2cc(F)nc(F)c2)cc1] | 79 |
| 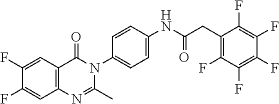 [SMILES: Cc1nc2cc(F)c(F)cc2c(=O)n1-c1ccc(NC(=O)Cc2c(F)c(F)c(F)c(F)c2F)cc1] | 80 |
| 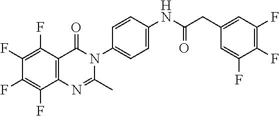 [SMILES: Cc1nc2c(F)c(F)c(F)c(F)c2c(=O)n1-c1ccc(NC(=O)Cc2cc(F)c(F)c(F)c2)cc1] | 81 |
| 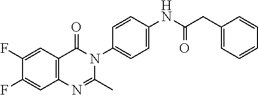 [SMILES: Cc1nc2cc(F)c(F)cc2c(=O)n1-c1ccc(NC(=O)Cc2ccccc2)cc1] | 82 |
| 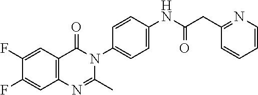 [SMILES: Cc1nc2cc(F)c(F)cc2c(=O)n1-c1ccc(NC(=O)Cc2ccccn2)cc1] | 83 |
| 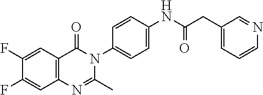 [SMILES: Cc1nc2cc(F)c(F)cc2c(=O)n1-c1ccc(NC(=O)Cc2cccnc2)cc1] | 84 |
| 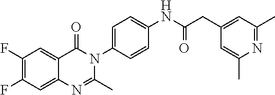 [SMILES: Cc1cc(CC(=O)Nc2ccc(-n3c(C)nc4cc(F)c(F)cc4c3=O)cc2)cc(C)n1] | 85 |
| 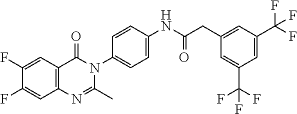 [SMILES: Cc1nc2cc(F)c(F)cc2c(=O)n1-c1ccc(NC(=O)Cc2cc(C(F)(F)F)cc(C(F)(F)F)c2)cc1] | 86 |

In one embodiment the compound according to formula (I) and (II) is selected from the group consisting of

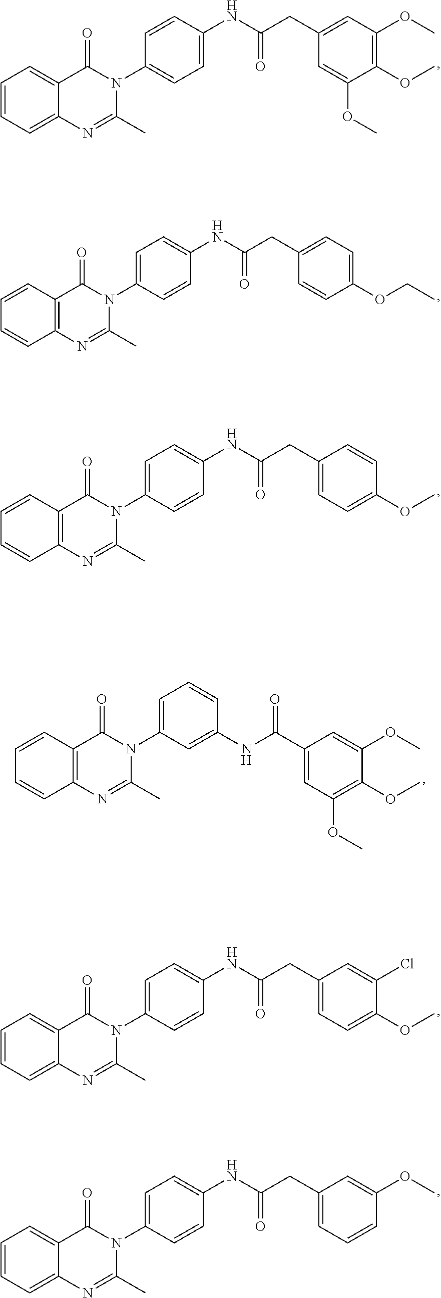 [SMILES: COc1cc(CC(=O)Nc2ccc(-n3c(C)nc4ccccc4c3=O)cc2)cc(OC)c1OC ; CCOc1ccc(CC(=O)Nc2ccc(-n3c(C)nc4ccccc4c3=O)cc2)cc1 ; COc1ccc(CC(=O)Nc2ccc(-n3c(C)nc4ccccc4c3=O)cc2)cc1 ; COc1cc(C(=O)Nc2cccc(-n3c(C)nc4ccccc4c3=O)c2)cc(OC)c1OC ; COc1ccc(CC(=O)Nc2ccc(-n3c(C)nc4ccccc4c3=O)cc2)cc1Cl ; COc1cccc(CC(=O)Nc2ccc(-n3c(C)nc4ccccc4c3=O)cc2)c1]

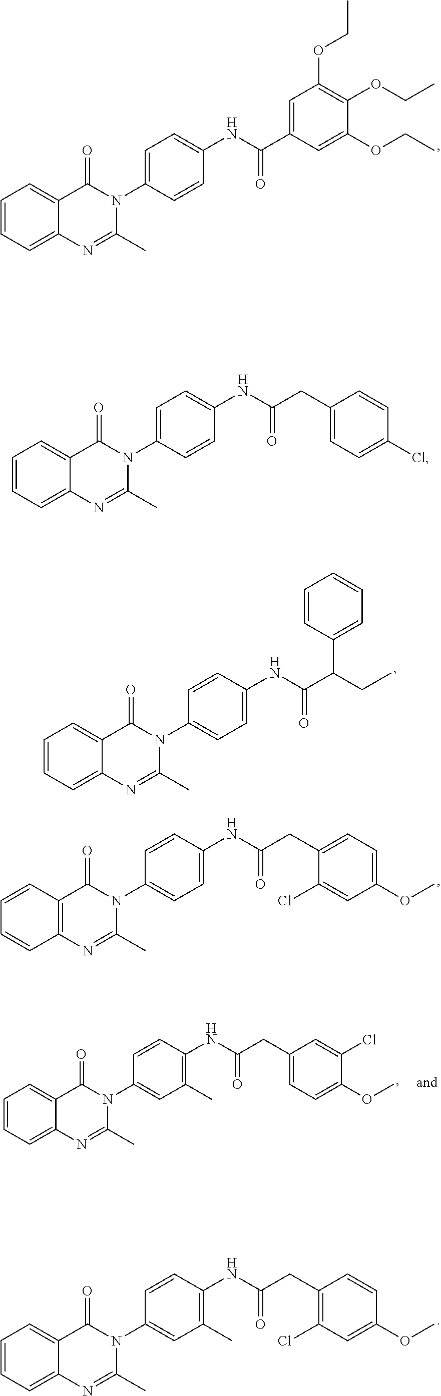

In a further embodiment, the compound according to formula (I) and (II) is selected from the group consisting of

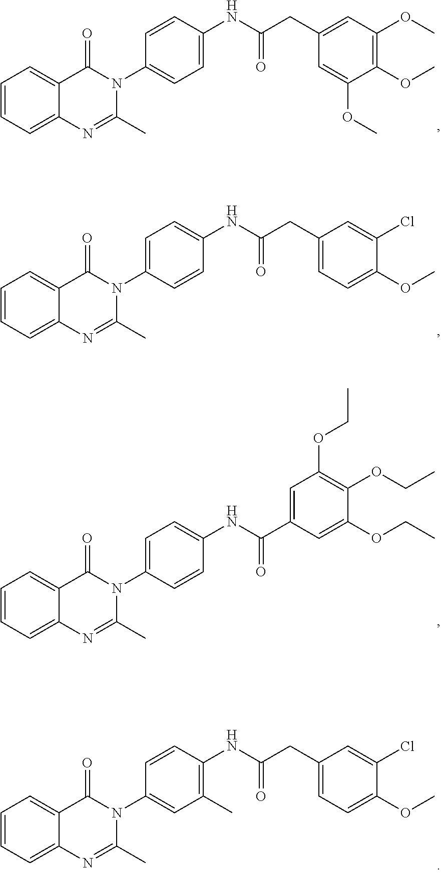 [SMILES: COc1cc(CC(=O)Nc2ccc(-n3c(C)nc4ccccc4c3=O)cc2)cc(OC)c1OC ; COc1ccc(CC(=O)Nc2ccc(-n3c(C)nc4ccccc4c3=O)cc2)cc1Cl ; CCOc1cc(C(=O)Nc2ccc(-n3c(C)nc4ccccc4c3=O)cc2)cc(OCC)c1OCC ; COc1ccc(CC(=O)Nc2ccc(-n3c(C)nc4ccccc4c3=O)cc2C)cc1Cl]

### Synthesis of Compounds

The synthesis comprises in general one or more of the following steps:

Step 1: In a first step compound (IV) or (VII) is acylated.

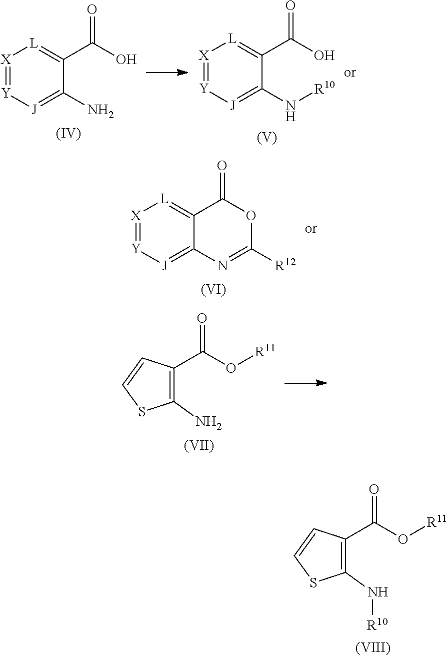

wherein
L is selected from the group consisting of CH, N, C—F, C—Cl, and C—(C₁-C₆)alkyl, preferably CH;
X is selected from the group consisting of CH, N, C—F, C—Cl, and C—(C₁-C₆)alkyl, preferably CH.
Y is selected from the group consisting of CH, N, C—F, C—Cl, and C—(C₁-C₆)alkyl, preferably CH.
X and Y may be part of ring according to formula (III):

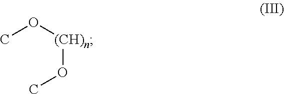

n is an integer from 1 to 3.
J is selected from the group consisting of CH, N, C—F, C—Cl, C—(C₁-C₆)alkyl, preferably CH.
R¹⁰ is —CO(C₁-C₆)alkyl, —CO(C₃-C₆)cycloalkyl, —COCH₂F, —COCHF₂, and —COCF₃, preferably —CO(C₁-C₆)alkyl.
R¹¹ is H, or (C₁-C₆)alkyl, preferably (C₁-C₆)alkyl.
R¹² is —(C₁-C₆)alkyl, —(C₃-C₆)cycloalkyl, —CH₂F, —CHF₂, and —CF₃, preferably —(C₁-C₆)alkyl.

Conditions for acylating an amine are well known to the person skilled in the art. For example, the acylation may be carried out using reagents based on a corresponding acetylhalogenide or anhydride. The acylation may be carried out in the presence of bases, such as Et₃N, Hunig base, or other suitable bases. Further acylation conditions may be found for example in Peter G. M. Wuts, Greene's Protective Groups in Organic Chemistry, Fifth Edition, Wiley 2014.

If R₁₁ is (C₁-C₆)alkyl a further hydrolysis step may be carried out, resulting in a compound wherein R₁₁ is H. The hydrolysis may be carried out using inorganic bases such as NaOH, KOH, LiOH in the presence of protic solvents, such as water and/or alcohols for example such as methanol, and ethanol. See for further conditions Peter G. M. Wuts, Greene's Protective Groups in Organic Chemistry, Fifth Edition, Wiley 2014.

### Step 2:

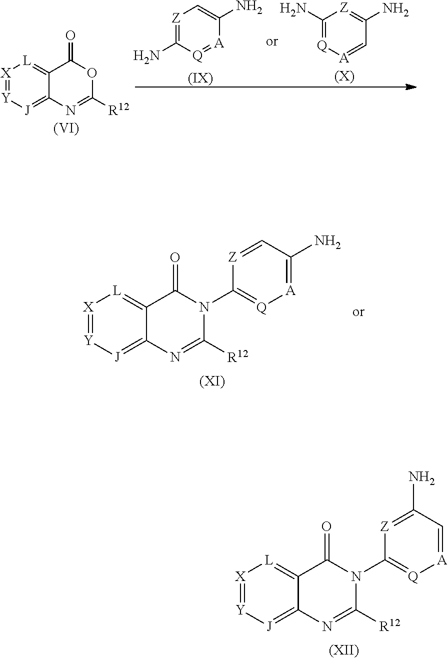

Z is selected from the group consisting of CH, and N, preferably CH;
A is selected from the group consisting of CH, and N, preferably CH;
Q is selected from the group consisting of CH, and N, preferably CH;
L is selected from the group consisting of CH, N, C—F, C—Cl, and C—(C₁-C₆)alkyl, preferably CH;
X is selected from the group consisting of CH, N, C—F, C—Cl, and C—(C₁-C₆)alkyl, preferably CH;
Y is selected from the group consisting of CH, N, C—F, C—Cl, and C—(C₁-C₆)alkyl, preferably CH;
X and Y may be part of ring according to formula (III):

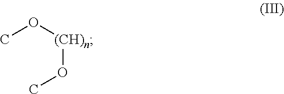

n is an integer from 1 to 3;
J is selected from the group consisting of CH, N, C—F, C—Cl, C—(C₁-C₆)alkyl, preferably CH;
R¹² is —(C₁-C₆)alkyl, —(C₃-C₆)cycloalkyl, —CH₂F, —CHF₂, and —CF₃, preferably —(C₁-C₆)alkyl.

Preferably, the reaction is carried out by heating the starting material compound (VI) with compound (IX) or (X) in pyridine at reflux until the starting material is completely converted, preferably for 4 to 10 h, more preferably 5 to 7 h, most preferably 6 h.

### Step 3:

In step 3 compound (V) is converted with compound (XIII) or compound (XIV) in order to obtain compound (Ia) or (IIa). In an analogous manner compound (VIII) wherein R¹¹ is H may be converted to the corresponding products.

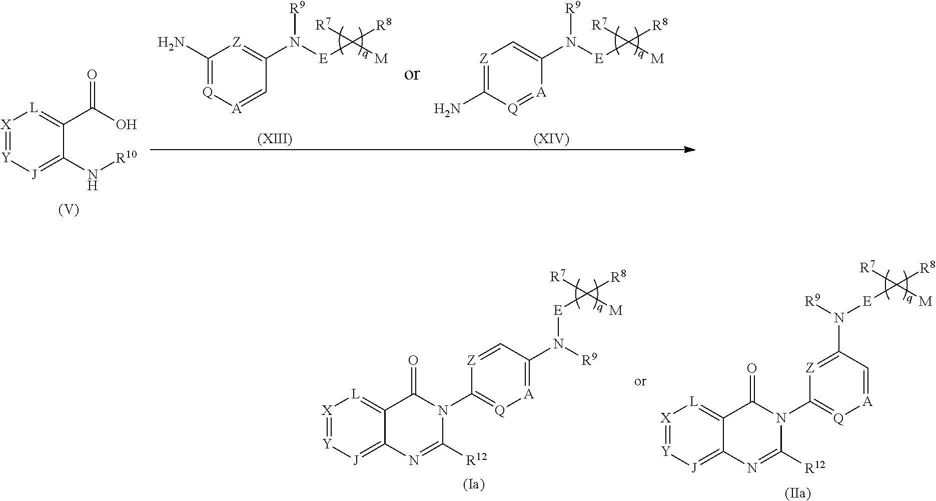

wherein
M is selected from the group consisting of

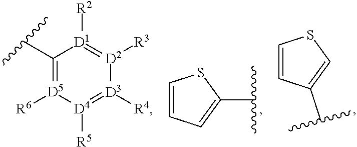

preferably

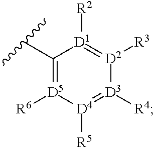

Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
L is selected from the group consisting of CH, N, C—F, C—Cl, and C—(C₁-C₆)alkyl, preferably CH; X is selected from the group consisting of CH, N, C—F, C—Cl, and C—(C₁-C₆)alkyl, preferably CH;
Y is selected from the group consisting of CH, N, C—F, C—Cl, and C—(C₁-C₆)alkyl, preferably CH;
X and Y may be part of ring according to formula (III):

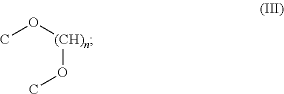

n is an integer from 1 to 3;
J is selected from the group consisting of CH, N, C—F, C—Cl, C—(C₁-C₆)alkyl, preferably CH;
D¹, D², D³, D⁴, D⁵, are independently selected from the group consisting of C and N; preferably C;
R¹ is selected from the group consisting of —(C₁-C₆)alkyl, —(C₃-C₆)cycloalkyl, —CH₂OH, —CH₂F, —CHF₂, and —CF₃, preferably —(C₁-C₆)alkyl;
R² is selected from the group consisting of H, —(C₁-C₆)alkyl, —CF₃, —Cl, and —F, preferably H;
R³ is selected from the group consisting of H, —(C₁-C₆)alkyl, —O(C₁-C₆)alkyl, —CF₃, —Cl, and —F, preferably —O(C₁-C₆)alkyl;
R⁴ is selected from the group consisting of H, —(C₁-C₆)alkyl, —O(C₁-C₆)alkyl, —CF₃, —Cl, and —F, preferably —O(C₁-C₆)alkyl;
R⁵ is selected from the group consisting of H, —(C₁-C₆)alkyl, —O(C₁-C₆)alkyl, —CF₃, —Cl, and —F, preferably —O(C₁-C₆)alkyl;
R⁶ is selected from the group consisting of H, —(C₁-C₆)alkyl, —CF₃, —C, and —F, preferably H;
two groups of R², R³, R⁴, R⁵ and R⁶ in vicinal position may be part of a ring selected from

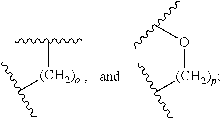

is an integer between 1 and 4;
p is an integer between 1 and 4;
q is 1 or 0;
R⁷ and R⁸ are independently selected from the group consisting of H, and —(C₁-C₆)alkyl;
R⁷ and R⁸ may form a 3 to 5 membered cycloalkyl ring;
R⁹ is selected from the group consisting of H, and —(C₁-C₆)alkyl;
R¹⁰ is —CO(C₁-C₆)alkyl, —CO(C₃-C₆)cycloalkyl, —COCH₂F, —COCHF₂, and —COCF₃, preferably —CO(C₁-C₆)alkyl;

The conversion in step 3 may be carried out by application of amid bond formation conditions known to the person skilled in the art. For example amid bond forming reagents such as EDCl (1-Ethyl-3-(3-dimethylaminopropyl)carbodiimid), in the presence of organic catalysts such as HOBt or DMAP (Dimethylaminopyridine) may be applied. Usually, the amid bond formation is carried out in the presence of a weak organic base such as NEt₃, or Et₂NiPr. See for further conditions Peter G. M. Wuts, Greene's Protective Groups in Organic Chemistry, Fifth Edition, Wiley 2014.

### Step 4:

Compound (XIII) and compound (XIV) may be synthesized by acylation of compound (IX) or (X) with compound (XV). Analogously, compound (XI) or (XII) may be acylated with compound (XV).

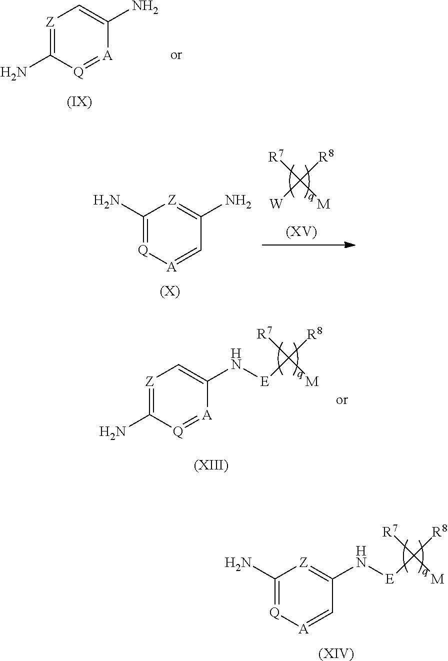

wherein
M is selected from the group consisting of

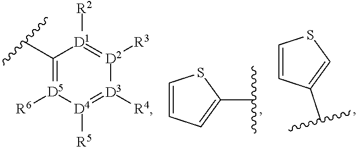

preferably

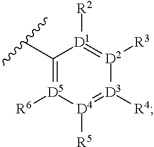

Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
D¹, D², D³, D⁴, D⁵, are independently selected from the group consisting of C and N; preferably C;
R¹ is selected from the group consisting of —(C₁-C₆)alkyl, —(C₃-C₆)cycloalkyl, —CH₂OH, —CH₂F, —CHF₂, and —CF₃, preferably —(C₁-C₆)alkyl;
R² is selected from the group consisting of H, —(C₁-C₆)alkyl, —CF₃, —Cl, and —F, preferably H;
R³ is selected from the group consisting of H, —(C₁-C₆)alkyl, —O(C₁-C₆)alkyl, —CF₃, —Cl, and —F, preferably —O(C₁-C₆)alkyl;
R⁴ is selected from the group consisting of H, —(C₁-C₆)alkyl, —O(C₁-C₆)alkyl, —CF₃, —Cl, and —F, preferably —O(C₁-C₆)alkyl;
R⁵ is selected from the group consisting of H, —(C₁-C₆)alkyl, —O(C₁-C₆)alkyl, —CF₃, —Cl, and —F, preferably —O(C₁-C₆)alkyl;
R⁶ is selected from the group consisting of H, —(C₁-C₆)alkyl, —CF₃, —Cl, and —F, preferably H; two groups of R², R³, R⁴, R⁵ and R⁶ in vicinal position may be part of a ring selected from

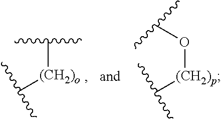

is an integer between 1 and 4;
p is an integer between 1 and 4;
q is 1 or 0;
R⁷ and R⁸ are independently selected from the group consisting of H, and —(C₁-C₆)alkyl;
R⁷ and R⁸ may form a 3 to 5 membered cycloalkyl;
R⁹ is selected from the group consisting of H, and —(C₁-C₆)alkyl;
W is —COOH, or —SO₂Cl;

The acylation may be carried out applying acylation conditions known in the art such as disclosed in Peter G. M. Wuts, Greene's Protective Groups in Organic Chemistry, Fifth Edition, Wiley 2014. In particular, the acylation may be carried out in the presence of HATU, and a weak organic base like NEt₃ and Et₂NiPr in a suitable solvent such as DMF.

### Step 5:

Compound (XVI) and compound (XVII) may be synthesized by acylation of compound (XI) or (XII) with compound (XV)

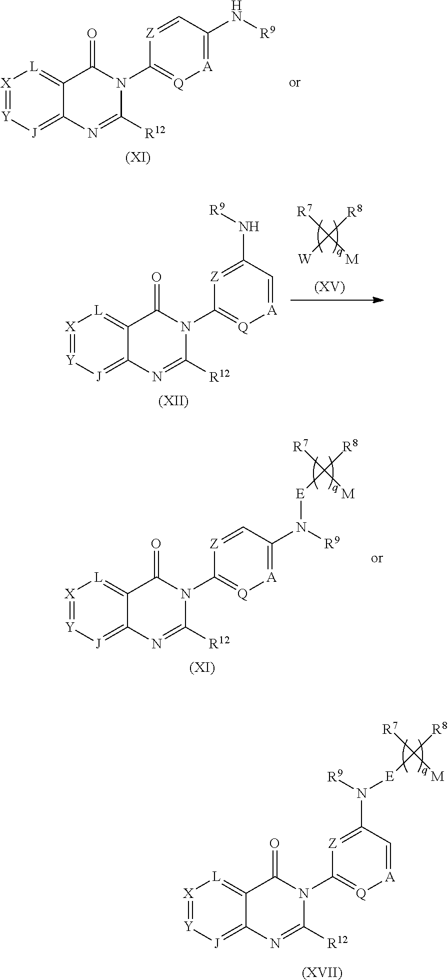

wherein
M is selected from the group consisting of

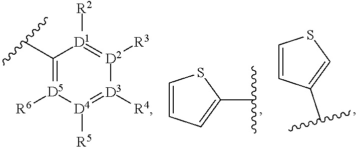

preferably

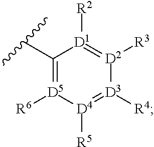

Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
D¹, D², D³, D⁴, D⁵, are independently selected from the group consisting of C and N; preferably C;
R¹ is selected from the group consisting of —(C₁-C₆)alkyl, —(C₃-C₆)cycloalkyl, —CH₂F, —CHF₂, and —CF₃, preferably —(C₁-C₆)alkyl;
R² is selected from the group consisting of H, —(C₁-C₆)alkyl, —CF₃, —C, and —F, preferably H;
R³ is selected from the group consisting of H, —(C₁-C₆)alkyl, —O(C₁-C₆)alkyl, —CF₃, —C, and —F, preferably —O(C₁-C₆)alkyl;
R⁴ is selected from the group consisting of H, —(C₁-C₆)alkyl, —O(C₁-C₆)alkyl, —CF₃, —C, and —F, preferably —O(C₁-C₆)alkyl;
R⁵ is selected from the group consisting of H, —(C₁-C₆)alkyl, —O(C₁-C₆)alkyl, —CF₃, —C, and —F, preferably —O(C₁-C₆)alkyl;
R⁶ is selected from the group consisting of H, —(C₁-C₆)alkyl, —CF₃, —C, and —F, preferably H; two groups of R², R³, R⁴, R⁵ and R⁶ in vicinal position may be part of a ring selected from

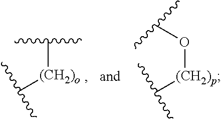

is an integer between 1 and 4;
p is an integer between 1 and 4;
q is 1 or 0;
R⁷ and R⁸ are independently selected from the group consisting of H, and —(C₁-C₆)alkyl;
R⁷ and R⁸ may form a 3 to 5 membered cycloalkyl
R⁹ is selected from the group consisting of H, and —(C₁-C₆)alkyl;
W is —COOH, or —SO₂Cl.

The acylation may be carried out applying acylation conditions known in the art such as disclosed in Peter G. M. Wuts, Greene's Protective Groups in Organic Chemistry, Fifth Edition, Wiley 2014. In particular, the acylation may be carried out in the presence of HATU, and a weak organic base like NEt₃ and Et₂NiPr in a suitable solvent such as DMF

### Pharmaceutical Compositions

In a further aspect, the present invention provides a pharmaceutical composition comprising a compound as specified above under the heading “Compounds” and one or more pharmaceutically acceptable excipients.

The compounds described in present invention in particular those specified above such as those of formula (I), and/or (II), as well as the compounds of table 1 are preferably administered to a patient in need thereof via a pharmaceutical composition. In one embodiment, the pharmaceutical composition comprises a compound as described above (e.g., having the general formula (I), and/or (II), as well as the compounds of table 1 or a hydrate, solvate, salt, complex, racemic mixture, diastereomer, enantiomer, or tautomer thereof or an isotopically enriched form of any of the foregoing) and one or more pharmaceutically acceptable excipients.

The pharmaceutical composition may be administered to an individual by any route, such as enterally or parenterally.

The expressions “enteral administration” and “administered enterally” as used herein mean that the drug administered is taken up by the stomach and/or the intestine. Examples of enteral administration include oral and rectal administration. The expressions “parenteral administration” and “administered parenterally” as used herein mean modes of administration other than enteral administration, usually by injection or topical application, and include, without limitation, intravenous, intramuscular, intraarterial, intrathecal, intracapsular, intraosseous, intraorbital, intracardiac, intradermal, intraperitoneal, transtracheal, subcutaneous, subcuticular, intraarticular, subcapsular, intracerebral, intracerebroventricular, subarachnoid, intraspinal, epidural and intrasternal administration (such as by injection and/or infusion) as well as topical administration (e.g., epicutaneous, inhalational, or through mucous membranes (such as buccal, sublingual or vaginal)).

The compounds used in to the present invention are generally applied in “pharmaceutically acceptable amounts” and in “pharmaceutically acceptable preparations”. Such compositions may contain salts, buffers, preserving agents, carriers and optionally other therapeutic agents.

The term “excipient” when used herein is intended to indicate all substances in a pharmaceutical composition which are not active ingredients (e.g., which are therapeutically inactive ingredients that do not exhibit any therapeutic effect in the amount/concentration used), such as, e.g., carriers, binders, lubricants, thickeners, surface active agents, preservatives, emulsifiers, buffers, flavoring agents, colorants, or antioxidants.

The compositions described in the present invention may comprise a pharmaceutically acceptable carrier. As used herein, “pharmaceutically acceptable carrier” includes any and all solvents, dispersion media, coatings, isotonic and absorption delaying agents, and the like that are physiologically compatible. The “pharmaceutically acceptable carrier” may be in the form of a solid, semisolid, liquid, or combinations thereof. Preferably, the carrier is suitable for enteral (such as oral) or parenteral administration (such as intravenous, intramuscular, subcutaneous, spinal or epidermal administration (e.g., by injection or infusion)). Depending on the route of administration, the active compound, i.e., the compound used in the present invention, either alone or in combination with one or more additional active compounds, may be coated in a material to protect the active compound(s) from the action of acids and other natural conditions that may inactivate the active compound.

Examples of suitable aqueous and non-aqueous carriers which may be employed in the pharmaceutical compositions used according to the present invention include water (e.g., water for injection), ethanol, polyols (such as glycerol, propylene glycol, polyethylene glycol, and the like), aqueous solutions of a salt, carbohydrate, sugar alcohol, or an amino acid (such as saline or an aqueous amino acid solution), and suitable mixtures and/or buffered forms thereof, vegetable oils (such as olive oil), and injectable organic esters (such as ethyl oleate). Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersions. The use of such media and agents for pharmaceutically active compounds is known in the art. Except insofar as any conventional media or agent is incompatible with the active compound, use thereof in the pharmaceutical compositions used according to the present invention is contemplated.

Additional active compounds can be administered together with, before or after the compound used in the present invention (in particular that specified above such as those of general formula (I), and/or (II), as well as the compounds of table 1 or incorporated into the compositions). In one embodiment, the pharmaceutical composition described herein comprises a compound as described above (e.g., having the general formula (I), and/or (II), as well as the compounds of table 1 or a hydrate, solvate, salt, complex, racemic mixture, diastereomer, enantiomer, or tautomer thereof or an isotopically enriched form of any of the foregoing), at least one additional active compound, and one or more pharmaceutically acceptable excipients.

The pharmaceutical composition may comprise an “additional active compound” (which is not a compound having formula (I), and/or (II), as well as the compounds of table 1 as specified herein) may be selected from any compound which can be used in the treatment of cancer and/or immune diseases. The additional active compound may induce an additive or synergistic therapeutic effect.

The pharmaceutical composition described herein may comprise, in addition to the compound having a structure according to formula (I), and/or (II), or the compounds of table 1 as described above, at least one, e.g., 1, 2, 3, 4, 5, 6, 7 or 8, additional active compounds. According to the present teaching, the at least additional active compound, for example a further anticancer drug, may be formulated together with the compound having a structure according to formula (I), and/or (II), or the compounds of table 1 as described above in a single pharmaceutical composition. Alternatively, the pharmaceutical composition may be structured as kit of parts, wherein the compound having a structure according to formula (I), and/or (II), or the compounds of table 1 is provided in a first formulation and the at least one additional active compound, for example the anticancer drug is provided in a second formulation, i.e., a second pharmaceutical composition. The first and the second pharmaceutical compositions may be combined prior to use. In other words, before administering the pharmaceutical composition, a formulation comprising the additional active compound may be added to the first pharmaceutical composition comprising the compound having a structure according to formula (I), and/or (II), or the compounds of table 1 as described above. Alternatively, the present teaching envisages administering the compound having a structure according to formula (I), and/or (II), or the compounds of table 1 as described above, formulated in a first pharmaceutical composition and administering the at least one additional active compound formulated in a second pharmaceutical composition. The pharmaceutical compositions may be administered concomitantly or in succession. For example, the first pharmaceutical composition may be administered at a first point in time and the second pharmaceutical composition may be administered at a second point in time, wherein the points in time may be separated by, for example, 0, or up to 1, 2, 3, 4, 5 or 10 min, up to 1, 2, 3, 4, 5 or 10 hours, up to 1, 2, 3, 4, 5 or 10 days, up to 1, 2, 3, 4, 5 or 10 weeks, up to 1, 2, 3, 4, 5 or 10 months or up to 1, 2, 3, 4, 5 or 10 years.

The compositions may also contain adjuvants such as preservatives, wetting agents, emulsifying agents, pH buffering agents, and dispersing agents. Prevention of the presence of microorganisms may be ensured by sterilization procedures and/or by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the active compounds, which may be used in a suitable hydrated form, and/or the pharmaceutical compositions used according to the present invention, are formulated into pharmaceutically acceptable dosage forms by conventional methods known to those of skill in the art (cf., e.g., Remington, “The Science and Practice of Pharmacy” edited by Allen, Loyd V., Jr., 22^{nd} edition, Pharmaceutical Sciences, September 2012; Ansel et al., “Pharmaceutical Dosage Forms and Drug Delivery Systems”, 7^{th} edition, Lippincott Williams & Wilkins Publishers, 1999.).

A pharmaceutical composition can be administered by a variety of methods known in the art. As will be appreciated by the skilled artisan, the route and/or mode of administration will vary depending upon the desired results. The pharmaceutical compositions containing one or more active compounds can be prepared with carriers that will protect the one or more active compounds against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such compositions are generally known to those skilled in the art. See, e.g., Sustained and Controlled Release Drug Delivery Systems, J. R. Robinson, ed., Marcel Dekker, Inc., New York, 1978.

To administer a compound used in the present invention by certain routes of administration, it may be necessary to coat the compound with, or co-administer the compound with, a material to prevent its inactivation. For example, the compound may be administered to an individual in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7: 27(1984)).

Pharmaceutical compositions typically are sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

An injectable composition should be sterile and fluid to the extent that the composition is deliverable by syringe. In addition to water, the carrier can be an isotonic buffered saline solution, ethanol, polyol (for example, glycerol, propylene glycol, and liquid polyethylene glycol, and the like), and suitable mixtures thereof. Sterile injectable solutions can be prepared by incorporating the active compound in the required amount in an appropriate solvent with one or a combination of ingredients enumerated above, as required, followed by sterilization microfiltration.

Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individuals to be treated; each unit contains a predetermined quantity of active compound calculated to produce the desired therapeutic effect in association with the required pharmaceutical carrier. The specification for the dosage unit forms used according to the present invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic/pharmaceutical formulations, compositions used according to the present invention include those suitable for enteral administration (such as oral or rectal) or parenteral administration (such as nasal, topical (including vaginal, buccal and sublingual)). The compositions may conveniently be presented in unit dosage form and may be prepared by any methods known in the art of pharmacy. The amount of active ingredient (in particular, the amount of a compound used according to the present invention) which can be combined with a carrier material to produce a pharmaceutical composition (such as a single dosage form) will vary depending upon the individual being treated, and the particular mode of administration. The amount of active ingredient which can be combined with a carrier material to produce a single dosage form will generally be that amount of the composition which produces a therapeutic effect.

Generally, out of 100% (for the pharmaceutical formulations/compositions), the amount of active ingredient (in particular, the amount of the compound used according to the present invention, optionally together with other therapeutically active agents, if present in the pharmaceutical formulations/compositions) will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., a compound used according to the present invention, in a unit dosage form and/or when administered to an individual or used in therapy, may range from about 0.1 mg to about 1000 mg (for example, from about 1 mg to about 500 mg, such as from about 10 mg to about 200 mg) per unit, administration or therapy. In certain embodiments, a suitable amount of such active ingredient may be calculated using the mass or body surface area of the individual, including amounts of between about 1 mg/kg and 10 mg/kg (such as between about 2 mg/kg and 5 mg/kg), or between about 1 mg/m² and about 400 mg/m² (such as between about 3 mg/m² and about 350 mg/m² or between about 10 mg/m² and about 200 mg/m²).

Actual dosage levels of the active ingredients in the pharmaceutical compositions used according to the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds used according to the present invention at levels lower than that required in order to achieve the desired therapeutic effect and gradually increase the dosage until the desired effect is achieved. In general, a suitable daily dose of a composition used according to the present invention will be that amount of the compound which is the lowest dose effective to produce a therapeutic effect. Such an effective dose will generally depend upon the factors described above. It is preferred that administration be oral, intravenous, intramuscular, intraperitoneal, or subcutaneous, preferably administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a compound used according to the present invention to be administered alone, it is preferable to administer the compound as a pharmaceutical formulation/composition.

For oral administration, the pharmaceutical composition used according to the present invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration can be in the form of, for example, solutions, syrups, or suspensions, or can be presented as a dry product for constitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, sorbic acids). The preparations can also contain buffer salts, flavouring, coloring and sweetening agents as deemed appropriate. Preparations for oral administration can be suitably formulated to give controlled release of the pharmaceutical composition of the invention.

In one embodiment, the compound is orally administered in a concentration of at most 100 mg/kg body weight (such as at most 50 mg/kg body weight, at most 40 mg/kg body weight, at most 30 mg/kg body weight, at most 20 mg/kg body weight, at most 10 mg/kg body weight, at most 5 mg/kg body weight, at most 4 mg/kg body weight, at most 3 mg/kg body weight, at most 2 mg/kg body weight, at most 1 mg/kg body weight).

In one embodiment, the compound is parenterally administered (e.g., intravenously, intramuscularly, or subcutaneously), in a concentration of at most 10 mg/kg body weight (such as at most 5 mg/kg body weight, at most 4 mg/kg body weight, at most 3 mg/kg body weight, at most 2 mg/kg body weight, at most 1 mg/kg body weight, at most 0.5 mg/kg body weight, at most 0.4 mg/kg body weight, at most 0.3 mg/kg body weight, at most 0.2 mg/kg body weight, at most 0.1 mg/kg body weight).

The pharmaceutical composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

The pharmaceutical composition used according to the present invention can be formulated for parenteral administration by injection, for example, by bolus injection or continuous infusion. In one embodiment, the compounds or compositions used according to the present invention may be administered by slow continuous infusion over a long period, such as more than 24 hours, in order to reduce toxic side effects. The administration may also be performed by continuous infusion over a period of from 2 to 24 hours, such as of from 2 to 12 hours. Such regimen may be repeated one or more times as necessary, for example, after 6 months or 12 months.

In yet another embodiment, the compounds or compositions used according to the present invention are administered by maintenance therapy, such as, e.g., once a week for a period of 6 months or more.

Formulations for injection can be presented in units dosage form (e.g., in phial, in multi-dose container), and with an added preservative. The pharmaceutical composition used according to the present invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Compositions used according to the present invention which are suitable for vaginal administration also include pessaries, tampons, creams, gels, pastes, foams or spray formulations containing such carriers as are known in the art to be appropriate. Dosage forms for the topical or transdermal administration of compositions used according to the present invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

In one embodiment, the compounds used according to the present invention are formulated in liposomes. In a more preferred embodiment, the liposomes include a targeting moiety. In a most preferred embodiment, the compounds in the liposomes are delivered by bolus injection to a site proximal to the desired area. Such liposome-based composition should be fluid to the extent that easy syringability exists, should be stable under the conditions of manufacture and storage and should be preserved against the contaminating action of microorganisms such as bacteria and fungi.

A “therapeutically effective dosage” for therapy/treatment can be measured by objective responses which can either be complete or partial. A complete response (CR) is defined as no clinical, radiological or other evidence of a condition, disorder or disease. A partial response (PR) results from a reduction in disease of greater than 50%. Median time to progression is a measure that characterizes the durability of the objective tumor response.

A “therapeutically effective dosage” for therapy/treatment can also be measured by its ability to stabilize the progression of a condition, disorder or disease. Alternatively, the properties of a compound described in the present invention can be evaluated by examining the ability of the compound in appropriate animal model systems known to the skilled practitioner. A therapeutically effective amount of a compound used according to the present invention can cure, heal, alleviate, relieve, alter, remedy, ameliorate, improve or affect the condition, disorder or disease or the symptoms of the condition, disorder or disease or the predisposition toward the condition, disorder or disease in an individual. One of ordinary skill in the art would be able to determine such amounts based on such factors as the individual's size, the severity of the individual's symptoms, and the particular composition or route of administration selected.

The pharmaceutical composition used according to the invention can also, if desired, be presented in a pack, or dispenser device which can contain one or more unit dosage forms containing the active compound. The pack can for example comprise metal or plastic foil, such as blister pack. The pack or dispenser device can be accompanied with instruction for administration.

The pharmaceutical composition used according to the invention can be administered as sole active agent or can be administered in combination with other therapeutically and/or cosmetically active agents.

### Therapeutic Applications

The compounds according to general formula (I), and/or (II), the compounds of table 1 or a hydrate, solvate, salt, complex, racemic mixture, diastereomer, enantiomer, or tautomer thereof or an isotopically enriched form of any of the foregoing, or a pharmaceutical composition as described above may be used for the treatment of cancer.

The cancer is preferably a cancer expressing ferroptosis suppressor protein-1 (FSP1).

In search of mechanisms that determine resistance versus sensitivity to ferroptosis, a form of iron-dependent cell death, a genetic suppressor screen was performed and apoptosis inducing factor mitochondria-associated 2 (AIFM2) was indentified as a novel “anti-ferroptotic” gene¹⁶. Since it has been shown that AIFM2 (despite its name) does not play any substantial role in apoptosis regulation, it was proposed to rename this oxidoreductase as ferroptosis suppressor protein-1 (FSP1). Overexpression of FSP1 in glutathione peroxidase 4 (GPX4) knockout cells (which inevitably die due to rapid and widespread ferroptosis¹⁹) and in wildtype tumor cells treated with the ferroptosis-inducing tool compound and GPX4 inhibitor (1S, 3R)-RSL-3 conferred unprecedented resistance towards ferroptosis. FSP1 expression has been further detected across a large panel of cancer cell lines which may serve as a biomarker of ferroptosis resistance predicting the ferroptotic response independent of cellular glutathione metabolism and GPX4 activity¹⁶. Initial in vivo studies using the compounds of the present invention in tumor-bearing mice provided proof-of-concept that they impair tumor growth.

More preferably, the cancer is selected from the group consisting of prostate cancer, leukemia (such as acute myeloid leukemia, acute lymphocytic leukemia, chronic myeloid leukemia), liver cancer, breast cancer, hepatocellular carcinoma, cholangiocarcinoma, glioblastoma, uveal melanoma, adrenocortical cancer, thymoma, head and neck squamous cell carcinoma, kidney cancer (such as kidney clear cell carcinoma, renal cell carcinoma), lymphoma (such as lymphoid neoplasm diffuse large B-cell lymphoma, non-Hodgkin lymphoma), pancreatic adenocarcinoma, gallbladder cancer, myeloma, gastric cancer, brain cancer (such as glioblastoma, medulloblastoma, glioma), skin cancer, colon/colorectal cancer, bile duct cancer, neuroblastoma, bone cancer (such as Ewing's sarcoma), and lung cancer (such as small cell lung cancer, non small cell lung cancer, mesothelioma).

Most preferably selected from the group consisting of prostate cancer, leukemia (such as acute myeloid leukemia), kidney cancer (such as kidney clear cell carcinoma, renal cell carcinoma), breast cancer, hepatocellular carcinoma, cholangiocarcinoma, glioblastoma and lung cancer (such as small cell lung cancer, non small cell lung cancer, mesothelioma).

As disclosed in^{16,18,20,21} these cancer cells express FSP1.

The Cancer Genome Atlas (TCGA) and Gene Expression Interactive Analysis (GEPIA 2): cancer types, which show a worse prognosis and/or a higher expression of FSP1 than normal tissues according to database analysis has been performed using The Cancer Genome Atlas (TCGA) and Gene Expression Interactive Analysis (GEPIA 2) are as follows: uveal melanoma, adrenocortical cancer, thymoma, head and neck squamous cell carcinoma, cholangiocarcinoma, kidney clear cell carcinoma, acute myeloid leukemia, lymphoid neoplasm diffuse large B-cell lymphoma, pancreatic adenocarcinoma.

### The Cancer Genome Atlas (TCGA):

https://www.cancer.qov/about-nci/organization/ccq/research/structural-genomics/tcqa.
The results shown here are based upon data generated by the TCGA Research Network:
https://www.cancer.qov/tcqa.

### Gene Expression Interactive Analysis (GEPIA 2):

http://qepia2.cancer-pku.cn/#index
Tang, Z., Kang, B., Li, C., Chen, T., Zhang, Z. (2019). GEPIA2: an enhanced web server for large-scale expression profiling and interactive analysis. Nucleic Acids Res 47, 556-560.

Analysis of the cancer dependency map (DeμMap; https://depmap.org/portal/, https://depmap.org/portal/qene/AIFM2?tab=dependency)) revealed that these are cancer types that have a negative CRISPR Chronos (DeμMap 22Q2) score. Indications with a correlation coefficient of below −0.25 have been selected, meaning there is a potential dependency of the respective cancer cell line on FSP1. The indications here are: Leukemia, Gallbladder Cancer, Lymphoma, Myeloma, Gastric Cancer, Brain Cancer, Lung Cancer, Myeloma, Skin Cancer, Colon/Colorectal Cancer, Bile Duct Cancer, Neuroblastoma, Bone Cancer, Kidney Cancer, Prostate Cancer.

A better understanding of the present invention and of its advantages will be obtained from the following examples, offered for illustrative purposes only. The examples are not intended to limit the scope of the present invention in any way.

### Examples of the Invention

### 1. Synthesis

### 1.1 General Methods and Materials:

All reactions were monitored by TLC with 0.25 mm E. Merck precoated silica gel plates (60 F₂₅₄) and Waters liquid chromatography-mass spectroscopy (LCMS). LC-MS spectra were recorded on a Waters Acquity I class UPLC system using the following system [solvent A: acetonitrile, solvent B: 0.1% formic in water or solvent A: acetonitrile, solvent B: 0.1% ammonia in water or solvent A: acetonitrile, solvent B: 0.1% TFA in water. Formic acid and ammonia or TFA were used as HPLC grade. All the separations were performed at ambient temperatures.

Reverse phase HPLC was performed on a Waters HPLC system using following system [solvent A: acetonitrile, solvent B: 0.1% NH₃ in water] or [solvent A: acetonitrile, solvent B: 0.1% TFA in water]. Ammonia was used as HPLC grade. All the separations were performed at ambient temperatures. For analytical RP-HPLC analysis [Interchim: Acquity BEH C18 (2.1×100 mm, 1.7 um)], the flow rate was 0.4 ml·min⁻¹; injection volume: 10 μL, detection wavelengths: 220 nm and 254 nm. The following gradient was used: 0.01 min 90% B, over 8 min to 10% B, 4 min 10% B.

Purification of reaction products was carried out by column chromatography using commercially available silica or flash chromatography using Combiflash Rf with Teledyne Isco RediSep Rf High Performance Gold or Silicycle SiliaSep High Performance columns (40, 80, or 120 g). The purity of all final compounds was over 95% and was analysed with Waters LCMS system.

¹H NMR spectra were recorded on Varion 400 MHz spectrometers and are reported in ppm with the solvent resonance employed as the internal standard [CDCl₃ at 7.26 ppm, DMSO-d₆ at 2.50 ppm]. Peaks are reported as (s=singlet, d=doublet, t=triplet, q=quartet, m=multiplet or unresolved, br s=broad signal, coupling constant(s) in Hz, integration).

Table of abbreviations

| | |
|---|---|
| Ac₂O | acetic anhydride |
| Boc₂O | di-tert-butyl dicarbonate |
| cHex | cyclohexane |
| CV | column value |
| d | doublet (NMR) |
| d | days |
| dd | doublet of doublets |
| DAPI | 4′,6-diamidino-2-phenylindole |
| DAST | diethylaminosulfur trifluoride |
| DCM | dichloromethane |
| DIPEA | N,N-diethylisopropyl amine |
| DMEM | Dulbecco's Modified Eagle Medium |
| DMF | N,N-dimethylformamide |
| DMSO | dimethyl sulfoxide |
| DMP | Dess-Martin periodinane |
| EDC HCl | 1-Ethyl-3-(3-dimethylaminopropyl)carbodiimid |
| equiv. | equivalents |
| Et₂O | diethyl ether |
| EtOAc | ethyl acetate |
| FBS | Fetal bovine serum |
| FITC | Fluoresceine |
| g | gram |
| h | hours |
| H | proton |
| HCl | hydrochloric acid |
| HEPES | 2-[4-(2-hydroxyethyl)piperazin-1-yl]ethane-1-sulfonic acid |
| H₂O | water |
| HOBT | 1-hydroxybenzotriazol |
| Hz | Hertz |
| IPA | iso-propanol |
| J | scalar ¹H-¹H coupling constant |
| K₂CO₃ | potassium carbonate |
| KOH | potassium hydroxide |
| LC-MS | liquid chromatography - mass spectrometry |
| LDH | lactate dehydrogenase |
| LPS | Lipopolysaccharide |
| m | multiplet |
| M | molar |
| mAU | milli absorption units |
| Me | methyl |
| MeCN | acetonitrile |
| MeOH | methanol |
| mg | milli gram |
| MHz | mega Hertz |
| min | minutes |
| μw | microwave |
| N₂ | nitrogen |
| NADH | Nicotinamide Adenine Dinucleotide |
| NADPH | Nicotinamide adenine dinucleotide phosphate |
| NaH | sodium hydride |
| NaHCO₃ | sodium bicarbonate |
| NaOH | sodium hydroxide |
| Na₂SO₄ | sodium sulfate |
| NBS | N-bromo succinimide |
| NCS | N-chloro succinimid |
| NMR | nuclear magnetic resonance |
| PBS | Phosphate buffered saline |
| PCC | Pyridinium chlorochromate |
| PEG | Polyethylenglycole |
| PVDF | Polyvinylidenfluorid |
| quant. | quantitative |
| R_{f} | retention factor (TLC) |
| rt | room temperature |
| s | singlet |
| SiO₂ | silica |
| TCFH | tetramethylchloroformamidinium hexafluorophosphate |
| TFA | Trifluoro acetic acid |
| THF | Tetrahydrofuran |
| TLC | thin layer chromatography |
| TRIS | Tris(hydroxymethyl)aminomethane |

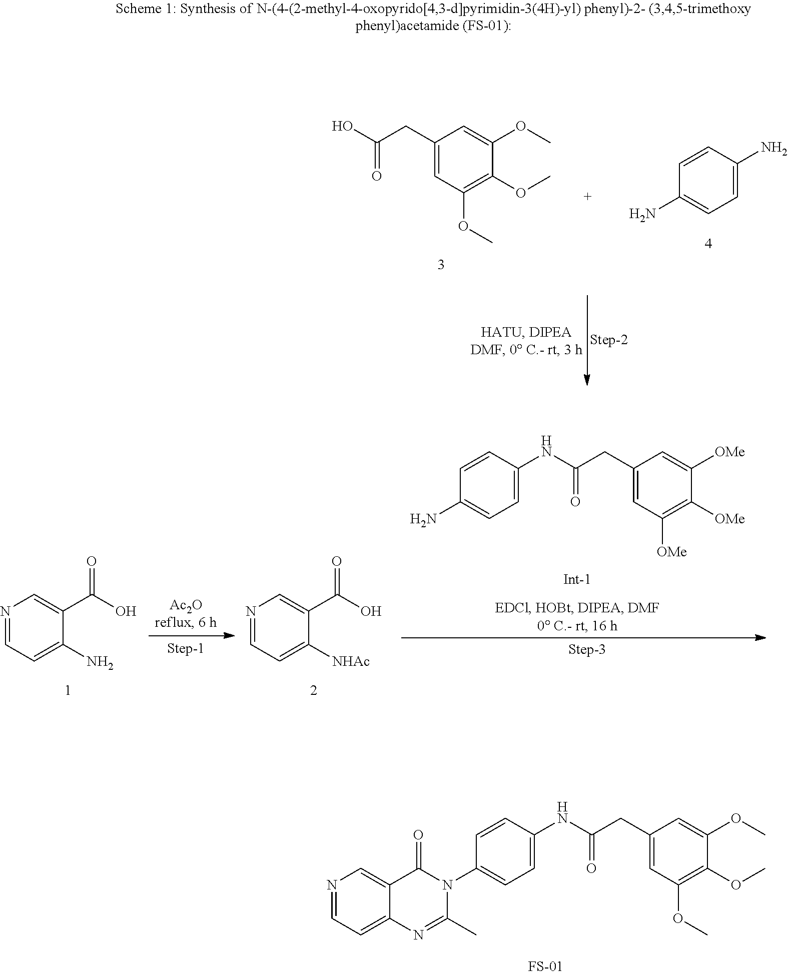 [SMILES: COc1cc(CC(=O)O)cc(OC)c1OC ; Nc1ccc(N)cc1 ; COc1cc(CC(=O)Nc2ccc(N)cc2)cc(OC)c1OC ; Nc1ccncc1C(=O)O ; CC(=O)Nc1ccncc1C(=O)O ; COc1cc(CC(=O)Nc2ccc(-n3c(C)nc4ccncc4c3=O)cc2)cc(OC)c1OC]

### Step-1:

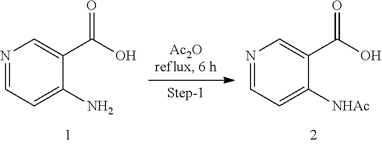 [SMILES: Nc1ccncc1C(=O)O ; CC(=O)Nc1ccncc1C(=O)O]

1.3 Synthesis of 4-acetamidoisonicotinic acid (2): A mixture of 4-aminonicotinic acid (1) (1.0 g, 7.299 mmol, 1 equiv) and acetic anhydride (6 mL) was stirred at room temperature for 15 minutes. The reaction mixture was then refluxed for 6 h. The reaction was monitored by TLC, after completion of reaction, the excess of acetic anhydride was removed under reduced pressure. The crude product was diluted with water (20 mL) and extracted with ethyl acetate (2×50 mL), separated the organic layer and washed with brine solution (20 mL). The combined organic layer was dried over anhydrous Na₂SO₄, filtered and concentrated under reduced pressure to give 4-acetamidoisonicotinic acid (2) (1.1 g, yield: 83%) as a yellow solid. The product was used for next step without further purification.

LCMS: m/z=180.99 [M+H]+, 75.02% (0.87 min).

¹H-NMR (400 MHz, DMSO-d₆) δ ppm: 8.94 (s, 1H), 8.76 (d, J=4.8 Hz, 1H), 7.95 (d, J=4.8 Hz, 1H), 2.43 (s, 3H).

### Step-2:

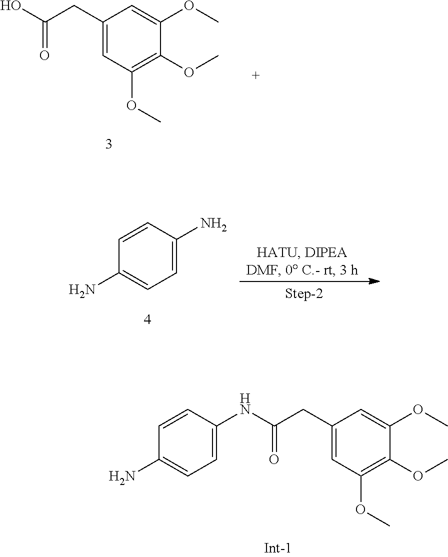 [SMILES: COc1cc(CC(=O)O)cc(OC)c1OC ; Nc1ccc(N)cc1 ; COc1cc(CC(=O)Nc2ccc(N)cc2)cc(OC)c1OC]

1.4 Synthesis of N-(4-aminophenyl)-2-(3,4,5-trimethoxyphenyl)acetamide (Int-1): To a stirred solution of compound 3 (500 mg, 2.252 mmol, 1 equiv) and compound 4 (240 mg, 2.252 mmol, 1 equiv) in DMF (5 mL) was added DIPEA (1.1 mL, 6.756 mmol) and HATU (1.02 g, 3.378 mmol, 1.5 equiv) at 0° C. The reaction mixture was then allowed to room temperature and stirred for 3 h. The reaction was monitored by TLC, after completion of reaction, the reaction mixture was diluted with cold water (20 mL) and extracted with ethyl acetate (2×25 mL), separated the organic layer, washed with brine solution (20 mL). The combined organic layer was dried over anhydrous Na₂SO₄, filtered and concentrated under reduced pressure. The residue was purified by column chromatography using a silica gel (100:200 mesh) and compound was eluted using 12% EtOAc in hexane to afford Int-1 (650 mg, yield: 46%) as a brown gummy liquid.

LCMS: m/z=317.59 [M+H]+, 98.73% (0.88 min).

### Step-3:

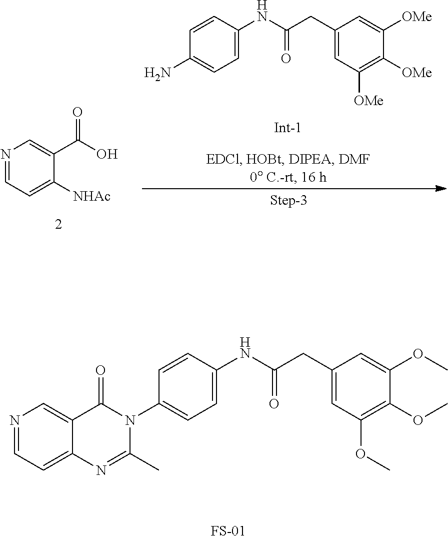 [SMILES: COc1cc(CC(=O)Nc2ccc(N)cc2)cc(OC)c1OC ; CC(=O)Nc1ccncc1C(=O)O ; COc1cc(CC(=O)Nc2ccc(-n3c(C)nc4ccncc4c3=O)cc2)cc(OC)c1OC ; "FS-01"]

1.5 Synthesis of N-(4-(2-methyl-4-oxopyrido[4,3-d]pyrimidin-3(4H)-yl)phenyl)-2-(3,4,5-trimethoxyphenyl)acetamide (FS-01): To a stirred solution of 4-acetamidonicotinic acid (2) (264 mg, 0.833 mmol, 1 equiv) in DMF (2 mL) was added EDC. HCl (191 mg, 0.999 mmol, 1.2 equiv) and HOBt (135 mg, 0.999 mmol, 1.3 equiv) at 0° C. The reaction mixture was stirred for 10 min. Then added a solution of Int-1 (150 mg 0.833 mmol, 1.0 equiv) in DMF (1 mL) at 0° C. The reaction mixture was allowed to room temperature and stirred for 16 h. After complete of reaction monitored by TLC, the reaction mixture was quenched with water (20 mL) and extracted with ethyl acetate (2×25 mL), separated the organic layer and washed with brine solution (15 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-01 (40 mg, yield: 11%) as a white solid.

LCMS: m/z=461.33 [M+H]+, 97.93% (2.11 min).

¹H NMR (400 MHz, DMSO-d₆) δ ppm: 10.38 (s, 1H), 9.05 (s, 1H), 8.65 (d, J=5.2 Hz, 1H), 7.93 (d, J=5.2 Hz, 1H), 7.77 (d, J=9.2 Hz, 2H), 7.38 (d, J=9.2 Hz, 2H), 6.67 (s, 2H), 3.77 (s, 6H), 3.64 (s, 3H), 3.61 (s, 2H), 2.17 (s, 3H).

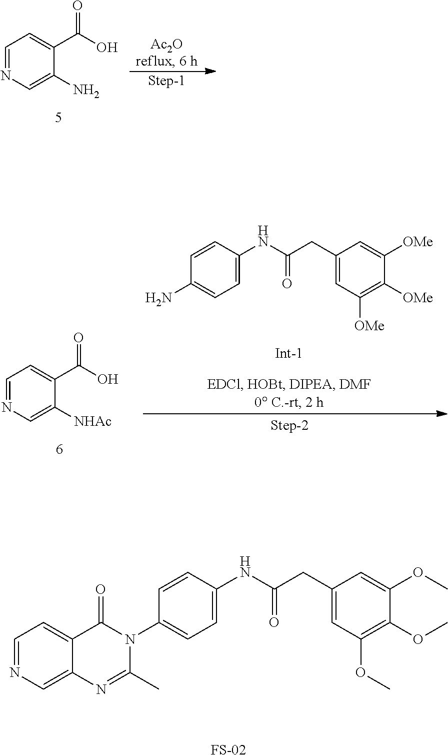 [SMILES: Nc1cnccc1C(=O)O ; COc1cc(CC(=O)Nc2ccc(N)cc2)cc(OC)c1OC ; CC(=O)Nc1cnccc1C(=O)O ; COc1cc(CC(=O)Nc2ccc(-n3c(C)nc4cnccc4c3=O)cc2)cc(OC)c1OC ; "FS-02"]

### Step-1:

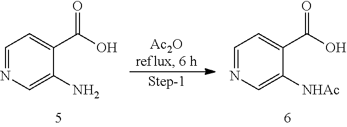 [SMILES: Nc1cnccc1C(=O)O ; CC(=O)Nc1cnccc1C(=O)O]

1.6 Synthesis of 3-acetamidoisonicotinic acid (6): A mixture of 3-aminonicotinic acid 5 (1.0 g, 7.299 mmol, 1 equiv) and acetic anhydride (6 mL) was stirred at room temperature for 15 minutes. The reaction mixture was then refluxed for 6 h. The reaction was monitored by TLC, after completion of reaction, the excess of acetic anhydride removed under reduced pressure. The reaction mixture was diluted with water (20 mL) and extracted with ethyl acetate (2×50 mL), separated the organic layer and washed with brine solution (20 mL). The combined organic layer was dried over anhydrous Na₂SO₄, filtered and concentrated under reduced pressure to give 3-acetamidoisonicotinic acid (6) (900 mg, yield: 69%) as a white solid. The product was used for next step without further purification.

LCMS: m/z=181.03 [M+H]⁺, 81.95% (0.32 min).

¹H-NMR (400 MHz, DMSO-d₆) δ ppm: 12.0 (s, 1H), 11.30 (s, 1H), 9.23 (s, 1H), 8.93 (d, J=5.6 Hz, 1H), 7.51 (d, J=5.6 Hz, 1H), 2.44 (s, 3H).

### Step-2:

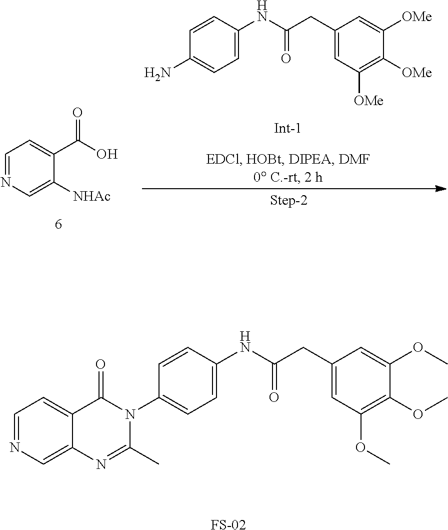 [SMILES: COc1cc(CC(=O)Nc2ccc(N)cc2)cc(OC)c1OC ; CC(=O)Nc1cnccc1C(=O)O ; COc1cc(CC(=O)Nc2ccc(-n3c(C)nc4cnccc4c3=O)cc2)cc(OC)c1OC ; "FS-02"]

1.7 Synthesis of N-(4-(2-methyl-4-oxopyrido[3,4-d]pyrimidin-3(4H)-yl) phenyl)-2-(3,4,5-trimethoxyphenyl)acetamide (FS-02): To a stirred solution of 3-acetamidonicotinic acid (6) (200 mg, 1.111 mmol, 1 equiv) in DMF (2 mL) was added EDC. HCl (191 mg, 1.333 mmol, 1.2 equiv) and HOBt (135 mg, 1.333 mmol, 1.2 equiv) at 0° C. The reaction mixture was stirred for 10 min. Then added a solution of Int-1 (351 mg, 1.111 mmol, 1 equiv) in DMF (1 mL) at 0° C. The reaction mixture was allowed to room temperature and stirred for 16 h. After complete of reaction was monitored by TLC, the reaction mixture was quenched with water (20 mL) and extracted with ethyl acetate (2×25 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-02 (40 mg, yield: 7.8%) as a white solid.

LCMS: m/z=461.38 [M+H]⁺, 95.11% (2.02 min),

¹H NMR (400 MHz, DMSO-d₆) δ ppm: 10.37 (s, 1H), 9.24 (s, 1H), 8.84 (d, J=5.6 Hz, 1H), 7.77 (d, J=8.8 Hz, 2H), 7.56 (d, J=5.6 Hz, 1H), 7.39 (d, J=8.8 Hz, 2H), 6.67 (s, 2H), 3.77 (s, 6H), 3.64 (s, 3H), 3.61 (S, 2H), 2.16 (s, 3H).

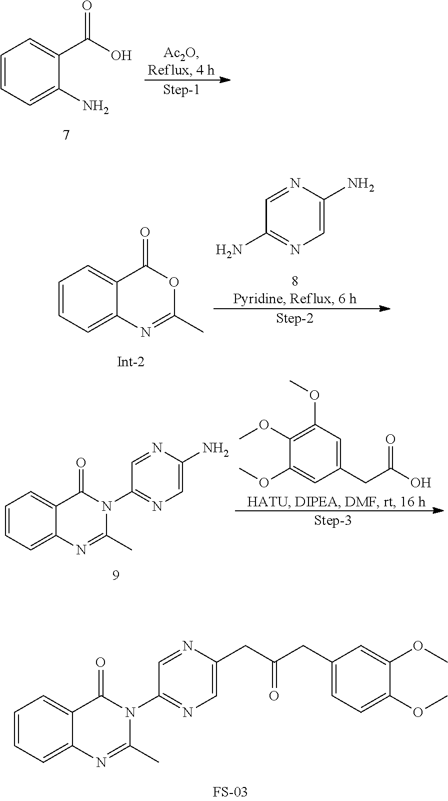

### Step-1:

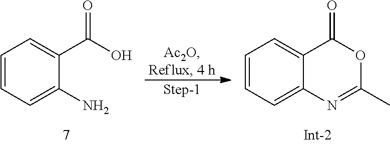

1.7 Synthesis of 2-methyl-4H-benzo[d][1,3]oxazin-4-one (Int-2): A mixture of 2-aminobenzoic acid (7) (5.0 g, 36.496 mmol, 1 equiv) and acetic anhydride (30 mL) was refluxed for 4 h. The reaction mixture was cooled to room temperature and the excess of acetic anhydride was removed under reduced pressure. The crude reaction mixture was diluted with water (50 mL) and extracted with ethyl acetate (2×100 mL), separated the organic layer and washed with brine solution (50 mL). The combined organic layer was dried over anhydrous Na₂SO₄, filtered and concentrated under reduced pressure to afford Int-2 (3.9 g yield: 55%) as an off-white solid. The product was directly used for next step without further purification.

LCMS: m/z=162.04 [M+H]⁺, 95.75% (1.154 min).

### Step-2:

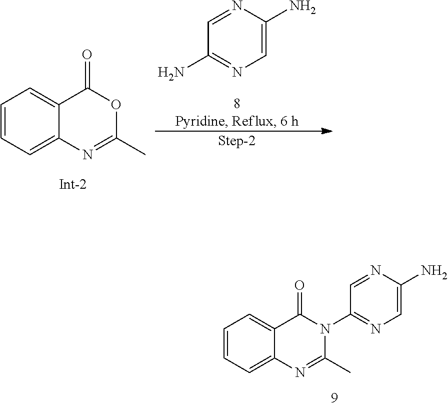 [SMILES: Cc1nc2ccccc2c(=O)o1 ; Nc1cnc(N)cn1 ; Cc1nc2ccccc2c(=O)n1-c1cnc(N)cn1]

1.9 Synthesis of 3-(5-aminopyrazin-2-yl)-2-methylquinazolin-4(3H)-one (9): To a stirred solution of Int-2 (800 mg, 4.968 mmol, 1 equiv) and pyrazine-2,5-diamine 8 (546 mg, 4.968 mol, 1 equiv) in 25 mL of anhydrous pyridine. The reaction mixture was refluxed for 6 h. The resulting mixture was cooled in ice bath and treated with 10 mL of 1N HCl to form a white precipitated solid, washed with water and dried over air. The crude compound was recrystallized from ethanol to afford compound 9 (400 mg, 26% yield) as a brown solid. The product was directly used for next step without further purification.

### Step-3:

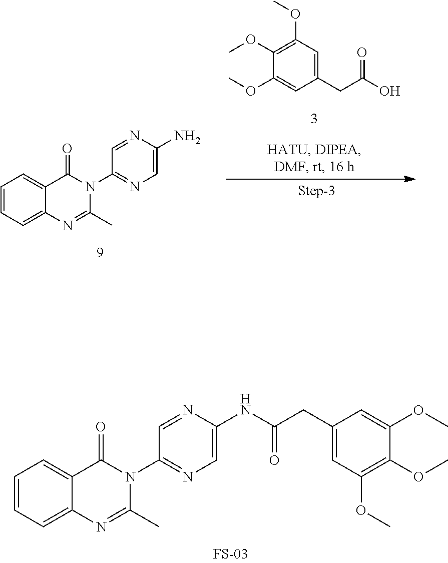 [SMILES: COc1cc(CC(=O)O)cc(OC)c1OC ; Cc1nc2ccccc2c(=O)n1-c1cnc(N)cn1 ; COc1cc(CC(=O)Nc2cnc(-n3c(C)nc4ccccc4c3=O)cn2)cc(OC)c1OC]

1.10 Synthesis of N-(5-(2-methyl-4-oxoquinazolin-3(4H)-yl)pyrazin-2-yl)-2-(3,4,5-trimethoxy phenyl) acetamide (FS-03): To a stirred solution of compound 3 (200 mg, 0.787 mmol, 1 equiv) in DMF (1 mL) was added HATU (360 mg, 0.944 mmol, 1.2 equiv) and DIPEA (0.35 mL, 1.968 mmol, 2.5 equiv) at 0° C. The reaction mixture was stirred for 10 min. Then added a compound 9 (180 mg, 0.787 mmol, 1 equiv) at 0° C. The reaction mixture was allowed to room temperature and stirred for 16 h. After complete of reaction was monitored by TLC, the reaction mixture was quenched with water (20 mL) and extracted with ethyl acetate (2×25 mL), separated the organic layer and washed with brine solution (20 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-03 (8.0 mg, yield: 2.1%) as an off white solid.

LCMS: m/z=462.40 [M+H]⁺, 97.89% (1.30 min).

¹H NMR (400 MHz, CDC₃) 6 ppm: 9.62 (s, 1H), 8.33 (s, 1H) 8.25 (d, J=7.6 Hz, 1H), 8.00 (brs, 1H), 7.79 (t, J=8.4 Hz, 1H), 7.68 (d, J=8.4 Hz, 1H), 7.48 (t, J=7.6 Hz, 1H), 6.55 (s, 2H), 3.90 (s, 6H), 3.88 (s, 3H), 3.78 (S, 2H), 2.26 (s, 3H).

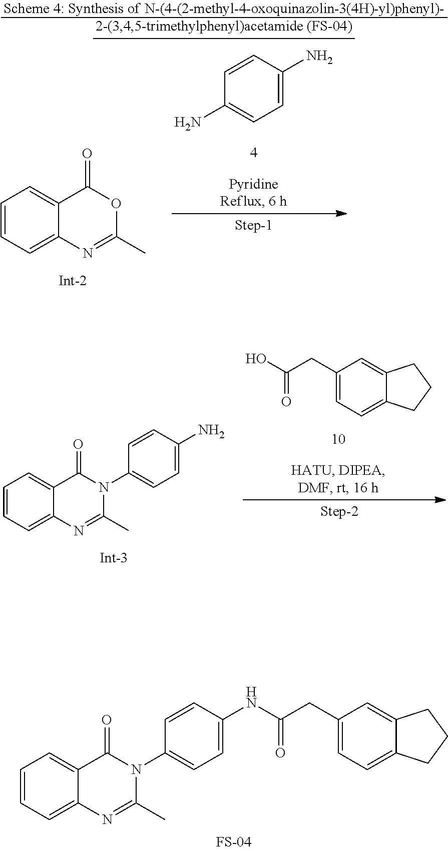 [SMILES: Nc1ccc(N)cc1 ; Cc1nc2ccccc2c(=O)o1 ; O=C(O)Cc1ccc2c(c1)CCC2 ; Cc1nc2ccccc2c(=O)n1-c1ccc(N)cc1 ; Cc1nc2ccccc2c(=O)n1-c1ccc(NC(=O)Cc2ccc3c(c2)CCC3)cc1]

### Step-1:

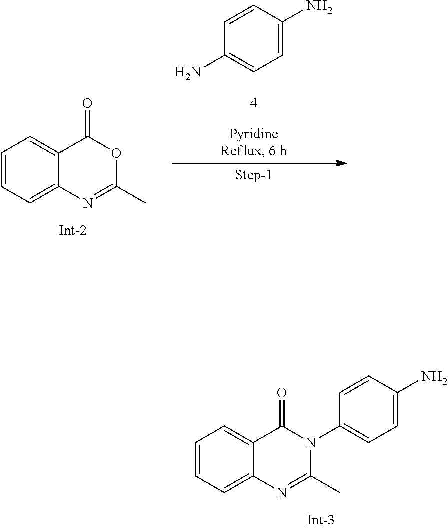 [SMILES: Nc1ccc(N)cc1 ; Cc1nc2ccccc2c(=O)o1 ; Cc1nc2ccccc2c(=O)n1-c1ccc(N)cc1]

1.11 Synthesis of 3-(4-aminophenyl)-2-methylquinazolin-4(3H)-one (Int-3): To a stirred solution of Int-2 (1.5 g, 0.0108 mol, 1 equiv) and p-phenylenediamine 4 (1.40 g, 0.0134 mol, 1.2 equiv) in 15 mL of anhydrous pyridine. The reaction mixture was refluxed for 6 h. The resulting solution was cooled in an ice bath and acidified with 10 mL of dilute hydrochloric acid to form a white precipitated solid and filtered, washed with water, dried over air. The crude residue was recrystallized from ethanol to afford Int-3 (920 mg, 34% yield) as an off-white solid.

LCMS: m/z=252.57 [M+H]⁺, 97.01% (1.04 min).

¹H NMR (400 MHz, DMSO-d₆) δ ppm: 8.16 (d, J=7.6 Hz, 1H), 7.97-7.89 (m, 2H), 7.64 (t, J=7.2 Hz, 1H), 7.50-7.39 (m, 4H), 2.32 (s, 3H).

### Step-2:

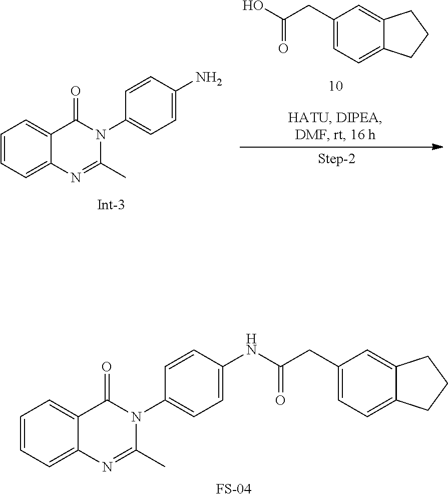 [SMILES: O=C(O)Cc1ccc2c(c1)CCC2 ; Cc1nc2ccccc2c(=O)n1-c1ccc(N)cc1 ; Cc1nc2ccccc2c(=O)n1-c1ccc(NC(=O)Cc2ccc3c(c2)CCC3)cc1]

1.12 Synthesis of N-(4-(2-methyl-4-oxoquinazolin-3(4H)-yl)phenyl)-2-(3,4,5-trimethyl phenyl)acetamide (FS-04): To a stirred solution of compound 10 (100 mg, 0.568 mmol, 1 equiv) and Int-3 (171 mg, 0.681 mmol, 1.2 equiv) in DMF (1 mL) was added HATU (323 mg, 0.852 mmol, 1.5 equiv) followed by DIPEA (0.3 mL, 1.704 mmol, 3.0 equiv) at 0° C. under nitrogen atmosphere. The reaction mixture was allowed to room temperature and stirred for 16 h. The reaction was monitored by TLC, after completion of reaction, the reaction mixture was quenched with water (20 mL) and extracted with ethyl acetate (2×50 mL), separated the organic layer and washed with brine solution (20 mL). The combined organic layer was dried over anhydrous sodium sulphate, filtered and concentrated under reduced pressure. The crude material was purified by C-18 reverse phase column chromatography to afford compound FS-04 (53 mg, yield: 22%) as an off-white solid.

LCMS: m/z=410.41 [M+H]⁺, 99.35% (2.90 min).

¹H NMR (400 MHz, DMSO-d₆) δ ppm: 10.44 (s, 1H), 8.16 (d, J=7.6 Hz, 1H), 7.91 (t, J=7.2 Hz, 1H), 7.83 (d, J=8.4 Hz, 2H), 7.72 (d, J=8.4 Hz, 1H), 7.59 (t, J=7.6 Hz, 1H), 7.43 (d, J=8.4 Hz, 2H), 7.29-725 (m, 2H), 7.18 (d, J=7.6 Hz, 1H), 3.70 (s, 2H), 2.95-2.88 (m, 4H), 2.21 (s, 3H), 2.08 (qt, J=4.0 Hz, 2H).

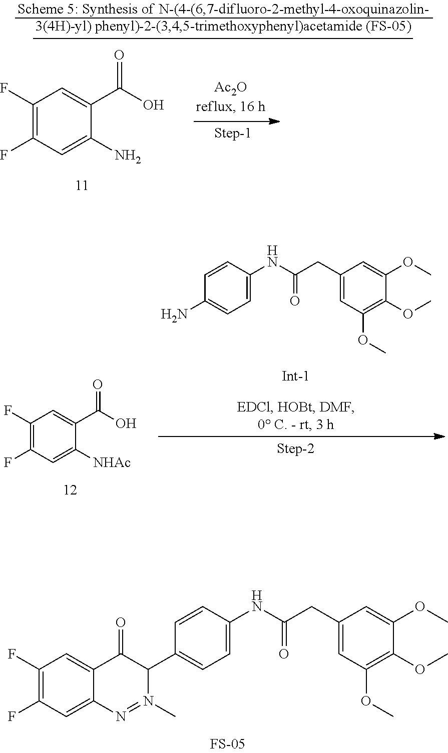

### Step-1:

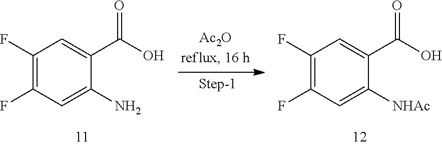 [SMILES: Nc1cc(F)c(F)cc1C(=O)O ; CC(=O)Nc1cc(F)c(F)cc1C(=O)O]

1.13 Synthesis of 2-acetamido-4,5-difluorobenzoic acid (12): In a 50 ml round bottom flask taken compound 11 (500 mg, 2.89 mmol) was added Ac₂O (5 mL) and refluxed for 16 h. The reaction was monitored by TLC, after completion of the reaction, the reaction mixture poured into water (30 mL) and extracted with ethyl acetate (2×50 ml), separated the organic layer and washed with brine solution (20 mL). The combined organic layer was dried over anhydrous Na₂SO₄, filtered and concentrated under reduced pressure. The residue was purified by column chromatography using a silica gel (100-200 mesh) and compound was eluted using 50% EtOAc in hexane to afford compound 12 (400 mg, yield: 61%) as a yellow liquid.

LCMS: m/z=214.12 [M−H]⁻, 99.37% (1.23 min).

### Step-2:

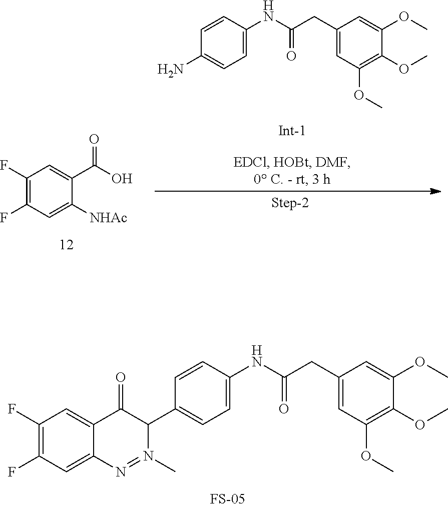

1.14 Synthesis of N-(4-(6,7-difluoro-2-methyl-4-oxoquinazolin-3(4H)-yl)phenyl)-2-(3,4,5-trimethoxyphenyl)acetamide (FS-05): To a stirred solution of compound 14 (300 mg, 1.395 mmol) and Int-1 (529 mg, 1.674 mmol) in DMF (15 mL) was added EDC.HCl (321 mg, 1.674 mmol) followed by HOBt (188 mg, 1.395 mmol) at 0° C. under nitrogen atmosphere. The reaction mixture was allowed to room temperature and stirred for 16 h. The reaction was monitored by TLC, after completion of reaction, the reaction mixture was quenched with water (20 mL) and extracted with ethyl acetate (2×50 mL) separated the organic layer and washed with brine solution (20 mL). The combined organic layer was dried over anhydrous Na₂SO₄, filtered and concentrated under reduced pressure. The crude material was purified by Prep-HPLC to afford compound FS-05 (43 mg, yield: 61%) as a white solid.

LCMS: m/z=496.25 [M+H]⁺, 99.79% (2.66 min).

¹H NMR (400 MHz, DMSO-d₆) δ ppm: 10.36 (s, 1H), 8.01 (t, J=9.2 Hz, 1H), 7.78-7.73 (m, 3H), 7.36 (d, J=8.4 Hz, 2H), 6.67 (s, 2H), 3.77 (S, 6H), 3.64 (S, 3H), 3.61 (s, 2H), 2.12 (s, 3H).

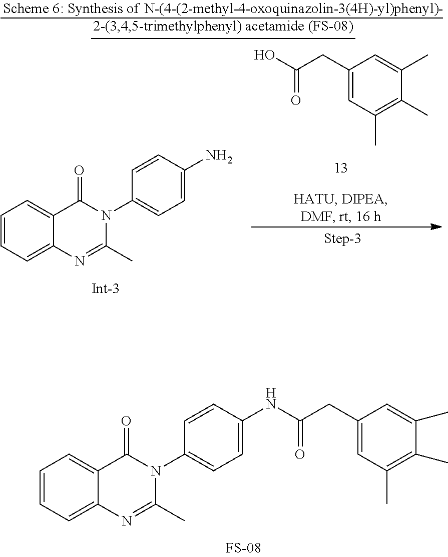 [SMILES: Cc1cc(CC(=O)O)cc(C)c1C ; Cc1nc2ccccc2c(=O)n1-c1ccc(N)cc1 ; "HATU, DIPEA,DMF, rt, 16 h" ; Cc1cc(CC(=O)Nc2ccc(-n3c(C)nc4ccccc4c3=O)cc2)cc(C)c1C]

1.15 Synthesis of N-(4-(2-methyl-4-oxoquinazolin-3(4H)-yl)phenyl)-2-(3,4,5-trimethylphenyl)acetamide (FS-08): To a stirred solution of compound 13 (50 mg, 0.221 mmol, 1.0 eq) and Int-3 (56 mg, 0.212 mmol, 1.0 eq) in DMF (1 mL) was added DIPEA (0.1 mL, 0.663 mmol, 3.0 eq) and HATU (126 mg, 0.331 mmol, 1.5 eq) at 0° C. The reaction mixture was then allowed room temperature and stirred for 16 h. The reaction was monitored by TLC, after completion of reaction diluted with cold water (20 mL) and extracted with ethyl acetate (2×50 mL) separated the organic layer and washed with brine solution (20 mL). The combined organic layer was dried over anhydrous Na₂SO₄ filtered and concentrated under reduced pressure. The crude residue was purified by C-18 reverse phase column chromatography to afford FS-08 (25 mg, yield: 27%) as a white solid.

LCMS: m/z=412.38 [M+H]⁺, 97.99% (2.94 min).

¹H NMR (400 MHz, DMSO-d₆) δ ppm: 10.33 (s, 1H), 8.09 (d, J=8.0 Hz, 1H), 7.83 (t, J=7.2 Hz, 1H), 7.75 (d, J=8.4 Hz, 2H), 7.65 (d, J=8.0 Hz, 1H), 7.51 (t, J=7.6 Hz, 1H), 7.34 (d, J=8.4 Hz, 2H), 6.96 (s, 2H), 3.54 (s, 2H), 2.22 (s, 6H), 2.13 (s, 3H), 2.09 (s, 3H).

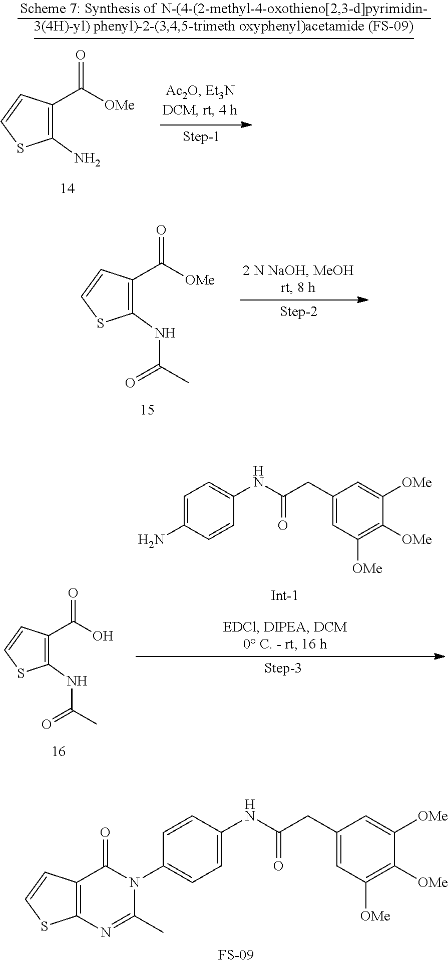 [SMILES: COC(=O)c1ccsc1N ; COC(=O)c1ccsc1NC(C)=O ; COc1cc(CC(=O)Nc2ccc(N)cc2)cc(OC)c1OC ; CC(=O)Nc1sccc1C(=O)O ; COc1cc(CC(=O)Nc2ccc(-n3c(C)nc4sccc4c3=O)cc2)cc(OC)c1OC]

### Step-1:

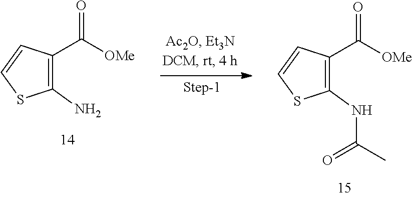

1.16 Synthesis of ethyl 2-acetamidothiophene-3-carboxylate (15): To a stirred solution of ethyl 2-aminothiophene-3-carboxylate (14) (1.0 g, 6.361 mmol, 1 equiv) in dichloromethane (10 mL) was added Et₃N (1.7 mL, 12.738 mmol, 2 equiv) and acetic anhydride (1.2 mL, 12.738 mmol, 2 equiv) at room temperature and stirred for 4 h. The reaction was monitored by TLC, after completion of the reaction, the reaction mixture quenched with saturated NaHCO₃ solution (30 mL) and extracted with dichloromethane (2×50 ml), washed the organics with brine solution (20 mL). The combined organic layer was dried over anhydrous Na₂SO₄, filtered and concentrated under reduced pressure to afford compound 15 (1.0 g, yield: 85%) as a yellow solid. The product was used for next step without further purification.

¹H NMR (400 MHz, DMSO-d₆) δ ppm: 10.79 (s, 1H), 7.16 (d, J=4.0 Hz, 1H), 6.99 (d, J=4.0 Hz, 1H), 3.83 (s, 3H), 2.26 (s, 3H).

### Step-2:

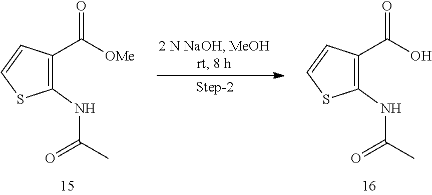

1.17 Synthesis of 2-acetamidothiophene-3-carboxylic acid (16): To a stirred solution of compound 15 (2.5 g, 12.620 mmol, 1 equiv) in methanol (50 mL) was added 2N aq. NaOH solution (30 mL) at 0° C., then allowed to room temperature and stirred for 8 h. After complete of reaction was monitored by TLC, remove the solvent by vacuum. The crude material was diluted with water (10 mL) and acidified with 3N HCl to form a white precipitate solid, filtered and dried over air to afford compound 16 (1.9 g, yield: 82%) as an off-white solid.

¹H NMR (400 MHz, DMSO-d₆) δ ppm: 13.11 (brs, 1H), 10.83 (s, 1H), 7.18 (d, J=4.0 Hz, 1H), 6.91 (d, J=6.0 Hz, 1H), 2.24 (s, 3H) (INT-NY-671-093-01).

### Step-3:

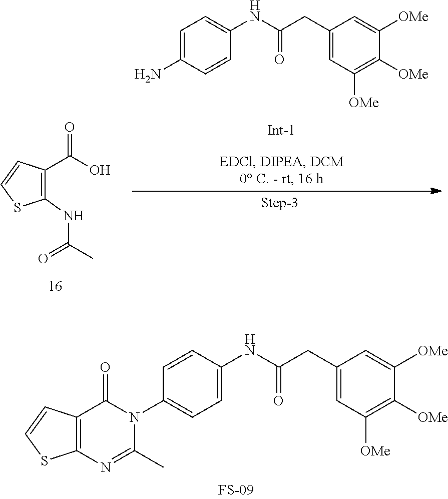 [SMILES: COc1cc(CC(=O)Nc2ccc(N)cc2)cc(OC)c1OC ; CC(=O)Nc1sccc1C(=O)O ; COc1cc(CC(=O)Nc2ccc(-n3c(C)nc4sccc4c3=O)cc2)cc(OC)c1OC]

1.18 Synthesis of N-(4-(2-methyl-4-oxothieno[2,3-d]pyrimidin-3(4H)-yl)phenyl)-2-(3,4,5-trimeth oxyphenyl)acetamide (FS-09): To a stirred solution of compound 16 (150 mg, 0.810 mmol, 1 equiv) in DMF (2 mL) was added EDC.HCl (186 mg, 0.972 mmol, 1.2 equiv) and HOBt (131 mg, 0.972 mmol, 1.2 equiv) at 0° C. The reaction mixture was stirred for 10 min. Then added a solution of Int-1 (256 mg 0.810 mmol, 1 equiv) at 0° C. The reaction mixture was allowed to room temperature and stirred for 16 h. After complete of reaction monitored by TLC, after completion of reaction, the reaction mixture was quenched with water (20 mL) and extracted with ethyl acetate (2×25 mL) and separated the organic layer and washed with brine solution (20 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give 100 mg with 80% purity. Finally purified by prep-HPLC and lyophilisation to afford FS-09 (21 mg, 5.5%) as an off-white solid.

LCMS: m/z=466.16 [M+H]⁺, 99.92% (2.36 min).

¹H NMR (400 MHz, DMSO-d₆) δ ppm: 10.37 (s, 1H), 7.75 (d, J=8.4 Hz, 2H), 7.54 (d, J=6.0 Hz, 1H), 7.37-7.32 (m, 3H), 6.67 (s, 2H), 3.77 (s, 6H), 3.63 (s, 3H), 3.61 (s, 2H), 2.13 (s, 3H).

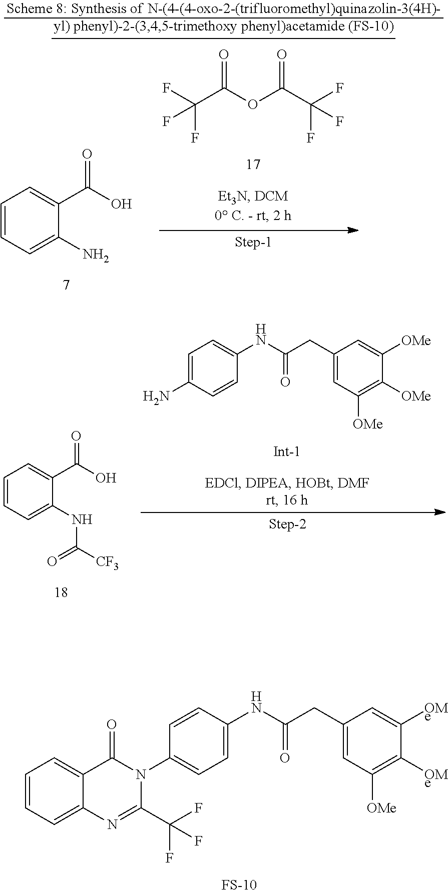

### Step-1:

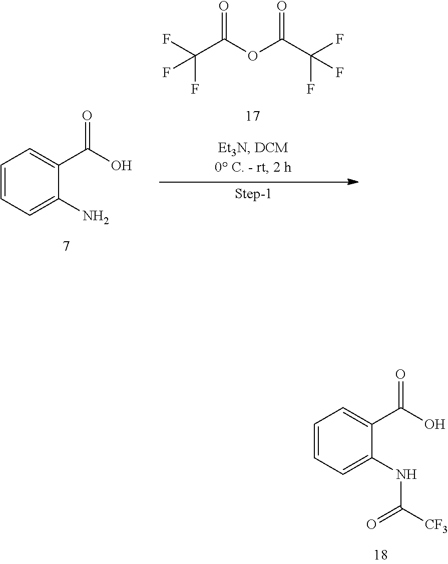 [SMILES: O=C(OC(=O)C(F)(F)F)C(F)(F)F ; Nc1ccccc1C(=O)O ; O=C(O)c1ccccc1NC(=O)C(F)(F)F]

1.19 Synthesis of 2-(2,2,2-trifluoroacetamido)benzoic acid (18): A mixture of 2-aminobenzoic acid (7) (1.0 g, 7.296 mmol, 1 equiv) and trifluoroacetic anhydride 17 (1.37 mL, 14.593 mmol, 2 equiv) was stirred for 15 minutes, then triethylamine (3.0 mL, 21.897 mmol, 3 equiv) was added slowly at 0° C., the reaction mixture was allowed to room temperature and stirred for 2 h. The crude product was diluted with water (20 mL) and extracted with ethyl acetate (2×50 mL), separated the organic layer and washed with brine solution (20 mL). The combined organic layer was dried over anhydrous Na₂SO₄, filtered and concentrated under reduced pressure to give compound 18 (1.1 g, yield: 65%) as a white solid. The product was used for next step without further purification.

### Step-2:

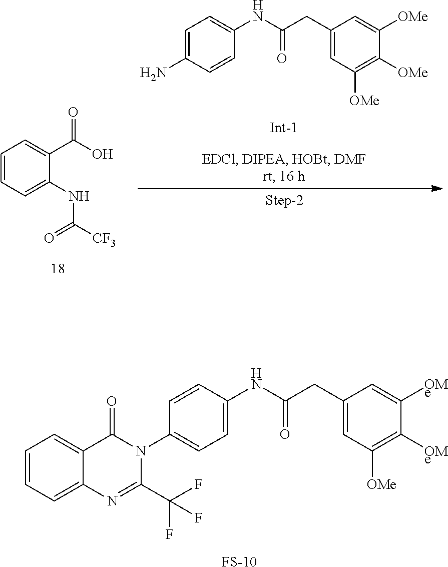

1.20 Synthesis of N-(4-(4-oxo-2-(trifluoromethyl)quinazolin-3(4H)-yl)phenyl)-2-(3,4,5-tri methoxyphenyl)acetamide (FS-10): To a stirred solution of compound 18 (500 mg, 2.145 mmol, 1.0 equiv) in DMF (5 mL) was added EDC.HCl (491 mg, 2.572 mmol, 1.2 equiv) and HOBt (339 mg, 2.575 mmol, 1.2 equiv) and followed by DIPEA (339 mg, 2.575 mmol, 1.2 equiv) at 0° C. The reaction mixture was stirred for 10 min. Then added a solution of Int-1 (678 mg 2.145 mmol, 1 equiv) at 0° C. The reaction mixture was allowed to room temperature and stirred for 16 h. After complete of reaction monitored by TLC, the reaction mixture was quenched with water (20 mL) and extracted with ethyl acetate (2×25 mL) and separated the organic layer and washed with brine solution (20 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilisation gave crude compound. Finally purified by prep-HPLC and lyophilisation to afford FS-10 (28 mg, yield: 3%) as a white solid.

LCMS: m/z=514.39 [M+H]⁺, 96.63% (2.88 min). (INT-NY-709-028-03)

¹H NMR (400 MHz, DMSO-d) 6 ppm: 10.37 (s, 1H), 8.20 (d, J=8.0 Hz, 1H), 7.99-7.96 (m, 1H), 7.90 (d, J=7.6 Hz, 1H), 7.77-7.72 (m, 3H), 7.43 (d, J=8.4 Hz, 2H), 6.67 (s, 2H), 3.77 (s, 6H), 3.63 (s, 3H), 3.61 (s, 2H).

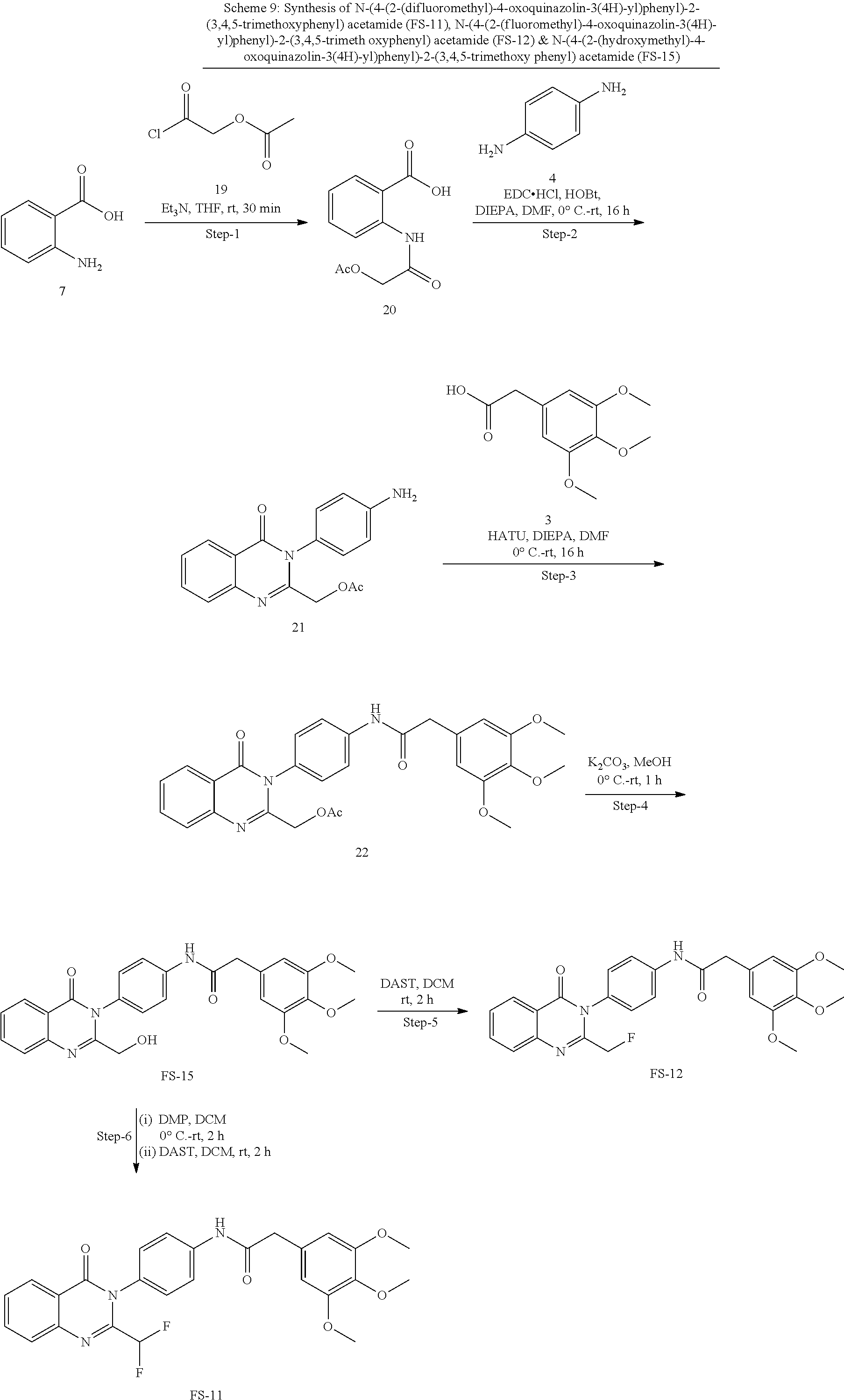

### Step-1:

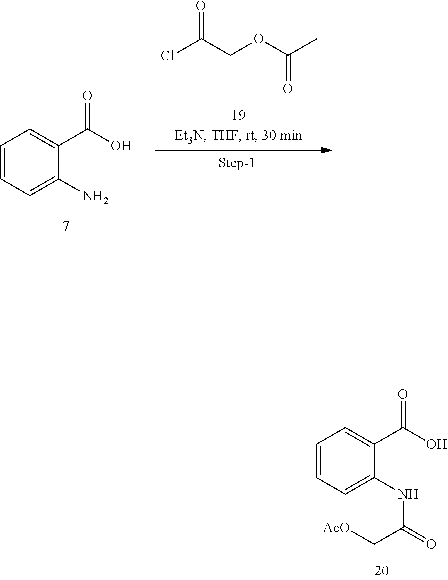

1.21 Synthesis of 2-(2-acetoxyacetamido) benzoic acid (20): To a stirred solution of 2-aminobenzoic acid (7) (5.0 g, 36.496 mmol, 1 equiv) in THF (50 mL) was added triethylamine (15.2 mL, 109.48 mmol, 3 equiv), followed by 2-chloro-2-oxoethyl acetate (19) (7.44 g, 54.744 mmol, 1.5 equiv) the reaction mixture was stirred for 30 minutes at room temperature. The reaction mixture was monitored by TLC, after completion of the reaction solvent evaporated under reduced pressure. The crude material was diluted with water (100 mL) and extracted with ethyl acetate (2×150 mL) and separated the organic layer and washed with brine solution (20 mL). The combined organic layer was dried over anhydrous Na₂SO₄, filtered and concentrated under reduced pressure to afford compound 20 (3.2 g, yield: 37%) as a yellow solid. The product was used for next step without further purification.

### Step-2:

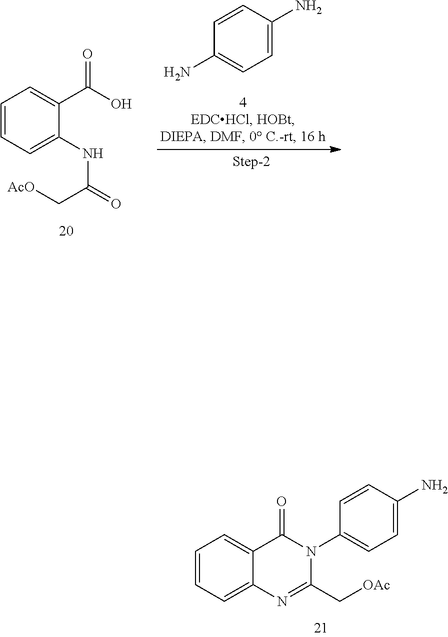 [SMILES: CC(=O)OCC(=O)Nc1ccccc1C(=O)O ; Nc1ccc(N)cc1 ; CC(=O)OCc1nc2ccccc2c(=O)n1-c1ccc(N)cc1]

1.21 Synthesis of (3-(4-aminophenyl)-4-oxo-3,4-dihydroquinazolin-2-yl)methyl acetate (23): To a stirred solution of compound 20 (2.0 g, 8.438 mol, 1.0 equiv) in DMF (10 mL) was added EDC.HCl (1.9 g, 10.126 mmol, 1.2 equiv) and HOBt (1.4 g, 0.010.126 mmol, 1.2 equiv), followed by DIPEA (4 mL, 25.314 mmol, 3.0 eq) at 0° C. The reaction mixture was stirred for 10 min. Then added a solution of compound 4(908 mg 8.438 mmol, 1.0 equiv) at 0° C. The reaction mixture was allowed to room temperature and stirred for 16 h. After complete of reaction monitored by TLC, the reaction mixture was quenched with water (20 mL) and extracted with ethyl acetate (2×25 mL) and separated the organic layer and washed with brine solution (20 mL). The combined organic layer was dried over anhydrous Na₂SO₄, filtered and concentrated under reduced pressure. The residue was purified by column chromatography using a silica gel (100:200 mesh) and compound was eluted using 20% EtOAc in hexane to afford compound 21 (1.8 g, yield: 69%) as a yellow oil.

LCMS: m/z=310.08 [M+H]⁺, 92.75% (1.11 min).

### Step-3:

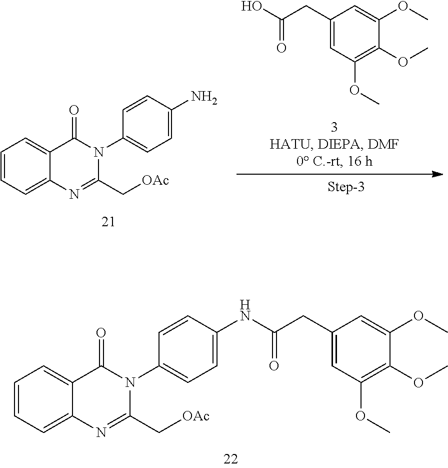 [SMILES: CC(=O)OCc1nc2ccccc2c(=O)n1-c1ccc(N)cc1 ; COc1cc(CC(=O)O)cc(OC)c1OC ; COc1cc(CC(=O)Nc2ccc(-n3c(COC(C)=O)nc4ccccc4c3=O)cc2)cc(OC)c1OC]

1.22 Synthesis of (4-oxo-3-(4-(2-(3,4,5-trimethoxyphenyl)acetamido)phenyl)-3,4-dihydro quinazolin-2-yl)methyl acetate (22): To a stirred solution of compound 3 (300 mg, 1.327 mmol, 1.0 equiv) and compound 21 (410 mg, 1.327 mmol, 1.0 eq) in DMF (3 mL) was added DIPEA (0.6 mL, 3.98 mmol, 3.0 eq) and HATU (750 mg, 1.99 mmol) at 0° C. The reaction mixture was then allowed to stir at room temperature for 16 h. The reaction was monitored by TLC, after completion of reaction, the reaction mixture was diluted with cold water (20 mL) and extracted with ethyl acetate (2×50 mL) and separated the organic layer and washed with brine solution (20 mL). The combined organic layer was dried over anhydrous sodium sulphate, filtered and concentrated under reduced pressure. crude compound was purified by reverse phase C18 column chromatography and lyophilisation to afford compound 22 (210 mg, yield: 30%) as a light brown solid.

LCMS: m/z=518.12 [M+H]⁺, 96.93% (1.33 min).

### Step-4:

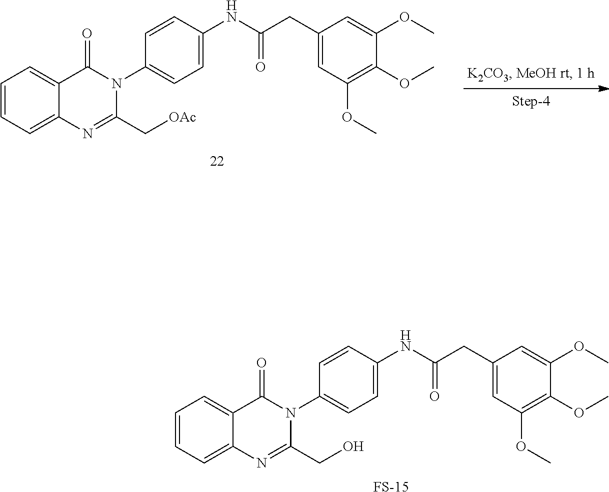

1.23 Synthesis of N-(4-(2-(hydroxymethyl)-4-oxoquinazolin-3(4H)-yl) phenyl)-2-(3,4,5-trimethoxyphenyl) acetamide (FS-15): To a stirred solution of compound 22 (240 mg, 0.4642 mmol 1.0 equiv) in MeOH (4.8 mL), was added K₂CO₃ (161 mg, 1.160 mmol, 2.5 equiv) at room temperature and stirred for 1 h. The reaction was monitored by TLC, after completion of reaction, crude compound was purified by using prep HPLC chromatography 0.01% HCOOH buffer in acetonitrile/water and after lyophilisation gave FS-15 (7.2 mg, Yeild-3%) as a white solid.

LCMS: m/z=476.29 [M+H]⁺, 99.84% (2.20 min), (INT-NY-709-055-02)

¹H NMR (400 MHz, DMSO-d₆) δ ppm: 10.37 (s, 1H), 8.12 (d, J=7.6 Hz, 1H), 7.88 (t, J=8.0 Hz, 1H), 7.79-7.73 (m, 3H), 7.56 (t, J=7.6 Hz, 1H), 7.33 (d, J=8.4 Hz, 2H), 6.68 (s, 2H), 5.19 (t, J=6.0 Hz, 1H), 4.05 (d, J=6.0 Hz, 2H), 3.77 (s, 6H), 3.64 (s, 3H), 3.61 (s, 2H).

### Step-5:

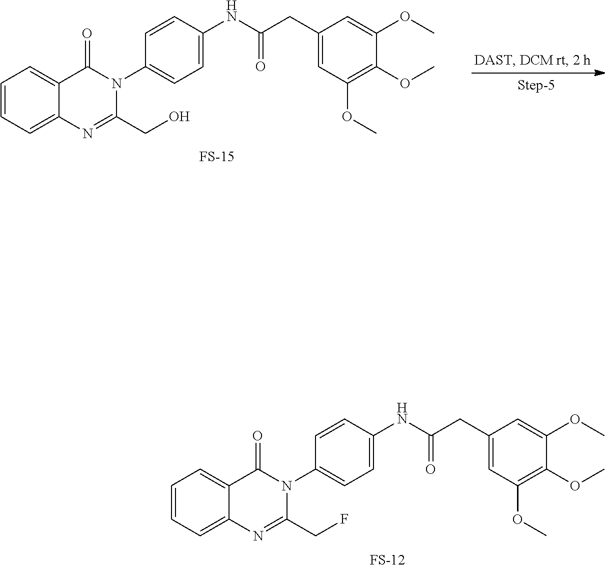

1.24 Synthesis of N-(4-(2-(fluoromethyl)-4-oxoquinazolin-3(4H)-yl)phenyl)-2-(3,4,5-trimeth oxyphenyl) acetamide (FS-12): To a stirred solution of FS-15 (40 mg, 0.084 mmol, 1.0 equiv) in DCM (2.4 mL) was added DAST (0.01 ml, 0.1094 mmol, 1.3 equiv) at 0° C. The reaction mixture was allowed to room temperature and stirred for 2 h. The reaction was monitored by TLC, after completion of reaction, to remove the solvent by vacuum. The residue was purified by RP C18 column using 0.01% HCOOH buffer in acetonitrile/water and after lyophilisation to give FS-12. (8.0 mg, yield: 20%) as an off white solid.

LCMS: m/z=478.10 [M+H]⁺, 98.04% (2.39 min).

¹H NMR (400 MHz, DMSO-d₆) δ ppm: 10.40 (s, 1H), 8.15 (d, J=8.0 Hz, 1H), 7.91 (t, J=7.6 Hz, 1H), 7.81-7.75 (m, 3H), 7.61 (t, J=7.6 Hz, 1H), 7.39 (d, J=8.4 Hz, 2H), 6.68 (s, 2H), 4.97 (d, J=6.0 Hz, 2H), 3.77 (s, 6H), 3.64 (s, 3H), 3.61 (s, 2H).

### Step-6:

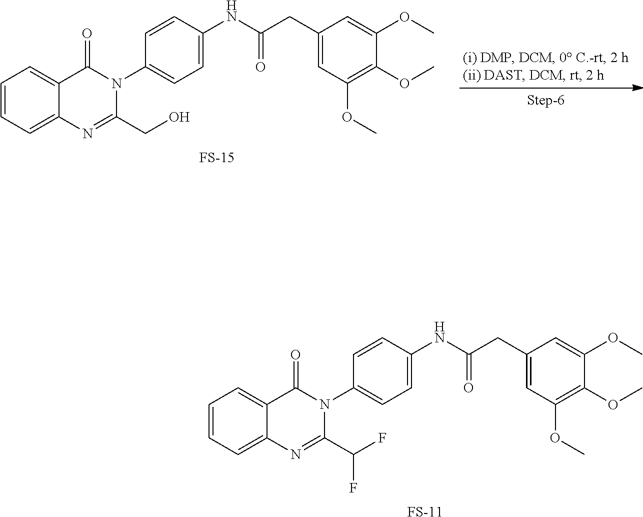

1.25 Synthesis of N-(4-(2-(difluoromethyl)-4-oxoquinazolin-3(4H)-yl)phenyl)-2-(3,4,5-trimethoxyphenyl) acetamide (FS-11): To a stirred solution of FS-15 (500 mg, 1.05 mmol, 1.0 equiv) in dichloromethane (5 mL) was added portion wise Dess-Martin reagent (530 mg, 1.26 mmol, 1.2 eq) at 0° C. After addition was completed the reaction mixture was allowed to room temperature and stirred for 2 h. After DAST (0.1 mL, 169 mmol, 1.0 eq) was added slowly at 0° C. and reaction mixture was allowed to room temperature and stirred for 2 h. The reaction was monitored by TLC, after completion of reaction, to remove the solvent by vacuum. The residue was purified by prep HPLC to afford FS-11 (8.0 mg, yield: 2%) as an off white solid.

LCMS: m/z=518.25 [M+Na]⁺, 98.12% (2.64 min).

¹H NMR (400 MHz, DMSO-d₆) δ ppm: 10.40 (s, 1H), 8.17 (d, J=8.0 Hz, 1H), 7.96 (t, J=8.4 Hz, 1H), 7.85 (d, J=8.0 Hz, 1H), 7.75 (d, J=8.8 Hz, 2H), 7.69 (t, J=8.0 Hz, 1H), ), 7.40 (d, J=8.8 Hz, 2H), 6.68 (s, 2H), 3.77 (s, 6H), 3.64 (s, 3H), 3.61 (s, 2H).

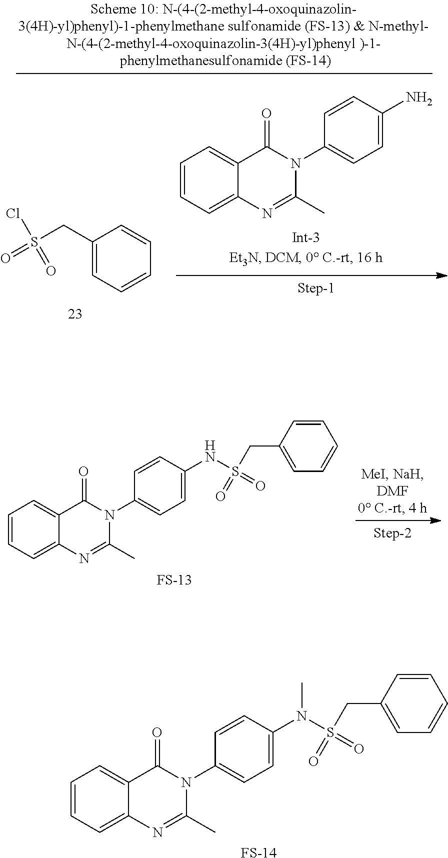

### Step-1:

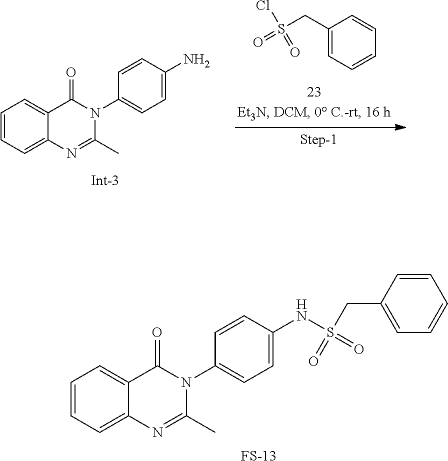

1.26 Synthesis of N-(4-(2-methyl-4-oxoquinazolin-3(4H)-yl)phenyl)-1-phenylmethane sulfonamide (FS-13): To a stirred solution of Int-3 (650 mg, 2.589 mmol, 1.0 equiv) in dichloromethane (10 mL) was added Et₃N (1.5 mL, 10.316 mmol, 4.0 equiv) and compound 23 (550 mg, 18.055 mmol, 1.0 equiv) at 0° C. The reaction mixture was allowed to room temperature and stirred for 16 h. After complete of reaction was monitored by TLC, the reaction mixture was quenched with NaHCO₃ solution (20 mL) and extracted with ethyl acetate (2×25 mL), separate the organic layer and washed with brine solution (20 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilization to afford FS-13 (100 mg, 7.2%) as a white solid.

LCMS: m/z=406.08 [M+H]⁺, 98.60% (2.48 min).

¹H NMR (400 MHz, DMSO-d₆) δ ppm: 10.14 (s, 1H), 8.10 (d, J=7.2 Hz, 1H), 7.87-7.82 (m, 1H), 7.66 (d, J=8.0 Hz, 1H), 7.52 (t, J=7.6 Hz, 1H), 7.38-7.29 (m, 9H), 4.60 (s, 2H), 2.17 (s, 3H).

### Step-2:

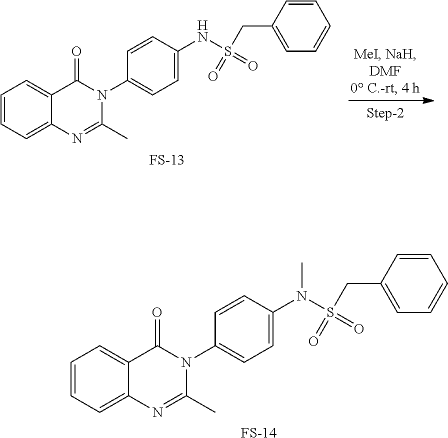

1.27 Synthesis of N-methyl-N-(4-(2-methyl-4-oxoquinazolin-3(4H)-yl)phenyl)-1-phenylmeth-anesulfonamide (FS-14): To a stirred solution of compound FS 13 (35 mg, 0.086 mmol, 1 equiv) in DMF (2 mL) was added NaH (6 mg, 0.129 mmol, 1.5 equiv) and 1M solution of methyl iodide in DMF (0.12 mL, 0.129 mmol, 1.5 equiv) at 0° C. The reaction mixture was allowed to room temperature and stirred for 4 h. After complete of reaction was monitored by TLC, the reaction mixture was quenched with water (20 mL) and extracted with ethyl acetate (2×25 mL), then separated the organic layer, washed with brine solution (20 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilization to afford FS-14 (35 mg, 97%) as a white solid.

LCMS: m/z=420.39 [M+H]⁺, 99.74% (2.71 min).

¹H NMR (400 MHz, DMSO-d₆) δ ppm: 8.09 (d, J=7.6 Hz, 1H), 7.84 (t, J=7.2 Hz, 1H), 7.65 (d, J=7.6 Hz, 1H), 7.52 (t, J=7.6 Hz, 1H), 7.43-7.37 (m, 9H), 4.64 (s, 2H), 3.28 (s, 3H), 2.12 (s, 3H).

### Structures of Target Compounds:

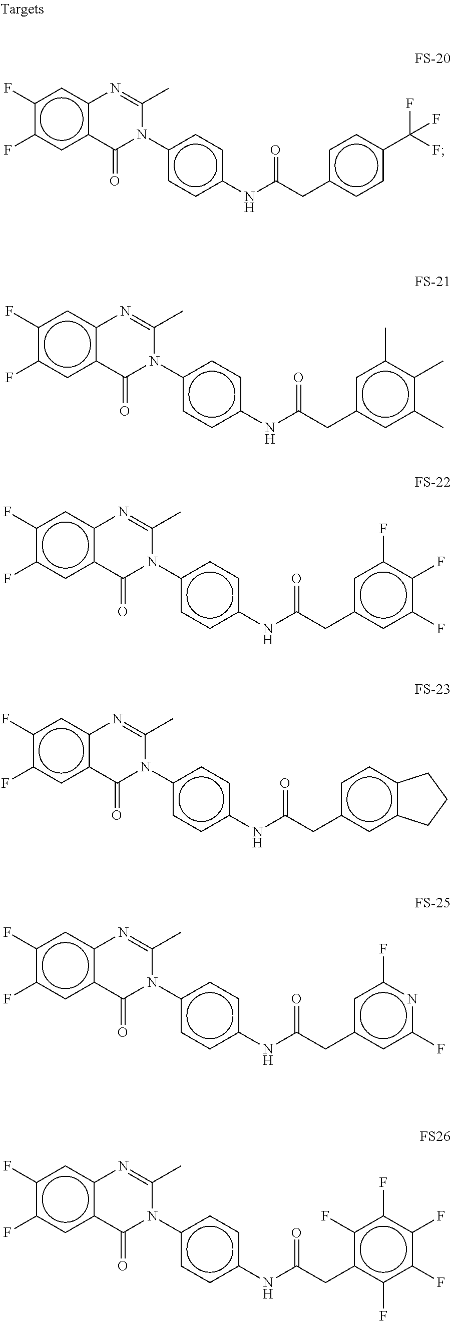

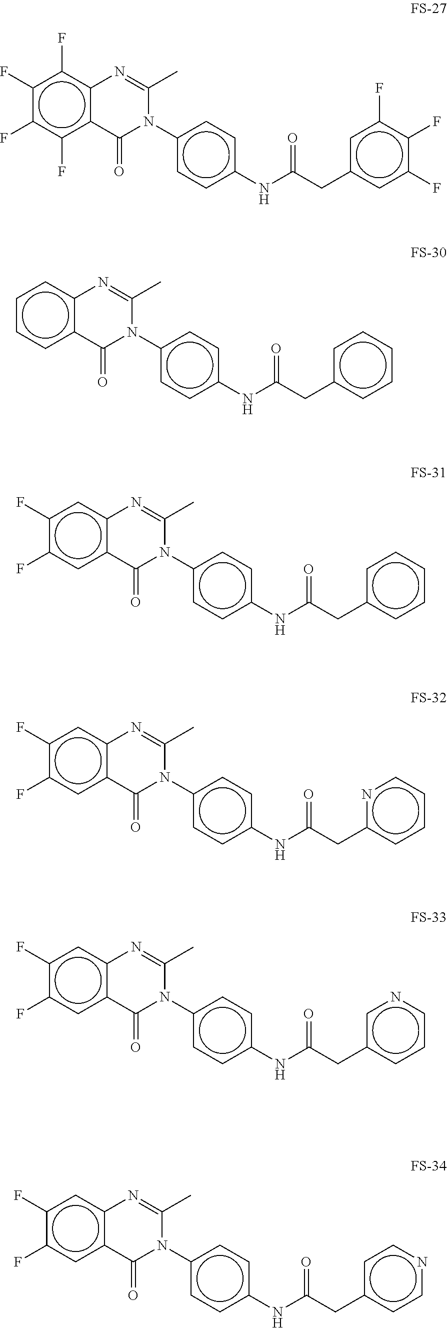

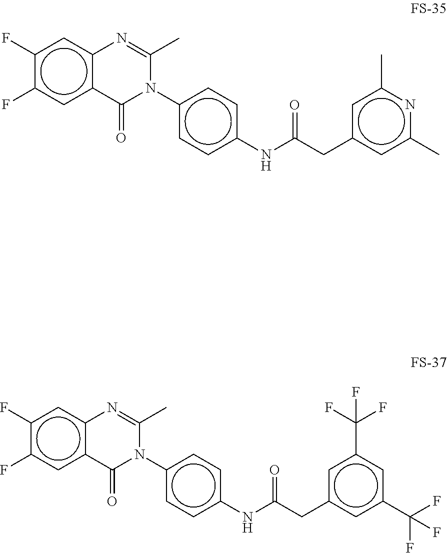

### Scheme 11:

### N-(4-(6,7-difluoro-2-methyl-4-oxoquinazolin-3(4H)-yl)phenyl)-2-(4-(trifluoromethyl)phenyl)acetamide (FS-20)

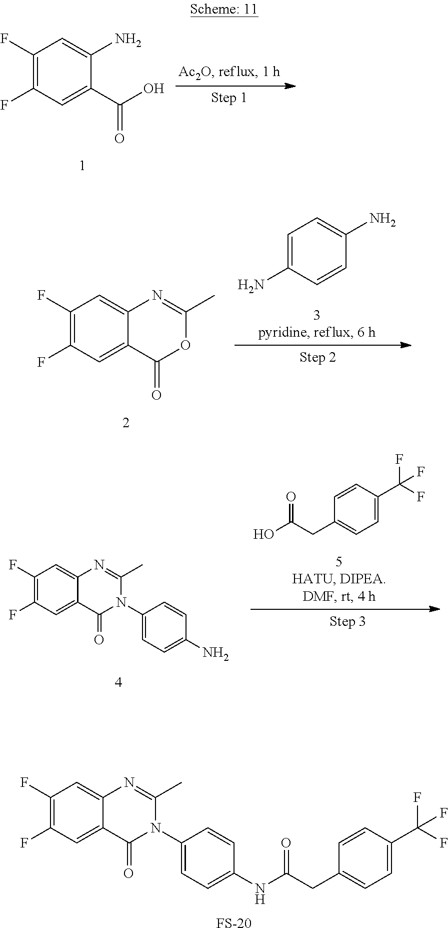

### Step 1

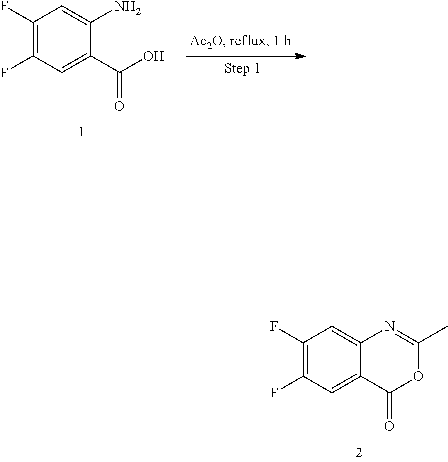

Synthesis of 6,7-difluoro-2-methyl-4H-benzo[d][1,3]oxazin-4-one (2): A mixture of 2-amino-4,5-difluorobenzoic acid (1) (5.0 g, 0.028 mmol, 1 equiv) and Ac₂O (30 mL) was stirred at room temperature for 15 minutes. The reaction mixture was then refluxed for 6 h. The reaction was monitored by TLC, after completion of reaction, the excess of acetic anhydride was removed under reduced pressure. The crude product was diluted with water (50 mL) and extracted with EtOAc (2×50 mL), separated the organic layer and washed with brine solution (20 mL). The organic layer was dried over anhydrous Na₂SO₄, filtered and concentrated under reduced pressure to afford compound (2) (4.2 g, Yield-71%) as an off white solid. The product was used for the next step without further purification.

LCMS: m/z=198.11 [M+H]⁺, 97.33% (1.32 min),

¹H-NMR (400 MHz, DMSO-d₆) δ ppm: 8.14 (t, J=8.8 Hz, 1H), 7.759-7.714 (m, 1H), 2.403 (s, 3H).

### Step-2:

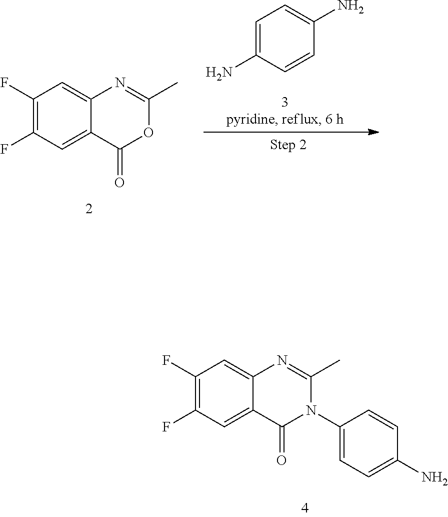 [SMILES: Nc1ccc(N)cc1 ; Cc1nc2cc(F)c(F)cc2c(=O)o1 ; Cc1nc2cc(F)c(F)cc2c(=O)n1-c1ccc(N)cc1]

Synthesis of 3-(4-aminophenyl)-6,7-difluoro-2-methylquinazolin-4(3H)-one (4): To a stirred solution of 6,7-difluoro-2-methyl-4H-benzo[d][1,3]oxazin-4-one (2) (4.0 g, 0.020 mmol, 1 equiv) in 120 mL of anhydrous pyridine was added benzene-1,4-diamine (3) (2.18 g, 0.020 mmol, 1 equiv). The reaction mixture was refluxed for 6 h. The resulting mixture was cooled in ice bath and treated with 50 mL of 1N HCl to form a brown precipitated solid, washed with water and dried over air. The crude compound was recrystallized from ethanol (20 ml) to afford compound (4) (4.2 g, 72% yield) as a brown solid. The product was directly used for next step without further purification.

LCMS: m/z=288.15 [M+H]⁺, 81.01% (1.24 min),

¹H-NMR (400 MHz, DMSO-d₆) δ ppm: 8.0 (t, J=8.8 Hz, 1H), 7.73 (q, J=7.2 Hz, 1H), 6.9 (d, J=8.8 Hz, 2H), 6.65 (d, J=8.8 Hz, 2H), 5.41 (br, s, 2H), 2.14 (s, 3H).

### FS-20:

### Step-3:

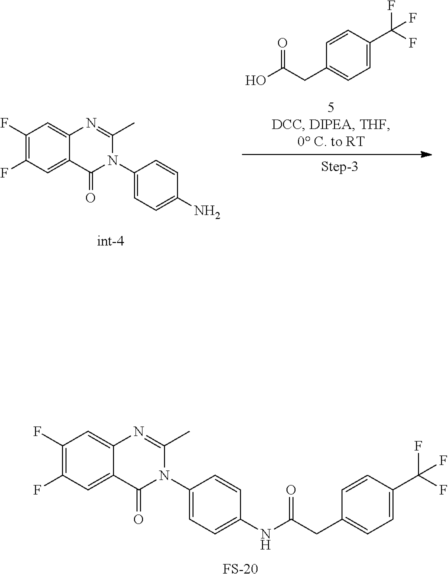

Synthesis of N-(4-(6,7-difluoro-2-methyl-4-oxoquinazolin-3(4H)-yl)phenyl)-2-(4-(trifluoromethyl)phenyl)acetamide (FS-20): To a stirred solution of 2-(4-(trifluoromethyl)phenyl)acetic acid (5) (100 mg, 0.348 mmol, 1 equiv) and 3-(4-aminophenyl)-6,7-difluoro-2-methylquinazolin-4(3H)-one (4) (71 mg, 0.348 mmol, 1 equiv) in DMF (1 mL) was DIPEA (0.2 mL, 1.045 mmol, 4 equiv) followed by DCC (252 mg, 1.225 mmol, 2.5 equiv) at 0° C. under nitrogen atmosphere. The reaction mixture was then allowed to room temperature and stirred for 4 h. The reaction was monitored by TLC, after completion of reaction, the reaction mixture was diluted with cold water (20 mL) and extracted with ethyl acetate (2×25 mL), separated the organic layer, washed with brine solution (20 mL). The separated organic layer was dried over anhydrous Na₂SO₄, filtered and concentrated under reduced pressure. The crude residue was purified by reverse phase C-18 column using 0.01% NH₃ buffer in acetonitrile/water and after lyophilisation, to give FS-20 (35 mg, Yield-21%) as an off white solid.

LCMS: m/z=474.24 [M+H]⁺, 96.59% (3.02 min).

¹H NMR (400 MHz, DMSO-d₆) δ ppm: 10.49 (s, 1H), 8.01 (t, J=9.2 Hz, 1H), 7.69-7.79 (m, 5H), 7.58 (d, J=7.8 Hz, 2H), 7.37 (d, J=8.6 Hz, 2H), 3.83 (s, 2H), 2.13 (s, 3H).

### FS-21:

### Synthesis of N-(4-(6,7-difluoro-2-methyl-4-oxoquinazolin-3(4H)-yl)phenyl)-2-(3,4,5-trimethylphenyl)acetamide(FS21)

### Step-3:

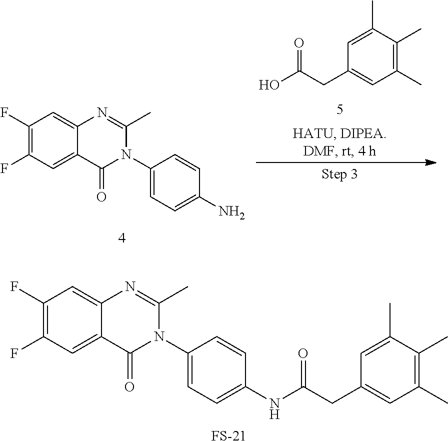

Synthesis of N-(4-(6,7-difluoro-2-methyl-4-oxoquinazolin-3(4H)-yl)phenyl)-2-(3,4,5-trimethylphenyl)acetamide (FS-21): To a stirred solution of 2-(3,4,5-trimethylphenyl)acetic acid (5) (100 mg, 0.561 mmol, 1 equiv) and 3-(4-aminophenyl)-6,7-difluoro-2-methylquinazolin-4(3H)-one (4) (193 mg, 0.674 mmol, 1.2 equiv) in DMF (1 mL), followed by DIPEA (0.3 mL, 0.842 mmol) and HATU (320 mg, 1.685 mmol, 1.5 equiv) was added at 0° C. The reaction mixture was then allowed to stir at room temperature for 4 h. The reaction progress was monitored by TLC, after completion of reaction, the reaction mixture was diluted with cold water (20 mL) and extracted with ethyl acetate (2×25 mL). The separated the organic layer washed with brine solution (20 mL). The organic layer was dried over anhydrous Na₂SO₄, filtered and concentrated under reduced pressure. The crude residue was purified by reverse phase C18 column using 0.01% HCOOH buffer in acetonitrile/water and after lyophilization gave FS-21 (45 mg, Yield-26%) as an off white solid.

LCMS: m/z=448.51 [M+H]⁺, 98.90% (3.21 min).

¹H NMR (400 MHz, DMSO-d₆) δ ppm: 10.34 (s, 1H), 8.02 (t, J=9.2 Hz, 1H), 7.73-7.78 (m, 3H) 7.35 (d, J=8.8 Hz, 2H), 6.95 (br, s, 2H), 3.54 (s, 2H), 2.2 (br, s, 6H), 2.12 (s, 3H), 2.09 (s, 3H).

### FS-22:

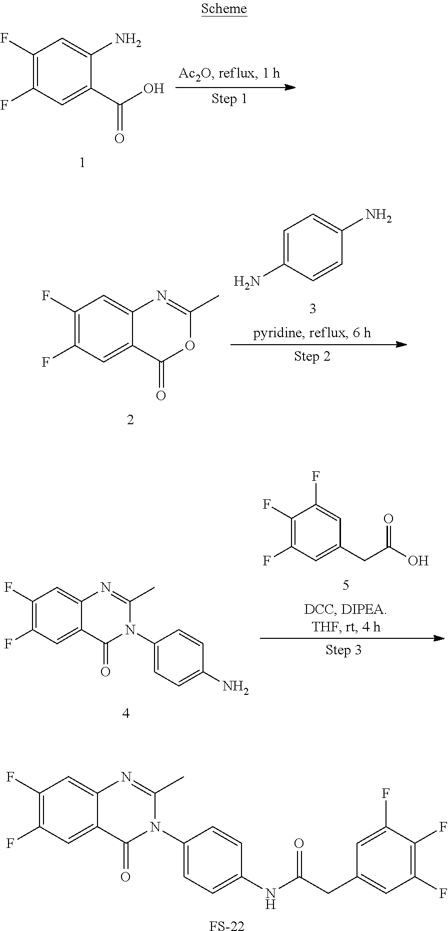

### Step-3:

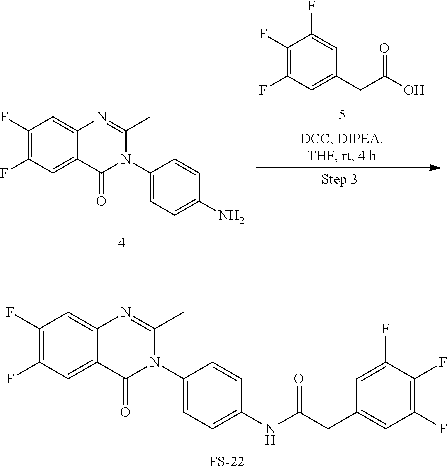

Synthesis of N-(4-(6,7-difluoro-2-methyl-4-oxoquinazolin-3(4H)-yl)phenyl)-2-(3,4,5-trifluorophenyl)acetamide (FS-22): To a stirred solution of 2-(3,4,5-trifluorophenyl)acetic acid (5) (100 mg, 0.348 mmol, 1 equiv) and 3-(4-aminophenyl)-6,7-difluoro-2-methylquinazolin-4(3H)-one (4) (66 mg, 0.348 mmol, 1 equiv) in THF (1 mL), followed by DIPEA (0.27 mL, 1.392 mmol) and DCC (94 mg, 0.871 mmol, 2.5 equiv) was added at 0° C. The reaction mixture was then allowed to room temperature and stirred for 4 h. The reaction was monitored by TLC, after completion of reaction, the reaction mixture was diluted with cold water (20 mL) and extracted with ethyl acetate (2×25 mL). The separated the organic layer, washed with brine solution (20 mL). The organic layer was dried over anhydrous Na₂SO₄, filtered and concentrated under reduced pressure. The crude residue was purified by reverse phase C18 column using 0.01% NH₃ buffer in acetonitrile/water and after lyophilization gave FS-22 (38 mg, Yield-23%) as a brown solid.

LCMS: m/z=460.25 [M+H]⁺, 97.32% (2.94 min).

¹H NMR (400 MHz, DMSO-d₆) δ ppm: 10.45 (s, 1.0H), 8.01 (t, J=9.2 Hz, 1H), 7.7 (d, J=8.8 Hz, 3H), 7.29-7.386 (m, 4H), 3.69 (br, s, 2H), 2.07 (m, 3H).

### FS-23:

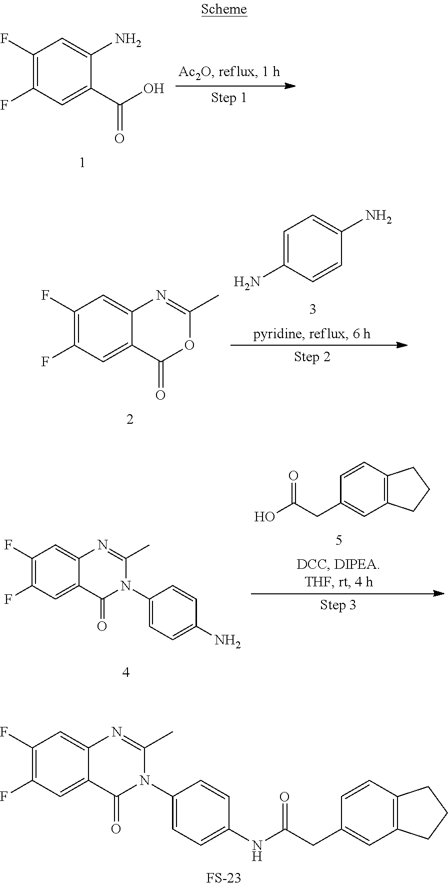

### Step-3:

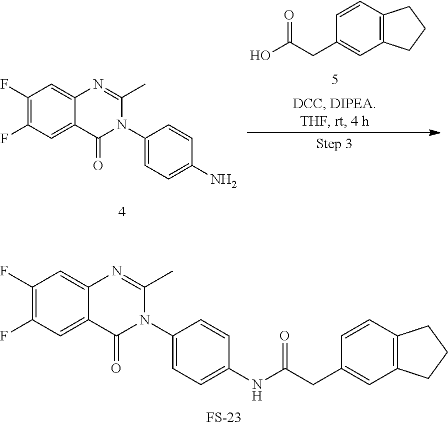

Synthesis of N-(4-(6,7-difluoro-2-methyl-4-oxoquinazolin-3(4H)-yl)phenyl)-2-(2,3-dihydro-1H-inden-5-yl)acetamide (FS-23): To a stirred solution of 2-(2,3-dihydro-1H-inden-5-yl)acetic acid (5) (100 mg, 0.568 mmol, 1 equiv) and 3-(4-aminophenyl)-6,7-difluoro-2-methylquinazolin-4(3H)-one (4) (195 mg, 0.681 mmol, 1.2 equiv) in DMF (1 mL), followed by DIPEA (0.3 mL, 1.704 mmol) and HATU (323 mg, 0.852 mmol, 1.5 equiv) was added at 0° C. The reaction mixture was then allowed to room temperature and stirred for 4 h. The reaction was monitored by TLC, after completion of reaction, the reaction mixture was diluted with cold water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by reverse phase C18 column using 0.01% HCOOH buffer in acetonitrile/water and after lyophilization gave FS-23 (40 mg, Yield-25%) as a white solid.

LCMS: m/z=446.38 [M+H]⁺, 97.92% (3.16 min).

¹H NMR (400 MHz, DMSO-d₆) δ ppm: 10.38 (s, 1H), 8.01 (t, J=8.8 Hz, 1H), 7.74-7.78 (m, 3.0H), 7.35 (d, J=8.8 Hz, 2H), 7.16-7.22 (m, 1H), 7.10 (d, J=7.6 Hz, 1H), 7.08 (d, J=7.6 Hz, 1H), 3.62 (s, 2H), 2.80-2.86 (m, 4H), 2.07 (s, 3H), 2.0 (t, J=7.2 Hz, 2H).

### FS-25:

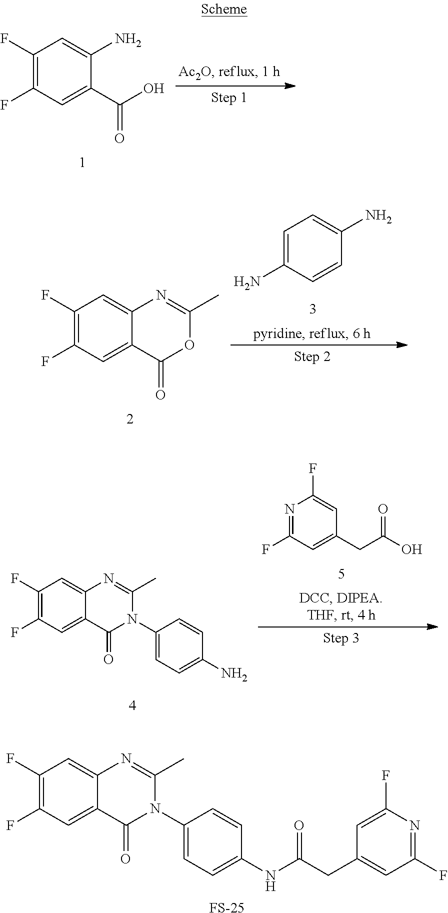

### Step-3:

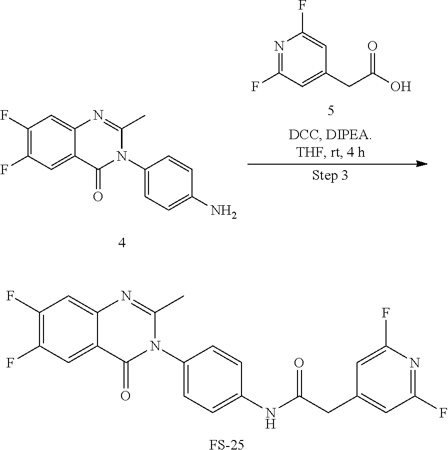

N-(4-(6,7-difluoro-2-methyl-4-oxoquinazolin-3(4H)-yl)phenyl)-2-(2,6-difluoropyridin-4-yl)acetamide (FS-22): To a stirred solution of compound 5 (100 mg, 0.578 mmol, 1 equiv) and 3-(4-aminophenyl)-6,7-difluoro-2-methylquinazolin-4(3H)-one (4) (165 mg, 0.348 mmol, 1 equiv) in THF (1 mL), DIPEA (0.4 mL, 2.312 mmol) and DCC (269 mg, 1.308 mmol, 2.5 equiv) was added at 0° C. The reaction mixture was then allowed to room temperature and stirred for 4 h. The reaction was monitored by TLC, after completion of reaction, the reaction mixture was diluted with cold water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by reverse phase C18 column using 0.01% NH₃ buffer in acetonitrile/water and after lyophilisation, to give FS-25 (23 mg, Yield-15%) as an off white solid.

LCMS: m/z=443.27 [M+H]⁺, 99.00% (2.77 min).

¹H NMR (400 MHz, DMSO-d₆) δ ppm: 10.5 (s, 1.0H), 8.02 (t, J=9.2 Hz, 1H), 7.7 (d, J=9.2 Hz, 3H), 7.38 (d, J=8.8 Hz, 2H), 7.1 (s, 2H), 3.87 (br, s, 2H), 2.0 (s, 3H).

### FS-26:

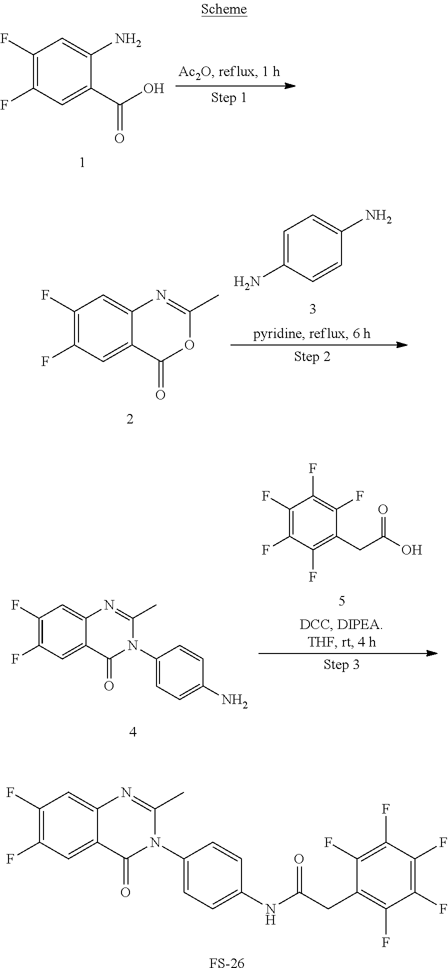

### Step-3:

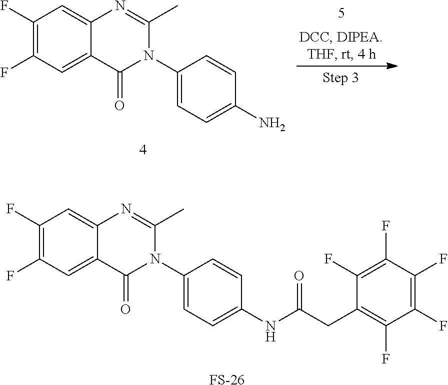 [SMILES: Cc1nc2cc(F)c(F)cc2c(=O)n1-c1ccc(N)cc1 ; Cc1nc2cc(F)c(F)cc2c(=O)n1-c1ccc(NC(=O)Cc2c(F)c(F)c(F)c(F)c2F)cc1 ; "FS-26"]

Synthesis of N-(4-(6,7-difluoro-2-methyl-4-oxoquinazolin-3(4H)-yl)phenyl)-2-(perfluorophenyl)acetamide (FS-26): To a stirred solution of 2-(perfluorophenyl)acetic acid (5) (100 mg, 0.442 mmol, 1 equiv) and 3-(4-aminophenyl)-6,7-difluoro-2-methylquinazolin-4(3H)-one (4) (126 mg, 0.442 mmol, 1 equiv) in THF (1 mL), DIPEA (0.3 mL, 1.768 mmol) and DCC (227 mg, 1.106 mmol, 2.5 equiv) was added at 0° C. The reaction mixture was then allowed to room temperature and stirred for 4 h. The reaction was monitored by TLC, after completion of reaction, the reaction mixture was diluted with cold water (20 mL) and extracted with ethyl acetate (2×25 mL), separated the organic layer, washed with brine solution (20 mL). The combined organic layer was dried over anhydrous Na₂SO₄, filtered and concentrated under reduced pressure. The crude residue was purified by reverse phase C18 column using 0.01% NH₃ buffer in acetonitrile/water and after lyophilisation, to give FS-26 (35 mg, Yield-20%) as an off white solid.

LCMS: m/z=496.23 [M+H]⁺, 95.21% (3.05 min).

¹H NMR (400 MHz, DMSO-d₆) δ ppm: 10.63 (s, 1H), 8.02 (t, J=9.3 Hz, 1H), 7.72-7.78 (m, 3H), 7.38 (d, J=8.8 Hz, 2H), 3.95 (s, 2H), 2.13 (s, 3H).

### FS-31:

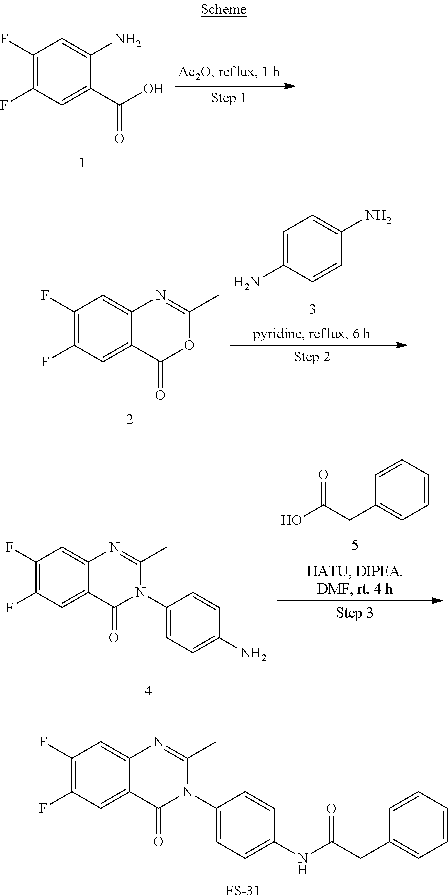

### Step-3:

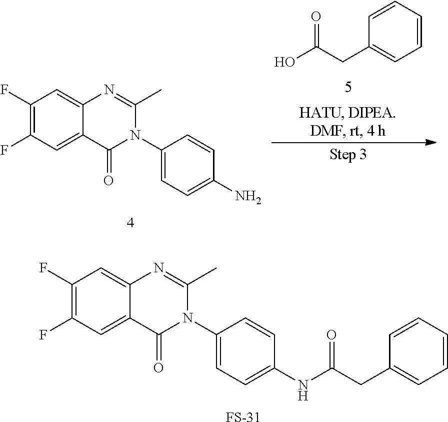 [SMILES: O=C(O)Cc1ccccc1 ; Cc1nc2cc(F)c(F)cc2c(=O)n1-c1ccc(N)cc1 ; Cc1nc2cc(F)c(F)cc2c(=O)n1-c1ccc(NC(=O)Cc2ccccc2)cc1 ; "FS-31"]

Synthesis of N-(4-(6,7-difluoro-2-methyl-4-oxoquinazolin-3(4H)-yl)phenyl)-2-phenylacetamide (FS-31):To a stirred solution of 2-phenylacetic acid (5) (100 mg, 0.735 mmol, 1 equiv) and 3-(4-aminophenyl)-6,7-difluoro-2-methylquinazolin-4(3H)-one (4) (253 mg, 0.882 mmol 1.2 equiv) in DMF (1 mL), followed by DIPEA (0.4 mL, 2.205 mmol) and HATU (419 mg, 1.102 mmol, 1.5 equiv) was added at 0° C. The reaction mixture was then allowed to room temperature and stirred for 4 h. The reaction progress was monitored by TLC, after completion of reaction, the reaction mixture was diluted with cold water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-31 (42 mg, Yield-29%) as a brown solid.

LCMS: m/z=406.31 [M+H]⁺, 96.89% (2.73 min).

¹H NMR (400 MHz, DMSO-d₆) δ ppm: 5=10.43 (s, 1H), 8.02 (t, J=9.5 Hz, 1H), 7.74-7.79 (m, 3H), 7.31-7.38 (m, 6H), 7.24-7.30 (m, 1H), 3.69 (s, 2H), 2.13 (s, 3H).

### FS-32:

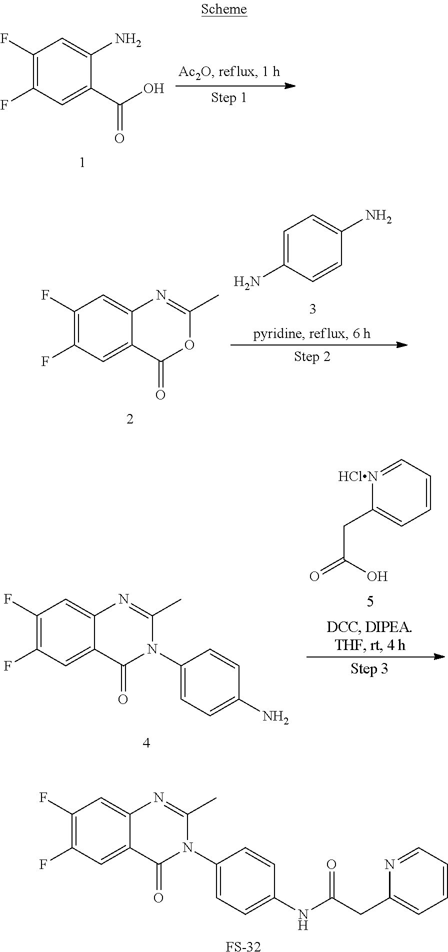

### Step-3:

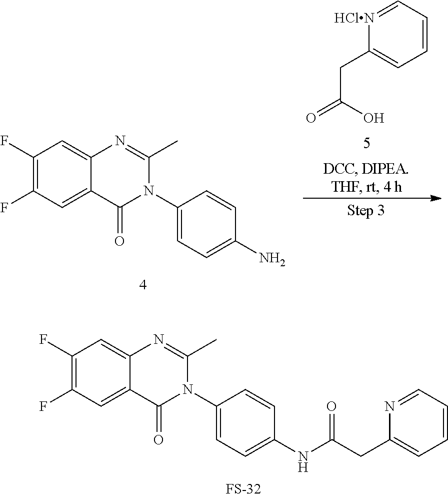

Synthesis of N-(4-(6,7-difluoro-2-methyl-4-oxoquinazolin-3(4H)-yl)phenyl)-2-(pyridin-2-yl)acetamide (FS-32): To a stirred solution of 2-(pyridin-2-yl)acetic acid, hydrochloride (5) (200 mg, 1.156 mmol, 1.0 equiv) and 3-(4-aminophenyl)-6,7-difluoro-2-methylquinazolin-4(3H)-one (4) (165 mg, 0.578 mmol, 0.5 equiv) in THF (2 mL) was added DIPEA (0.72 mL, 4.624 mmol, 4.0 eq) and DCC (595 mg, 2.890 mmol, 2.5 equiv) at 0° C. The reaction mixture was then allowed to room temperature and stirred for 3 h. The reaction was monitored by TLC, after completion of reaction, the reaction mixture was diluted with water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by reverse phase C18 column using 0.01% NH₃ buffer in acetonitrile/water and after lyophilisation, to give FS-32 (52 mg, Yield-37%) as an off white solid.

LCMS: m/z=407.29 [M+H]⁺, 95.46% (1.94 min).

¹H NMR (400 MHz, DMSO-d₆) δ ppm: 10.51 (br, s, 1H), 8.52 (d, J=4.0 Hz, 1H), 8.03 (t, J=9.6 Hz, 1H), 7.744-7.79 (m, 4H), 7.27-7.43 (m, 4H), 3.89 (s, 2H), 2.13 (br, s, 3H).

### FS-33:

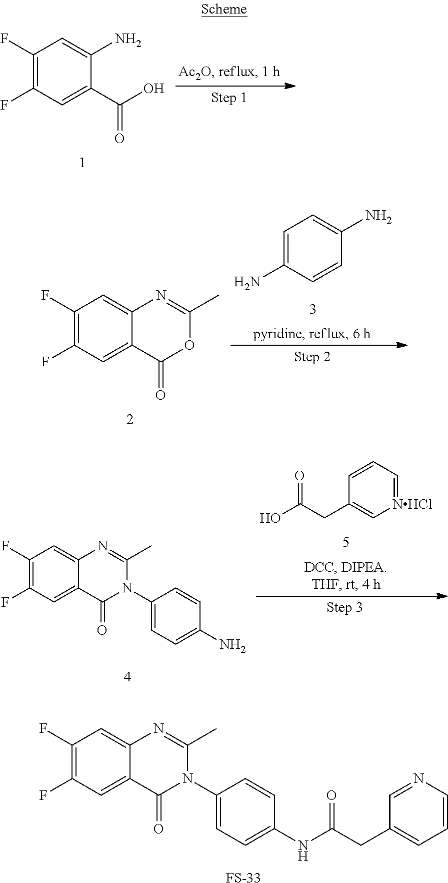

### Step-3:

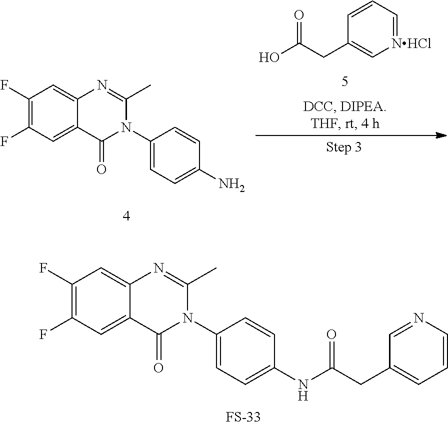

Synthesis of N-(4-(6,7-difluoro-2-methyl-4-oxoquinazolin-3(4H)-yl)phenyl)-2-(pyridin-3-yI)acetamide (FS-33): To a stirred solution of 3-(4-aminophenyl)-6,7-difluoro-2-methylquinazolin-4(3H)-one (4) (100 mg, 0.898 mmol, 1.0 equiv) and 2-(pyridin-3-yl)acetic acid hydrochloride (5) (150 mg, 0.898 mmol, 2.5 equiv) in THF (1 mL) was added DIPEA (0.2 mL, 0.522 mmol) and DCC (297 mg, 1.44 mmol, 2.5 equiv) at 0° C. The reaction mixture was then allowed to stir at room temperature for 3 h. The reaction was monitored by TLC, after completion of reaction, the reaction mixture was diluted with water (20 mL) and extracted with ethyl acetate (2×25 mL), separated the organic layer, washed with brine solution (20 mL). The combined organic layer was dried over anhydrous Na₂SO₄, filtered and concentrated under reduced pressure. The crude residue was purified by reverse phase C18 column using 0.01% NH₃ buffer in acetonitrile/water and after lyophilisation, to give FS-33 (52 mg, Yield-37%) as a brown solid.

LCMS: m/z=407.29 [M+H]⁺, 96.24% (1.90 min).

¹H NMR (400 MHz, DMSO-d₆) δ ppm: 10.49 (br, s, 1H), 8.54 (s, 1H), 8.47 (d, J=4.4 Hz, 1H), 8.02 (t, J=9.6 Hz, 1H), 7.75 (d, J=8.8 Hz, 4H), 7.37 (d, J=8.0 Hz, 3H), 3.75 (s, 2H), 2.12 (br, s, 3H).

### FS-34:

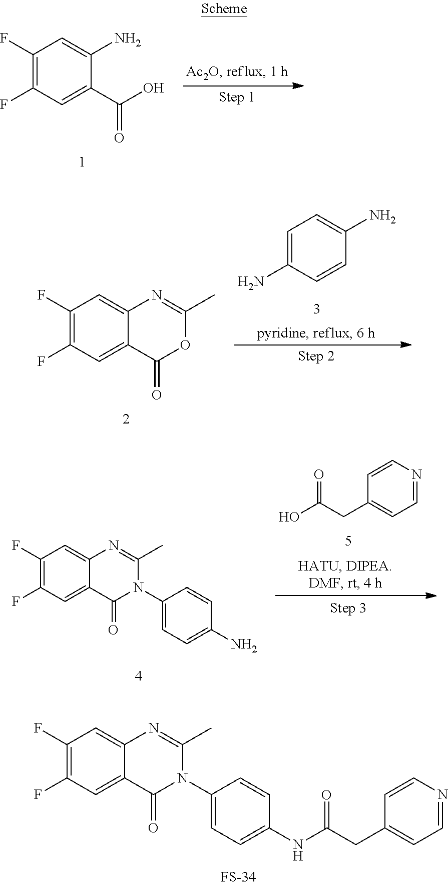

### Step-3:

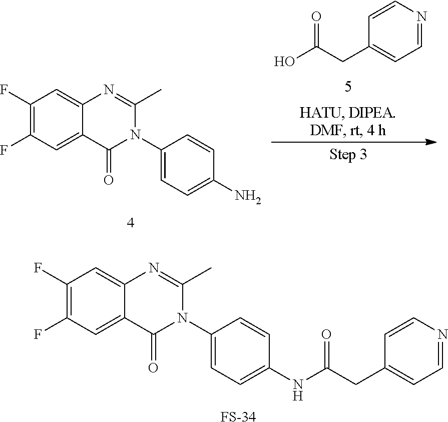 [SMILES: O=C(O)Cc1ccncc1 ; Cc1nc2cc(F)c(F)cc2c(=O)n1-c1ccc(N)cc1 ; Cc1nc2cc(F)c(F)cc2c(=O)n1-c1ccc(NC(=O)Cc2ccncc2)cc1 ; "FS-34"]

Synthesis of N-(4-(6,7-difluoro-2-methyl-4-oxoquinazolin-3(4H)-yl)phenyl)-2-(pyridin-4-yl)acetamide (FS-34):To a stirred solution of 2-(pyridin-4-yl)acetic acid (5) (100 mg, 0.729 mmol, 1 equiv) and 3-(4-aminophenyl)-6,7-difluoro-2-methylquinazolin-4(3H)-one (4) (251 mg, 0.875 mmol, 1.2 equiv) in DMF (1 mL), followed by added DIPEA (0.4 mL, 2.189 mmol) and HATU (416 mg, 1.094 mmol, 1.5 equiv) at 0° C. The reaction mixture was then allowed to room temperature and stirred for 3 h. The reaction was monitored by TLC, after completion of reaction, the reaction mixture was diluted with cold water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-34 (60 mg, Yield-42%) as a brown solid.

LCMS: m/z=407.28 [M+H]⁺, 99.80% (1.66 min).

¹H NMR (400 MHz, DMSO-d₆) δ ppm: 10.53 (br, s, 1H), 8.61 (d, J=4.8 Hz, 2H), 8.02 (t, J=8.4 Hz, 1H), 7.746-7.787 (m, 3H), 7.52 (d, J=6.0 Hz, 2H), 7.38 (d, J=8.8 Hz, 2H), 3.84 (br,s, 2H), 2.12 (br, s, 3H).

### FS-35:

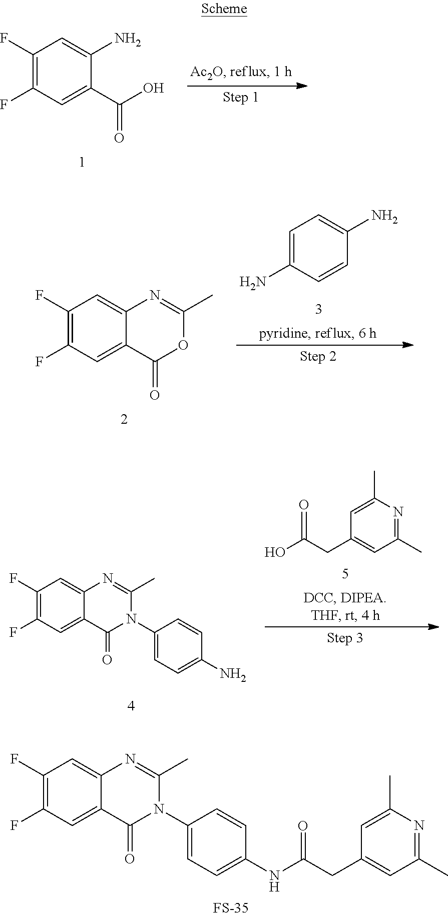

### Step-3:

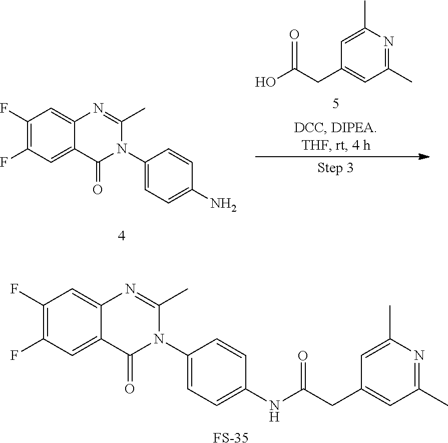

Synthesis of N-(4-(6,7-difluoro-2-methyl-4-oxoquinazolin-3(4H)-yl)phenyl)-2-(2,6-dimethylpyridin-4-yl)acetamide (FS-35):To a stirred solution of 2-(2,6-dimethylpyridin-4-yl)acetic acid (5) (50 mg, 0.248 mmol, 1 equiv) and 3-(4-aminophenyl)-6,7-difluoro-2-methylquinazolin-4(3H)-one (4) (71 mg, 0.248 mmol, 1.0 equiv) in THF (1 mL), followed by added DIPEA (0.4 mL, 2.189 mmol) and HATU (416 mg, 1.094 mmol, 1.5 equiv) at 0° C. The reaction mixture was then allowed to room temperature and stirred for 3 h. The reaction was monitored by TLC, after completion of reaction, the reaction mixture was diluted with cold water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by prep HPLC and after lyophilisation, to give FS-35. (8 mg, Yield-10%) as an off white solid.

LCMS: m/z=435.33 [M+H]⁺, 96.07% (1.58 min).

¹H NMR (400 MHz, DMSO-d₆) δ ppm: 10.46 (br, s, 1H), 8.02 (t, J=10 Hz, 1H), 7.77 (t, J=8.0 Hz, 3H), 7.37 (d, J=8.8 Hz, 2H), 7.01 (br, s, 2H), 3.64 (s, 2H), 2.41 (br, s, 6H), 2.07 (br, s, 3H).

### FS-37:

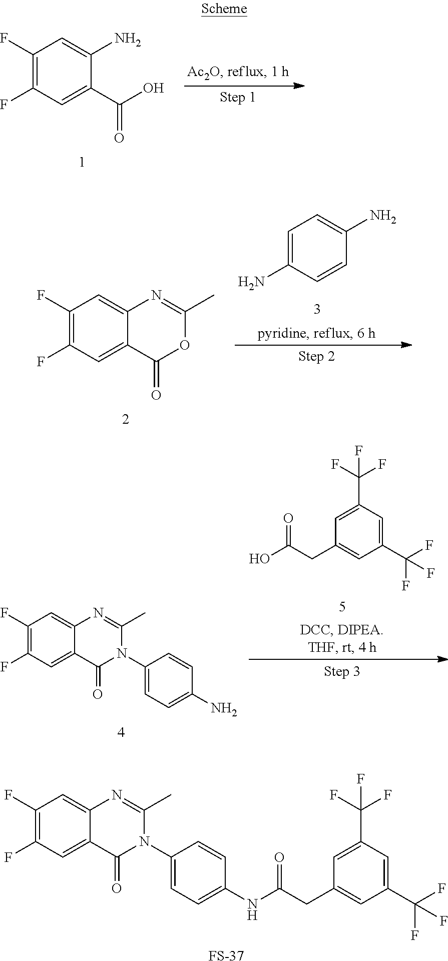

### Step-3:

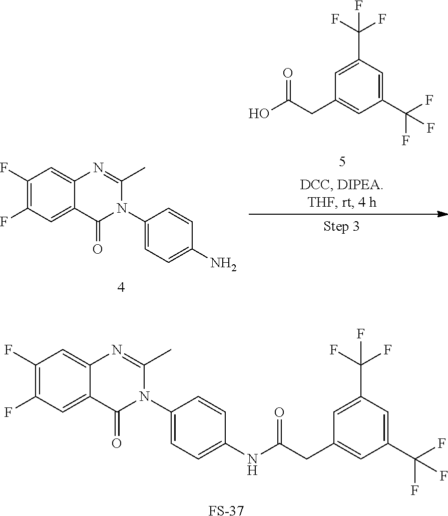 [SMILES: O=C(O)Cc1cc(C(F)(F)F)cc(C(F)(F)F)c1 ; Cc1nc2cc(F)c(F)cc2c(=O)n1-c1ccc(N)cc1 ; Cc1nc2cc(F)c(F)cc2c(=O)n1-c1ccc(NC(=O)Cc2cc(C(F)(F)F)cc(C(F)(F)F)c2)cc1 ; "FS-37"]

Synthesis of 2-(3,5-bis(trifluoromethyl)phenyl)-N-(4-(6,7-difluoro-2-methyl-4-oxoquinazolin-3(4H)-yl)phenyl)acetamide (FS-37):To a stirred solution of 2-(3,5-bis(trifluoromethyl)phenyl) acetic acid (5) (100 mg, 0.367 mmol, 1 equiv) and 3-(4-aminophenyl)-6,7-difluoro-2-methylquinazolin-4(3H)-one (4) (105 mg, 0.365 mmol, 1.0 equiv) in THF (1 mL), followed by added DIPEA (0.25 mL, 1.468 mmol) and DCC (99 mg, 0.917 mmol, 2.5 equiv) at 0° C. The reaction mixture was then allowed to room temperature and stirred for 3 h. The reaction was monitored by TLC, after completion of reaction, the reaction mixture was diluted with cold water (20 mL) and extracted with ethyl acetate (2×25 mL), separated the organic layer, washed with brine solution (20 mL). The combined organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-37. (48 mg, Yield-25%) as a white solid.

LCMS: m/z=542.29 [M+H]⁺, 95.01% (3.42 min).

¹H NMR (400 MHz, DMSO-d₆) δ ppm: 10.52 (br, s, 1H) 7.99-8.08 (m, 4H), 7.7 (d, J=4.0 Hz, 3H). 7.37 (d. J=8.8 Hz. 2H). 4.0 (br.s. 2H). 2.12 (br. s. 3H).

### FS-27:

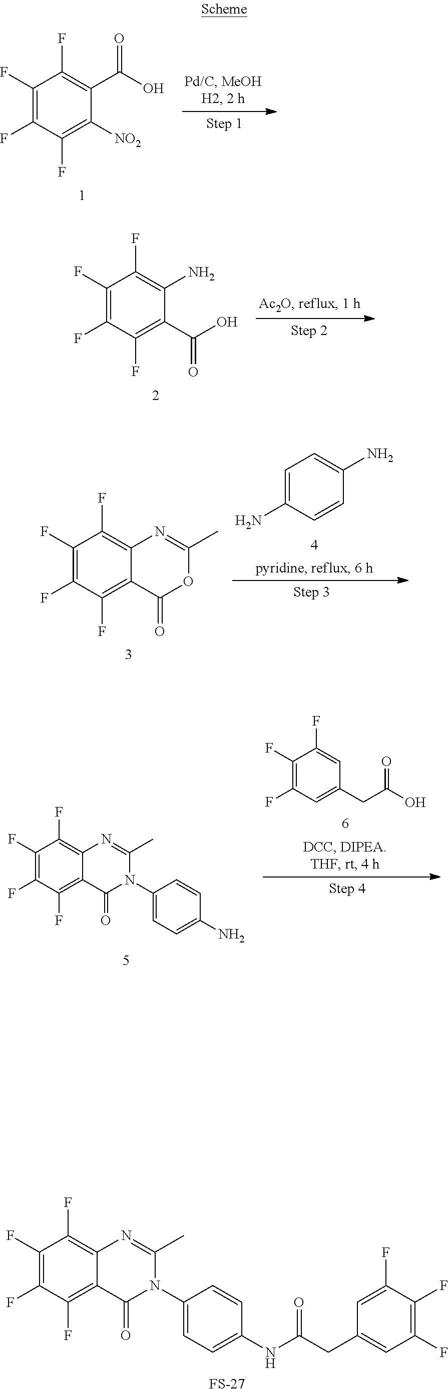

### Step-2:

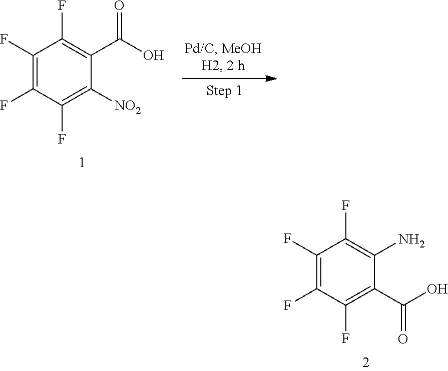 [SMILES: O=C(O)c1c(F)c(F)c(F)c(F)c1[N+](=O)[O-] ; Nc1c(F)c(F)c(F)c(F)c1C(=O)O]

Synthesis of 2-amino-3,4,5,6-tetrafluorobenzoic acid (2): A mixture of2,3,4,5-tetrafluoro-6-nitrobenzoic acid (1) (500 mg, 2.092 mmol, 1 equiv) in Methanol (5 mL) and 10% Pd/C (50 mg) was added slowly to reaction mixture under H₂ gas atmosphere. The reaction mixture was stirred at room temperature for 2 h. The reaction was monitored by TLC, after completion of reaction, the reaction mixture was filetered through the celite bed and washed with MeOH (2×20 mL). The filtrate was evaporated under reduced pressure to afford tittle compound-2. (420 mg, Yield- 96%) as a white solid.

LCMS: m/z =208.05 [M−H]⁺, 99.60% (1.22 min).

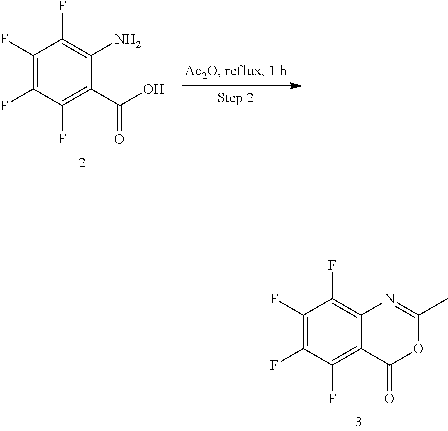 [SMILES: Nc1c(F)c(F)c(F)c(F)c1C(=O)O ; Cc1nc2c(F)c(F)c(F)c(F)c2c(=O)o1]

Synthesis of 5,6,7,8-tetrafluoro-2-methyl-4H-benzo[d][1,3]oxazin-4-one (3): A mixture of 2-amino-3,4,5,6-tetrafluorobenzoic acid (2) (400 mg, 1.913 mmol, 1 equiv) and acetic anhydride (2.5 mL) was stirred at room temperature for 15 minutes. The reaction mixture was then refluxed for 6 h. The reaction was monitored by TLC, after completion of reaction, the excess of acetic anhydride was removed under reduced pressure. The crude product was diluted with water (50 mL) and extracted with ethyl acetate (2×30 mL), separated the organic layer and washed with brine solution (20 mL). The organic layer was dried over anhydrous Na₂SO₄, filtered and concentrated under reduced pressure to 5,6,7,8-tetrafluoro-2-methyl-4H-benzo[d][1,3]oxazin-4-one (3) (352 mg, Yield-78%) as brown solid. The product was used for next step without further purification.

LCMS: m/z=206.08 [M−H]⁻, 89.22% (1.36 min).

### Step-3:

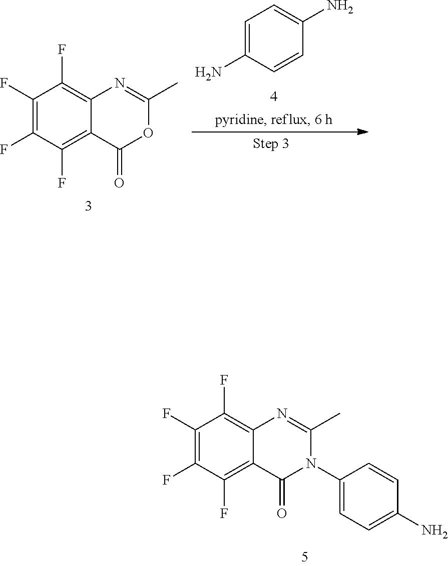

Synthesis of 3-(4-aminophenyl)-5,6,7,8-tetrafluoro-2-methylquinazolin-4(3H)-one (5): To a stirred solution of 5,6,7,8-tetrafluoro-2-methyl-4H-benzo[d][1,3]oxazin-4-one (3) (350 mg, 1.502 mmol, 1 equiv) and benzene-1,4-diamine (4) (162 mg, 1.502 mmol, 1 equiv) in 10 mL of anhydrous pyridine. The reaction mixture was refluxed for 6 h. The resulting mixture was cooled in ice bath and treated with 10 mL of 1N HCl to form a brown precipitated solid, washed with water and dried over air. The crude compound was recrystallized from ethanol to afford compound (5) (350 mg, 72% yield) as a brown solid. The product was directly used for next step without further purification.

LCMS: m/z=324.19 [M+H]⁺, 50.77% (1.32 min).

### Step-4:

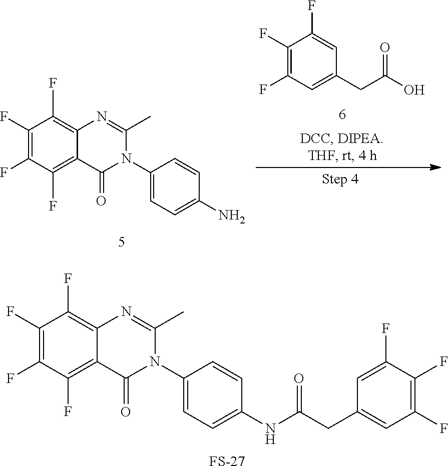

Synthesis of N-(4-(5,6,7,8-tetrafluoro-2-methyl-4-oxoquinazolin-3(4H)-yl)phenyl)-2-(3,4,5-trifluorophenyl)acetamide (FS-27): To a stirred solution of 2-(3,4,5-trifluorophenyl)acetic acid (6) (200 mg, 1.052 mmol, 1 equiv) and 3-(4-aminophenyl)-5,6,7,8-tetrafluoro-2-methylquinazolin-4(3H)-one (5) (339 mg, 1.052 mmol, 1 equiv) in THF (2 mL), DIPEA (0.5 mL, 4.208 mmol) and DCC (542 mg, 2.630 mmol, 2.5 equiv) was added at 0° C. The reaction mixture was then allowed to room temperature and stirred for 4 h. The reaction was monitored by TLC, after completion of reaction, the reaction mixture was diluted with cold water (20 mL) and extracted with ethyl acetate (2×25 mL), separated the organic layer, washed with brine solution (20 mL). The combined organic layer was dried over anhydrous Na₂SO₄, filtered and concentrated under reduced pressure. The crude residue was purified by prep HPLC and after lyophilisation, to give FS-27 (22 mg, Yield-12%) as an off white solid.

LCMS: m/z=496.23 [M+H]⁺, 98.84% (3.06 min).

¹H NMR (400 MHz, DMSO-d₆) δ ppm: 10.45 (s, 1H), 7.76 (d, J=8.0 Hz, 2H), 7.29-7.38 (m, 4H), 3.7 (br, s, 2H), 2.15 (br,s, 3H).

### FS-30:

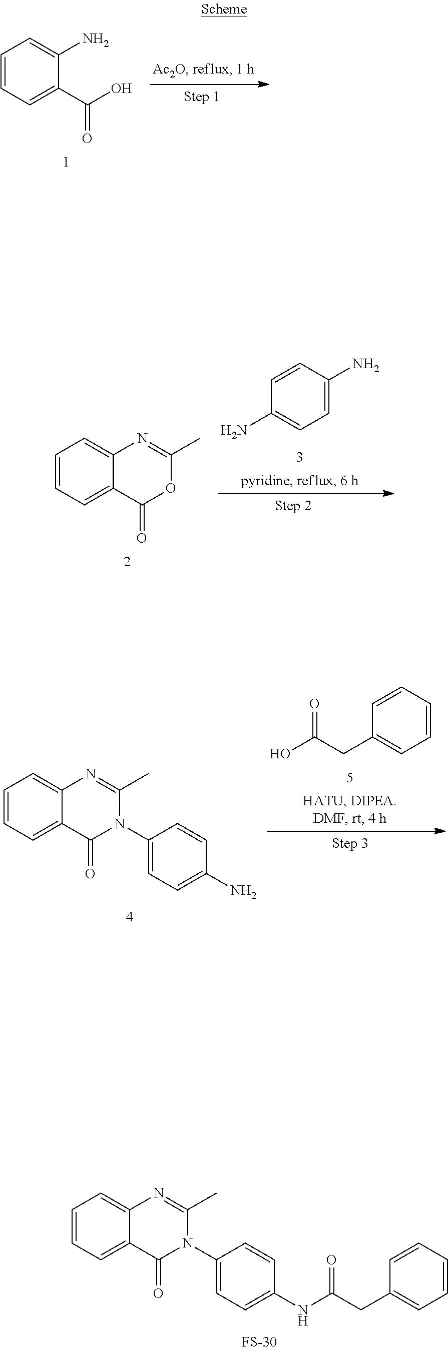

### Step-1:

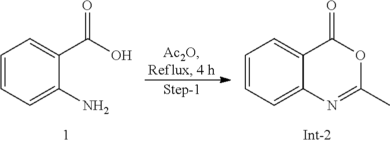

Synthesis of 2-methyl-4H-benzo[d][1,3]oxazin-4-one (Int-2): A mixture of 2-aminobenzoic acid (1) (1.0 g, 0.007 mmol, 1 equiv) and acetic anhydride (6 mL) was refluxed for 4 h. The reaction mixture was cooled to room temperature and the excess of acetic anhydride was removed under reduced pressure. The crude reaction mixture was diluted with water (20 mL) and extracted with ethyl acetate (2×50 mL), separated the organic layer and washed with brine solution (30 mL). The combined organic layer was dried over anhydrous Na₂SO₄, filtered and concentrated under reduced pressure to afford Int-2 (620 mg yield-44%) as an off-white solid. The product was directly used for next step without further purification.

LCMS: m/z=162.04 [M+H]⁺, 95.75% (1.154 min).

### Step-2:

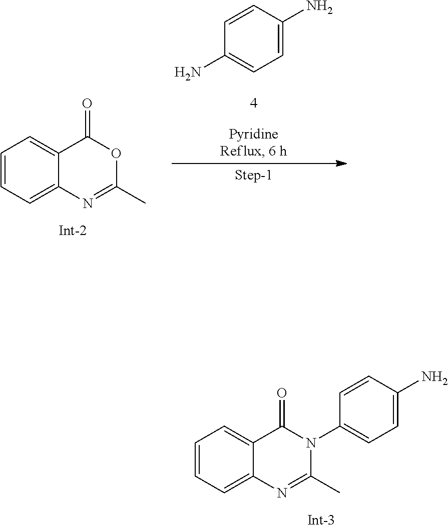 [SMILES: Nc1ccc(N)cc1 ; Cc1nc2ccccc2c(=O)o1 ; Cc1nc2ccccc2c(=O)n1-c1ccc(N)cc1]

Synthesis of 3-(4-aminophenyl)-2-methylquinazolin-4(3H)-one (Int-3): To a stirred solution of 2-methyl-4H-benzo[d][1,3]oxazin-4-one (2) (600 mg, 3.726 mmol, 1 equiv) and p-phenylenediamine (4) (402 mg, 3.726 mmol, 1.0 equiv) in 18 mL of anhydrous pyridine. The reaction mixture was refluxed for 6 h. The resulting solution was cooled in an ice bath and acidified with 10 mL of dilute hydrochloric acid to form a white precipitated solid and filtered, washed with water, dried. The crude residue was recrystallized from ethanol to afford Int-3 (720 mg, 67% yield) as brown solid.

LCMS: m/z=252.57 [M+H]⁺, 97.01% (1.04 min).

¹H NMR (400 MHz, DMSO-d₆) δ ppm: 8.16 (d, J=7.6 Hz, 1H), 7.97-7.89 (m, 2H), 7.64 (t, J=7.2 Hz, 1H), 7.50-7.39 (m, 4H), 2.32 (s, 3H).

### Step-3:

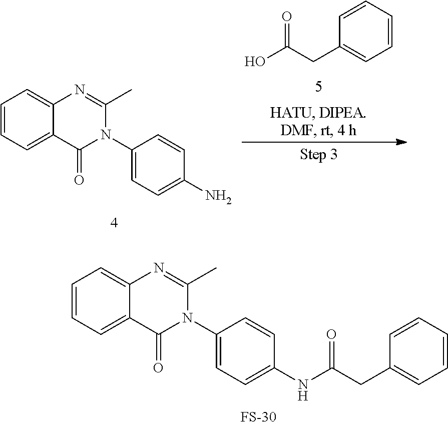

Synthesis of N-(4-(2-methyl-4-oxoquinazolin-3(4H)-yl)phenyl)-2-phenylacetamide (FS-30):To a stirred solution of 2-phenylacetic acid (5) (100 mg, 0.735 mmol, 1 equiv) and (4-aminophenyl)-2-methylquinazolin-4(3H)-one (4) (185 mg, 0.735 mmol 1.0 equiv) in DMF (1 mL) was added DIPEA (0.4 mL, 2.205 mmol) followed by HATU (419 mg, 1.102 mmol, 1.5 equiv) at 0° C. The reaction mixture was then allowed to stir at room temperature for 4 h. The reaction was monitored by TLC, after completion of reaction, the reaction mixture was diluted with cold water (20 mL) and extracted with ethyl acetate (2×25 mL), separated the organic layer, washed with brine solution (20 mL). The organic layer was dried over anhydrous Na₂SO₄, filtered and concentrated under reduced pressure. The crude residue was purified by reverse phase C18 column using 0.01% HCOOH buffer in acetonitrile/water and after lyophilisation, to give FS-30 (38 mg, Yield-27%) as a brown solid.

LCMS: m/z=370.25 [M+H]⁺, 99.03% (2.41 min).

¹H NMR (400 MHz, DMSO-d₆) δ ppm: 5=10.42 (s, 1H), 8.12 (d, J=12 Hz, 1H), 7.835-7.85 (m, 1H), 7.76 (d, J=8.8 Hz, 2H), 7.65 (d, J=14 Hz, 1H), 7.51 (t, J=7.2 Hz, 1H), 7.284-7.366 (m, 6H), 7.241-7.7.270 (m, 1H), 3.69 (br, s, 2H), 2.13 (s, 3H).

### 2. Biological Testing

### 2.1 Methods:

To identify new small molecule inhibitors that induce ferroptosis in FSP1 overexpressing cells lacking GPX4, hFSP1 GPX4 KO as well as FSP1 GPX4 wild type (WT) isogenic fibroblasts were screened using a subtractive high throughput screening (HTS) approach. As such, both cell lines were plated in parallel and screened against HMGU libraries at a fixed concentration of 10 μM. Cell survival was evaluated using the LIVE/DEAD assay Aquabluer. The percentage (%) of viable cells was determined for both wild type (WT) and knockout (KO) cell lines. Hits were considered as compounds that induce ferroptosis in KO but not in WT cells. Subsequently, positive hits were assayed in a dose-response manner to establish EC₅₀ values of respective compounds (see Table 2).

TABLE 2
Activity of tested compounds: * below 100 nM,
** below 1000 nM, *** above 1000 nM

| Compound | | hFSP1: |
|---|---|---|
| Number | Structure | EC50 [nM] |
| 1 | 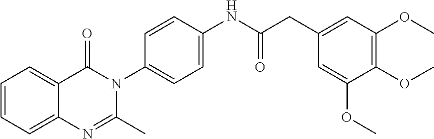 [SMILES: COc1cc(CC(=O)Nc2ccc(-n3c(C)nc4ccccc4c3=O)cc2)cc(OC)c1OC] | ** |
| 2 | 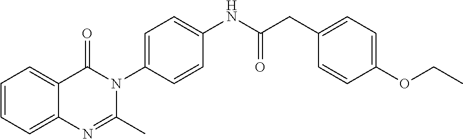 [SMILES: CCOc1ccc(CC(=O)Nc2ccc(-n3c(C)nc4ccccc4c3=O)cc2)cc1] | *** |
| 3 | 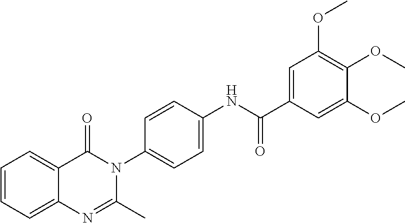 [SMILES: COc1cc(C(=O)Nc2ccc(-n3c(C)nc4ccccc4c3=O)cc2)cc(OC)c1OC] | *** |
| 4 | 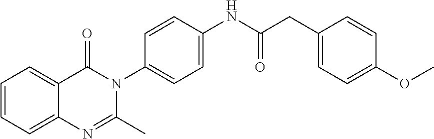 [SMILES: COc1ccc(CC(=O)Nc2ccc(-n3c(C)nc4ccccc4c3=O)cc2)cc1] | *** |
| 5 | 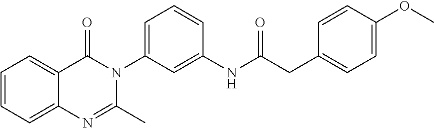 [SMILES: COc1ccc(CC(=O)Nc2cccc(-n3c(C)nc4ccccc4c3=O)c2)cc1] | *** |
| 6 | 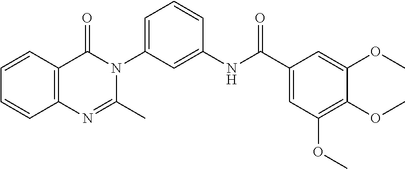 [SMILES: COc1cc(C(=O)Nc2cccc(-n3c(C)nc4ccccc4c3=O)c2)cc(OC)c1OC] | ** |
| 7 | 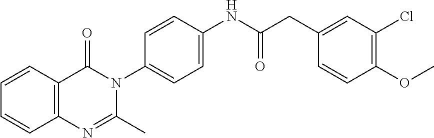 [SMILES: COc1ccc(CC(=O)Nc2ccc(-n3c(C)nc4ccccc4c3=O)cc2)cc1Cl] | ** |
| 8 | 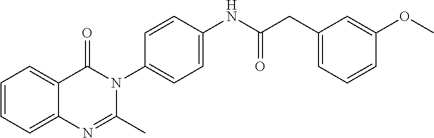 [SMILES: COc1cccc(CC(=O)Nc2ccc(-n3c(C)nc4ccccc4c3=O)cc2)c1] | *** |
| 9 | 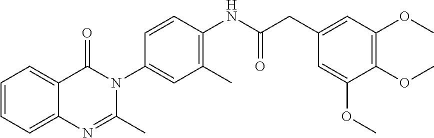 [SMILES: COc1cc(CC(=O)Nc2ccc(-n3c(C)nc4ccccc4c3=O)cc2C)cc(OC)c1OC] | ** |
| 10 | 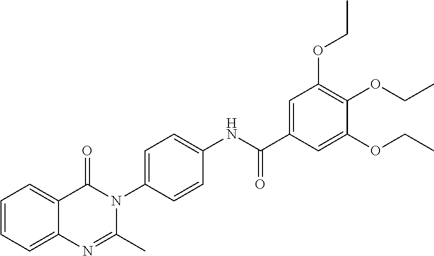 [SMILES: CCOc1cc(C(=O)Nc2ccc(-n3c(C)nc4ccccc4c3=O)cc2)cc(OCC)c1OCC] | ** |
| 11 | 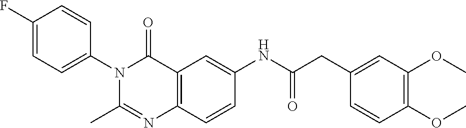 [SMILES: COc1ccc(CC(=O)Nc2ccc3nc(C)n(-c4ccc(F)cc4)c(=O)c3c2)cc1OC] | *** |
| 12 | 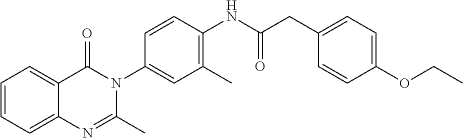 [SMILES: CCOc1ccc(CC(=O)Nc2ccc(-n3c(C)nc4ccccc4c3=O)cc2C)cc1] | ** |
| 13 | 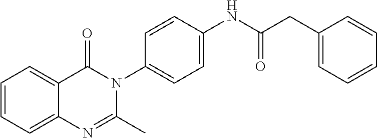 [SMILES: Cc1nc2ccccc2c(=O)n1-c1ccc(NC(=O)Cc2ccccc2)cc1] | *** |
| 14 | 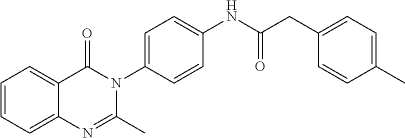 [SMILES: Cc1ccc(CC(=O)Nc2ccc(-n3c(C)nc4ccccc4c3=O)cc2)cc1] | ** |
| 15 | 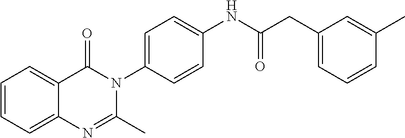 [SMILES: Cc1cccc(CC(=O)Nc2ccc(-n3c(C)nc4ccccc4c3=O)cc2)c1] | ** |
| 16 | 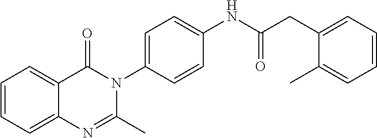 [SMILES: Cc1ccccc1CC(=O)Nc1ccc(-n2c(C)nc3ccccc3c2=O)cc1] | *** |
| 17 | 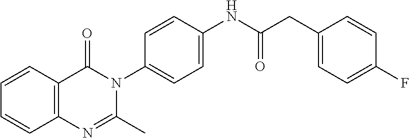 [SMILES: Cc1nc2ccccc2c(=O)n1-c1ccc(NC(=O)Cc2ccc(F)cc2)cc1] | *** |
| 18 | 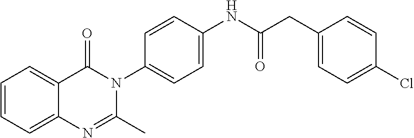 [SMILES: Cc1nc2ccccc2c(=O)n1-c1ccc(NC(=O)Cc2ccc(Cl)cc2)cc1] | ** |
| 19 | 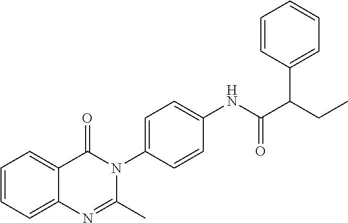 [SMILES: CCC(C(=O)Nc1ccc(-n2c(C)nc3ccccc3c2=O)cc1)c1ccccc1] | ** |
| 20 | 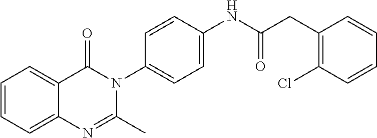 [SMILES: Cc1nc2ccccc2c(=O)n1-c1ccc(NC(=O)Cc2ccccc2Cl)cc1] | *** |
| 21 | 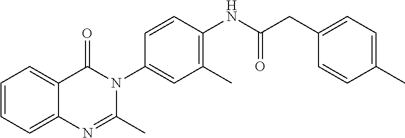 [SMILES: Cc1ccc(CC(=O)Nc2ccc(-n3c(C)nc4ccccc4c3=O)cc2C)cc1] | *** |
| 22 | 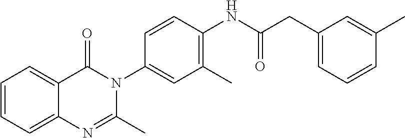 [SMILES: Cc1cccc(CC(=O)Nc2ccc(-n3c(C)nc4ccccc4c3=O)cc2C)c1] | *** |
| 23 | 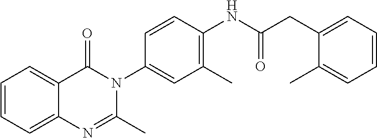 [SMILES: Cc1ccccc1CC(=O)Nc1ccc(-n2c(C)nc3ccccc3c2=O)cc1C] | *** |
| 24 | 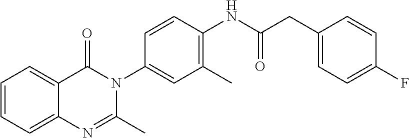 [SMILES: Cc1cc(-n2c(C)nc3ccccc3c2=O)ccc1NC(=O)Cc1ccc(F)cc1] | *** |
| 25 | 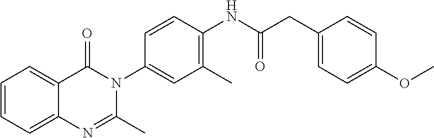 [SMILES: COc1ccc(CC(=O)Nc2ccc(-n3c(C)nc4ccccc4c3=O)cc2C)cc1] | *** |
| 26 | 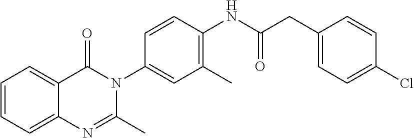 [SMILES: Cc1cc(-n2c(C)nc3ccccc3c2=O)ccc1NC(=O)Cc1ccc(Cl)cc1] | ** |
| 27 | 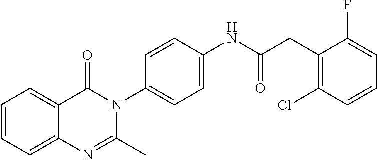 [SMILES: Cc1nc2ccccc2c(=O)n1-c1ccc(NC(=O)Cc2c(F)cccc2Cl)cc1] | ** |
| 28 | 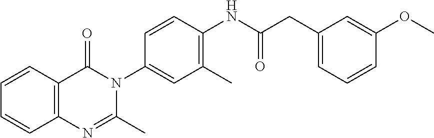 [SMILES: COc1cccc(CC(=O)Nc2ccc(-n3c(C)nc4ccccc4c3=O)cc2C)c1] | *** |
| 29 | 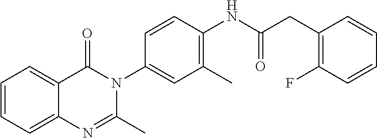 [SMILES: Cc1cc(-n2c(C)nc3ccccc3c2=O)ccc1NC(=O)Cc1ccccc1F] | *** |
| 30 | 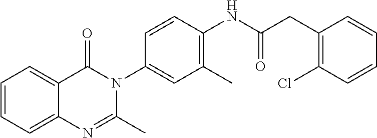 [SMILES: Cc1cc(-n2c(C)nc3ccccc3c2=O)ccc1NC(=O)Cc1ccccc1Cl] | *** |
| 31 | 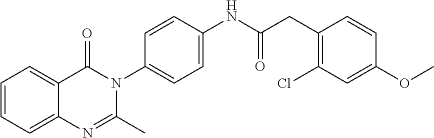 [SMILES: COc1ccc(CC(=O)Nc2ccc(-n3c(C)nc4ccccc4c3=O)cc2)c(Cl)c1] | ** |
| 32 | 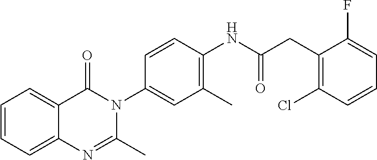 [SMILES: Cc1cc(-n2c(C)nc3ccccc3c2=O)ccc1NC(=O)Cc1c(F)cccc1Cl] | ** |
| 33 | 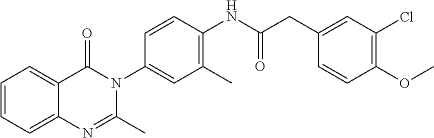 [SMILES: COc1ccc(CC(=O)Nc2ccc(-n3c(C)nc4ccccc4c3=O)cc2C)cc1Cl] | ** |
| 34 | 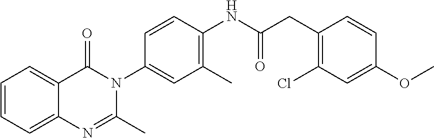 [SMILES: COc1ccc(CC(=O)Nc2ccc(-n3c(C)nc4ccccc4c3=O)cc2C)c(Cl)c1] | ** |
| 40 | 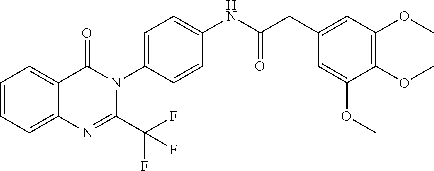 [SMILES: COc1cc(CC(=O)Nc2ccc(-n3c(C(F)(F)F)nc4ccccc4c3=O)cc2)cc(OC)c1OC] | ** |
| 41 | 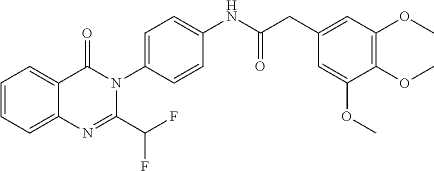 [SMILES: COc1cc(CC(=O)Nc2ccc(-n3c(C(F)F)nc4ccccc4c3=O)cc2)cc(OC)c1OC] | *** |
| 42 | 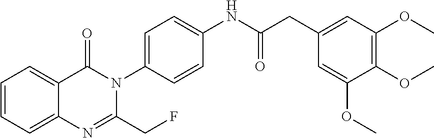 [SMILES: COc1cc(CC(=O)Nc2ccc(-n3c(CF)nc4ccccc4c3=O)cc2)cc(OC)c1OC] | ** |
| 46 | 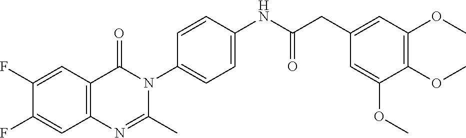 [SMILES: COc1cc(CC(=O)Nc2ccc(-n3c(C)nc4cc(F)c(F)cc4c3=O)cc2)cc(OC)c1OC] | * |
| 49 | 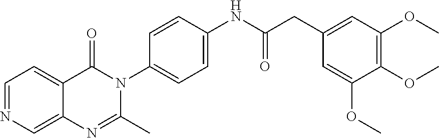 [SMILES: COc1cc(CC(=O)Nc2ccc(-n3c(C)nc4cnccc4c3=O)cc2)cc(OC)c1OC] | *** |
| 50 | 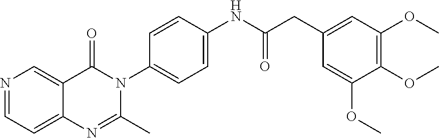 [SMILES: COc1cc(CC(=O)Nc2ccc(-n3c(C)nc4ccncc4c3=O)cc2)cc(OC)c1OC] | *** |
| 63 | 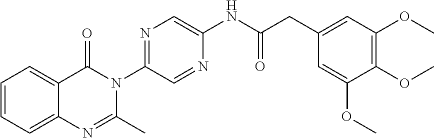 [SMILES: COc1cc(CC(=O)Nc2cnc(-n3c(C)nc4ccccc4c3=O)cn2)cc(OC)c1OC] | *** |
| 64 | 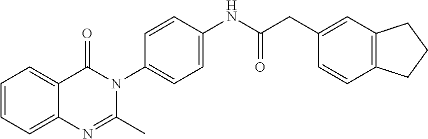 [SMILES: Cc1nc2ccccc2c(=O)n1-c1ccc(NC(=O)Cc2ccc3c(c2)CCC3)cc1] | * |
| 68 | 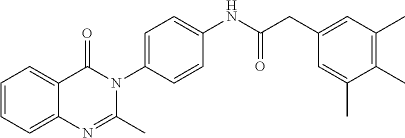 [SMILES: Cc1cc(CC(=O)Nc2ccc(-n3c(C)nc4ccccc4c3=O)cc2)cc(C)c1C] | * |
| 69 | 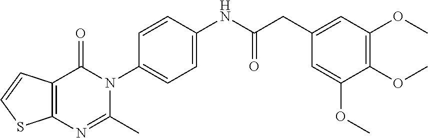 [SMILES: COc1cc(CC(=O)Nc2ccc(-n3c(C)nc4sccc4c3=O)cc2)cc(OC)c1OC] | ** |
| 71 | 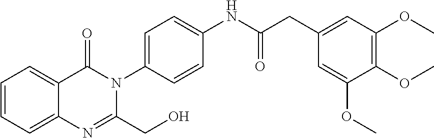 [SMILES: COc1cc(CC(=O)Nc2ccc(-n3c(CO)nc4ccccc4c3=O)cc2)cc(OC)c1OC] | *** |
| 72 | 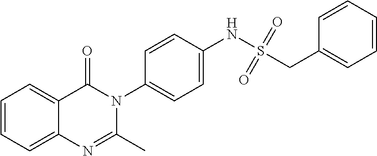 [SMILES: Cc1nc2ccccc2c(=O)n1-c1ccc(NS(=O)(=O)Cc2ccccc2)cc1] | *** |
| 73 | 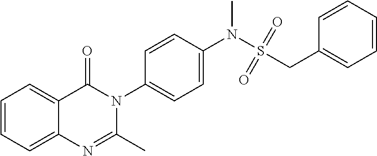 [SMILES: Cc1nc2ccccc2c(=O)n1-c1ccc(N(C)S(=O)(=O)Cc2ccccc2)cc1] | *** |
| 74 | 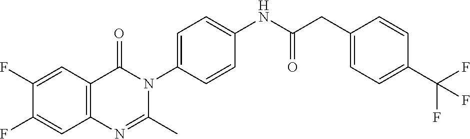 [SMILES: Cc1nc2cc(F)c(F)cc2c(=O)n1-c1ccc(NC(=O)Cc2ccc(C(F)(F)F)cc2)cc1] | ** |
| 75 | 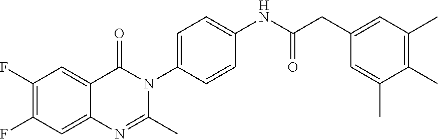 [SMILES: Cc1cc(CC(=O)Nc2ccc(-n3c(C)nc4cc(F)c(F)cc4c3=O)cc2)cc(C)c1C] | * |
| 76 | 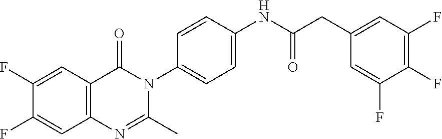 [SMILES: Cc1nc2cc(F)c(F)cc2c(=O)n1-c1ccc(NC(=O)Cc2cc(F)c(F)c(F)c2)cc1] | ** |
| 77 | 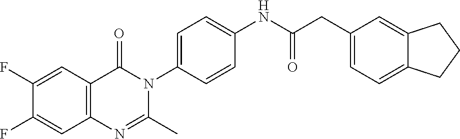 [SMILES: Cc1nc2cc(F)c(F)cc2c(=O)n1-c1ccc(NC(=O)Cc2ccc3c(c2)CCC3)cc1] | * |
| 78 | 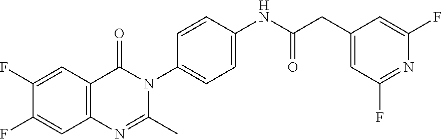 [SMILES: Cc1nc2cc(F)c(F)cc2c(=O)n1-c1ccc(NC(=O)Cc2cc(F)nc(F)c2)cc1] | ** |
| 79 | 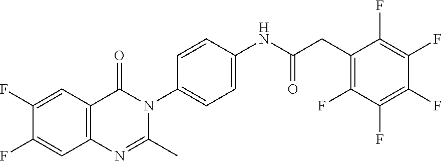 [SMILES: Cc1nc2cc(F)c(F)cc2c(=O)n1-c1ccc(NC(=O)Cc2c(F)c(F)c(F)c(F)c2F)cc1] | * |
| 80 | 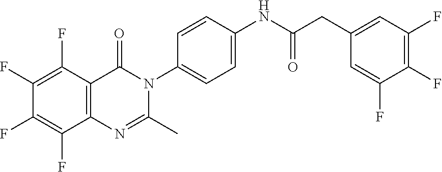 [SMILES: Cc1nc2c(F)c(F)c(F)c(F)c2c(=O)n1-c1ccc(NC(=O)Cc2cc(F)c(F)c(F)c2)cc1] | ** |
| 81 | 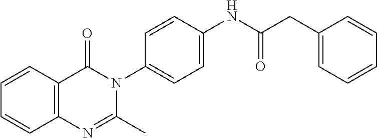 [SMILES: Cc1nc2ccccc2c(=O)n1-c1ccc(NC(=O)Cc2ccccc2)cc1] | ** |
| 82 | 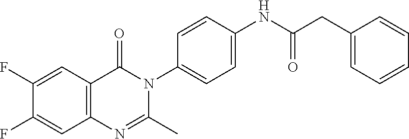 [SMILES: Cc1nc2cc(F)c(F)cc2c(=O)n1-c1ccc(NC(=O)Cc2ccccc2)cc1] | ** |
| 83 | 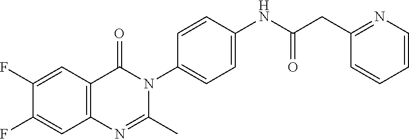 [SMILES: Cc1nc2cc(F)c(F)cc2c(=O)n1-c1ccc(NC(=O)Cc2ccccn2)cc1] | *** |
| 84 | 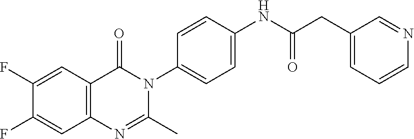 [SMILES: Cc1nc2cc(F)c(F)cc2c(=O)n1-c1ccc(NC(=O)Cc2cccnc2)cc1] | *** |
| 85 | 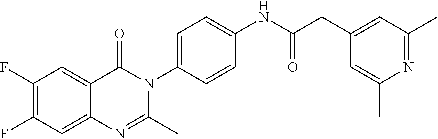 [SMILES: Cc1cc(CC(=O)Nc2ccc(-n3c(C)nc4cc(F)c(F)cc4c3=O)cc2)cc(C)n1] | *** |
| 86 | 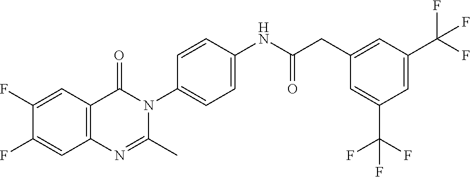 [SMILES: Cc1nc2cc(F)c(F)cc2c(=O)n1-c1ccc(NC(=O)Cc2cc(C(F)(F)F)cc(C(F)(F)F)c2)cc1] | * |

In addition, different concentrations of hit compounds were tested for their in vitro ability to inhibit the FSP1-mediated reduction of resazurin according to Mishima et al. (2022). Assays were carried out in TBS buffer (50 mM TRIS, 150 mM NaCl (pH8)), 100 nM resazurin, 200 μM NADH and 50 nM recombinant FSP1 enzyme. The fluorescence emission of reduced resazurin (Ex540/Em590) every 30 sec-60 sec using a SpectraMax or SpectraMaxiD5 μlate reader. Reaction speed was compared to DMSO containing control reactions. 2.2 Subcutaneous tumor model

All mice were obtained from Charles River. For the syngeneic subcutaneous tumor experiments, Gpx4^{KO}/Fsp1^{KO} 1B16F10 cells stably overexpressing hFSP1-HA (1×10⁶ cells in 100 μL PBS) were injected subcutaneously into the right flank of 7-week-old female C57BL6/J mice. After tumors reached a size of approximately 25-50 mm³, mice were randomized and treated with vehicle or icFSP1 (50 mg/kg, Intonation) by i.p. injections twice per day for 4-5 days. For generating tumor samples for staining, Gpx4^{KO}/Fsp1^{KO} 1B16F10 cells stably expressing hFSP1-WT-HA or hFSP1-Q319K-HA (1×10⁶ cells in 100 μL PBS) were injected subcutaneously into the right flank of 7-week-old female C57BL6/J mice. After tumors reached a size of approximately 50-150 mm³, mice were treated with vehicle or icFSP1 (50 mg/kg, Intonation) by i.p. injections twice per day for 3 days.

For the xenograft subcutaneous tumor experiments, GPX4^{KO} A375 cells (5×10⁶ cells in 100 μL PBS) were injected subcutaneously into the right flank of 7-week-old female athymic nude mice. After tumors reached a size of approximately 25-100 mm³, mice were randomized and treated with vehicle or icFSP1 (50 mg/kg, Intonation) by i.p. injections twice per day for the first 4 days and afterward once daily.

For the xenograft subcutaneous tumor experiments, GPX4^{KO} H460 cells (5×10⁶ cells in 100 μL PBS) were injected subcutaneously into the right flanks of 6-week-old female athymic nude mice. After tumors reached a size of approximately 100 mm³, mice were randomized and treated with vehicle or icFSP1 (50 mg/kg, Intonation) by i.p. injections twice per day.

icFSP1 was dissolved in 45% PEG E 300 (Sigma, Cat #91462-1 KG) and 55% PBS (Gibco, Cat #14190094). Tumors were measured by caliper every day and tumor volume was calculated by the following formula: Tumor volume=length×width²×0.52. When tumor sizes were either larger than 1000 mm³ at measurement or tumors became necrotic, these tumors were considered to be human endpoints. When tumors became human endpoints, the experiment was stopped at that point and no further experiments were conducted.

### 2.3 Cell Viability Assay

Cells were seeded on 96-well plates and cultured overnight. On the next day, the medium was changed to medium containing following compounds: RSL3, ML210, erastin, FIN56, FINO2, BSO, iFSP1, icFSP1, Lip-1, DFO, Fer-1, zVAD, Nec-1s, MCC950, olaparib, STS, TNFa, Smac mimic or nigericin at the indicated concentrations. For TAM and Dox treatment, cells were seeded with compounds at the same time. Cell viability was determined 1 h (for nigericin), 24-48 h (for RSL3, ML210, erastin, FIN56, FINO2, iFSP1, icFSP1, STS, TNFa, Smac mimic, and zVAD) or 72 h (for BSO, icFSP1, TAM, and Dox) after the treatment using AquaBluer (MultiTarget Pharmaceuticals, Cat #6015) as an indicator of viable cells according to the manufacturer's protocols. For apoptosis induction, HT-1080 cells were incubated with different concentrations of STS for 24 h. For necroptosis induction, HT-29 cells were incubated with different concentrations of TNFa with Smac mimic (400 nM), and zVAD (30 μM) for 24 h. For pyroptosis induction, LPS (1 μg/mL, 2 h)-stimulated THP-1 cells were incubated with nigericin for 1 h. For ferroptosis induction, cells were incubated with ferroptosis inducers for 24-72 h. As readout fluorescence was measured at Ex/Em=540/590 nm using a SpectraMax M5 microplate reader with SoftMax Pro v7 (Molecular devices) after 4 hours of incubation in normal cell culture conditions. The relative cell viability (%) was calculated as follows: (fluorescence (FL) of samples−background)/(FL of appropriate control samples−background)×100.

### 2.4 LDH Release Assay

Cells were seeded on 96-well plates and cultured overnight. On the next day, medium was changed to medium containing compounds and incubated for another 24 h. Cell death rates were measured using the Cytotoxicity Detection kit (LDH) (Roche, Cat #11644793001) in principle following the manufacturer's protocol. In brief, cell culture supernatant was collected as media sample. Cells were then lysed with PBS containing 0.1% Triton X-100 as lysate sample. Media and lysate samples were individually mixed with reagents on microplates, and the absorbance was measured at 492 nm using SpectraMax M5 microplate reader after 15-30 min incubation at room temperature. Cell death ratio was calculated by LDH release (%) as follows: [absorbance (abs) of medium samples−background]/[(abs of lysate samples−background)+(abs of medium samples−background)]×100.

### 2.5 Lipid Peroxidation Assay

100,000 cells per well were seeded on a 12-well plate one day prior to the experiments. On the next day, cells were treated with 2.5 μM icFSP1 for 3 h, and then incubated with 1.5 μM C11-BODIPY 581/591 (Invitrogen, Cat #D³⁸⁶¹) for 30 min in a 5% CO₂ atmosphere at 37° C. Subsequently, cells were washed by PBS once and trypsinized, and then resuspended in 500 μL PBS. Cells were passed through a 40 μm cell strainer and analyzed by a flow cytometer (CytoFLEX, Beckman Coulter) with a 488-nm laser for excitation. Data was collected from the FITC detector (for the oxidized form of BODIPY) with a 525/40 nm bandpass filter and from the PE detector (for the reduced form of BODIPY) with a 585/42 nm bandpass filter using CytExpert v2.4 (Beckman Coulter). At least 10,000 events were analyzed per sample. Data was analyzed using FlowJo Software (FlowJo LLC). Ratio of fluorescence of C11-BODIPY 581/591 (lipid peroxidation) [FITC/PE ratio (oxidized/reduced ratio)] was calculated as follows²²: (median of FITC-A fluorescence−median of FITC-A fluorescence of unstained samples)/(median of PE-A fluorescence−median of PE-A fluorescence of unstained samples).

### 2.6 Oxilipidomics Analysis

Two million cells were seeded on 15 cm dishes one day prior to the experiments. On the next day, cells were treated with 5 μM icFSP1 to induce lipid peroxidation. 5 hours later, cells were collected and sampled to liquid nitrogen and stored at ~80° C. Lipids from cells were extracted using the methyl-tert-butyl ether (MTBE) method. Briefly, cell pellets were collected in phosphate-buffered saline (PBS) containing dibutylhydroxytoluene (BHT, 100 μM) and diethylenetriamine pentaacetate (DTPA, 100 μM) were washed and centrifuged. SPLASH® LIPIDOMIX® (Avanti Polar Lipids Inc.) was added (2.5 μL) and incubated on ice for 15 min. After ice cold methanol (375 μL) and MTBE (1250 μL) were added, samples were vortexed and incubated for 1 h at 4° C. (Orbital shaker, 32 rpm). Phase separation was induced by addition of water (375 μL), vortexed, incubated for 10 min at 4° C. (Orbital shaker, 32 rpm), and centrifuged to separate organic and aqueous phase (10 min, 4° C., 1500×g). Organic phase was collected, dried in the vacuum evaporator and redissolved in 100 μL of isopropanol. Lipid extracts were transferred into glass vials for LC-MS analysis.

Reversed phase liquid chromatography (RPLC) was carried out on a Shimadzu ExionLC equipped with an Accucore C30 column (150×2.1 mm; 2.6 μm, 150 Å, Thermo Fisher Scientific). Lipids were separated by gradient elution with solvent A (acetonitrile/water, 1:1, v/v) and B (isopropanol/acetonitrile/water, 85:15:5, v/v) both containing 5 mM NH₄HCO₂ and 0.1% (v/v) formic acid. Separation was performed at 50° C. with a flow rate of 0.3 mL/min using following gradient: 0-20 min-10 to 86% B (curve 4), 20-22 min-86 to 95% B (curve 5), 22-26 min-95% isocratic, 26-26.1 min-95 to 10% B (curve 5) followed by 5 min re-equilibration at 10% B²³. Mass spectrometry analysis was performed on a Sciex 7500 system equipped with an electrospray (ESI) source and operated in negative ion mode. Products were analyzed in MRM mode monitoring transitions from the parent ion to daughter ion with the following parameters: TEM 500° C., GS1 40, GS2 70, CUR 40, CAD 9, IS—3000 V.

The area under the curve for the parent ion to daughter ion was integrated and normalized by appropriate lipid species, PC(15:0/18:1(d7)) or PE(15:0/18:1(d7)), from SPLASH LIPIDOMIX Mass Spec Standard (Avanti). Normalized peak areas were further log-transformed and auto-scaled in MetaboAnalyst online platform v5.0 (https://www.metaboanalyst.ca). Zero values were replaced by 0.2×the minimum values detected for a given oxidized lipid within the samples. Oxidized lipids showing a significant difference (ANOVA, adjusted P-value (false discovery rate (FDR)) cutoff: 0.05) between samples were used for the heat maps. The heat maps were created in GraphPad Prism 9. The color scheme corresponds to auto-scaled log fold change relative to the mean log value within the samples.

### 2.7 In Vitro Saturation Transfer Difference Experiments

Saturation transfer difference (STD) experiments were performed at Bruker Avance III HD spectrometer at 600 MHz ¹H frequency using a H/N/C triple resonance cryogenic probe. Spectra were recorded at 10° C. with 5 μM recombinant human FSP1 (mutant) and 100-fold molar excess of icFSP1 in phosphate-buffer saline with additional 150 mM NaCl, 1% (v/v) DMSO-d6 and 10% (v/v) D₂O for deuterium-lock. Saturation time was 2.5 sec and on- and off-frequencies were 0.68 and −17 ppm, respectively. NMR spectra were processed using Topspin 4.0.6 (Bruker).

### 2.8 Immunocytochemistry

All confocal microscopy images were acquired and analyzed using LSM880 microscope (ZEISS) with a 63× objective, and the corresponding appropriate filter sets to fluorophores and analyzed by ZEN Blue software (v3.2, ZWISS) or ImageJ/Fiji unless mentioned otherwise. Cells were seeded on p-Slide 8 well (ibidi, Cat #80826) one day prior to experiments. Next day, the medium was changed to fresh cell culture medium supplemented with 2.5 μM icFSP1. After incubation for the indicated times, cells were fixed and stained according to the following procedure: fixation with 4% paraformaldehyde for 5-10 min; permeabilization and blocking for 15 min with 0.3% Triton X-100 and 10 mg/mL BSA in PBS with DAPI (1:10,000) in TBS-T or PBS for 5 min at room temperature avoiding light. Finally, all samples were mounted in Aqua-/poly Mount (Polysciences, Cat #18606-20) and dried at 4° C. overnight.

2.9 Fluorescence Recovery after Photobleaching Assay (FRAP)

Pfa1 cells (20,000 cells) were seeded on p-Slide VI 0.4 (Ibidi, Cat #80606) one day prior to the experiments. On the next day, the medium was changed to DMEM-high glucose supplemented with 10% FBS, 2 mM L-glutamine, and 1% penicillin/streptomycin, 2.5 μM icFSP1 and 10 mM HEPES. After incubation with icFSP1 for 2-4 hours, 2-5 rectangular areas which contain more than three FSP1 condensates were selected as bleaching areas. Then, one image before bleaching areas was considered as time “0”. Subsequently, selected areas were photobleached by the maximum intensity of the laser, and FRAP was monitored at minimum intervals (~5 sec) using LSM880 microscope (ZEISS).

To quantify the FRAP rate, the region of interest (ROI) of each condensate (i) in the photobleached area and one condensate (c) in a non-photobleached area was determined by ImageJ/Fiji, then the mean value of fluorescence intensity of condensate i at time t, fᵢ(t) was obtained. After getting each time of fluorescence value, fᵢ(t) was normalized by the value of fᵢ(0) to get relative fluorescence (Rfᵢ(t)) of each bleached condensates. To reflect the quenching effects during observation and photobleaching, each Rfᵢ(t) was normalized by the corresponding time of relative value of non-bleached condensates (f_{c}(t)/f_{c}(0)) as follows: Fi(t)=Rfᵢ(t)/Rf_{c}(t)=[fᵢ(t)/fᵢ(0)]/[f_{c}(t)/f_{c}(0)]. Finally, the FRAP rate [%] at time t in the particles was calculated as the mean of Fᵢ(t)×100.

### 2.10 Live Cell Imaging

For co-staining or washout analyses, Pfa1 cells (15,000-30,000 cells) were seeded on p-Dish 35 mm low (Ibidi, Cat #80136), and incubated overnight. On the next day, cell culture medium was changed to FluoroBrite DMEM (Gibco, Cat #A1896701) supplemented with 10% FBS, 2 mM L-glutamine, and 1% penicillin/streptomycin. Live cell microscopy was performed using 3D Cell Explorer (Nanolive) using Eve v1.8.2 software with the corresponding appropriate filter sets. During imaging, the cells were maintained at 37° C. and 5% CO₂ atmosphere using a temperature-controlled incubation chamber. For co-staining analysis, cells were pre-treated for 1 h with 5 μM Liperfluo (Dojindo, Cat #L248-10), then changed to 0.2 μg/mL propidium iodide (PI, Sigma, P4170) containing FluoroBrite DMEM medium and started acquisition using Nanolive. After recording one image, a 100-fold concentration of icFSP1 in FluoroBrite DMEM was added to dishes (final concentration was 10 μM), while continuing recording images. Images were taken every 10 mins for more than 4 hours, and GFP, BFP and RFP filter sets were used for acquiring signals. For washout experiments, DMEM-high glucose medium was changed to FluoroBrite DMEM medium prior to the experiments followed by data acquisition using Nanolive. After recording a few images, a 100-fold concentration of icFSP1 in FluoroBrite DMEM was added to dishes (final concentration was 2.5 μM) and the recording of images continued for 4 h. Thereafter, the dishes were carefully washed once with fresh FluoroBrite DMEM without icFSP1 and refilled with medium. Then, image acquisition was re-started immediately. The images were recorded every 5 mins for one more hour, i.e. the total duration of data acquisition was around 5 h.

For determining the number of condensates in cells, Pfa1 cells (15,000-20,000 cells) were seeded on p-Slide 8 well (Ibidi, Cat #80826), and incubated overnight. On the next day, medium was changed to DMEM-high glucose supplemented with 10% FBS, 2 mM L-glutamine, and 1% penicillin/streptomycin, 2.5 μM icFSP1, and Hoechst. Immediately thereafter, focus was adjusted and Hoechst and EGFP images were recorded using an Axio Observer Z1 imaging system with VisView v4.0 (Visitron Systems, ZWISS) with 20× air objective and a CCD camera (CoolSnap ES2, Photometrics) with the corresponding filter sets. During imaging, the cells were maintained at 37° C. and 5% CO₂ atmosphere using a temperature-controlled incubation chamber. For visualizations, the imaging software ImageJ/Fiji was used and CellProfiler (v4.1.3, Broad Institute) was used for counting condensates per cells.

### 2.11 Tumor Tissue Staining

Dissected tissues were fixed in 4% paraformaldehyde in PBS overnight at 4° C. For immunofluorescence (IF) staining, fixed tissues were incubated in 20% sucrose in PBS overnight at 4° C., followed by embedding in OCT mounting compound (Tissue Tek, Sakura) on dry ice and stored at −80° C. The frozen tissues were cut in 5 μm thick sections using Cryostat Microm HM 560 (Thermo Fisher Scientific) at −30° C. Tissue sections were post-fixed with 1% paraformaldehyde in PBS for 10 min and subsequently fixed with 67% ethanol and 33% acetic acid for 10 min. Sections were incubated with blocking solution (5% goat serum, 0.3% Triton X-100 in PBS) for 30 min, and incubated with primary antibodies (anti-HA (clone: 3F10, 1:10, developed in-house), anti-4HNE (JaICA, Cat #HNEJ-2, 1:50), anti-AIFM2 (FSP1, clone:14D7, undilute, developed in-house)) diluted in blocking solution overnight at 4° C. On the next day, sections were incubated with appropriate fluorophore-conjugated secondary antibodies (goat anti-rat Alexa Fluor 488 IgG (H+L) (1:500, A-11006, Invitrogen), goat anti-mouse IgG H&L Alexa Fluor 647 (1:500, ab150115, Abcam), and donkey anti-rat IgG Alexa fluor 555 (1:500, ab150154, Abcam)) in secondary dilution buffer (1% BSA and 0.3% Triton X-100 in PBS) for 2 hours at room temperature. DNA was visualized by DAPI staining for 5 min, and slides were mounted in Aqua-/poly Mount. Images were obtained using LSM880 microscope (ZEISS) and analyzed by ZEN Blue or ImageJ/Fiji software.

### 2.12 Lentiviral Production and Transduction

HEK293T cells were used to produce lentiviral particles. The eco-tropic envelope protein of murine leukaemia virus (MLV) was used for mouse-derived cells, while the amphitropic envelope protein VSV-G was used for human-derived cells. A third-generation lentiviral packaging system consisting of transfer plasmids, envelope plasmids (pEcoEnv-IRES-puro or pHCMV-EcoEnv (ecotropic particles) or μMD2. G (pantropic particles)) and packaging plasmids (μMDLg pRRE and pRSV_Rev or psPAX2) was co-lipofected into HEK293T cells using transfection reagent (PEI MAX (Polysciences, cat. no. 24765) or X-tremeGENE HP reagent (Roche, cat. no. 06366236001)). Viral particle-containing cell culture supernatant was collected 48-72 h after transfection, filtered through a 0.45-μm PVDF filter (Millipore, cat. no. SLHV033RS) and then used for lentiviral transduction.

Cells were seeded on 12- or 6-well plates in medium supplemented with 10 μg ml-1 protamine sulfate and lentivirus was incubated with cells overnight. The next day, the cell culture medium was replaced with fresh medium containing appropriate antibiotics, such as puromycin (Gibco, cat. no. A11138-03; 1 μg ml-1), blasticidin (Invitrogen, cat. no. A1113903; 10 μg ml-1) or G418 (Invitrogen, cat. no. 10131-035; 1 mg ml-1)) and cells were cultured until non-transduced cells were dead.

### 2.13 CRISPR-Cas9-Mediated Gene Knockout

sgRNAs were designed to target critical exons of the genes of interest, and gene knockout was confirmed by western blotting. sgRNAs were cloned into BsmBI-digested lentiCRISPRv2-blast, lentiCRISPRv2-puro and lentiGuide-neo vectors (Addgene, cat. nos. 98293, 98290 and 139449). To generate knockout cells, MDA-MB-436, 786-0, A375, H460, B16F10 and 4T1 cells were transiently co-transfected with the desired sgRNAs expressed from lentiCRISPRv2-blast and lentiCRISPRv2-puro using X-tremeGENE HP reagent as described previously. One day after transfection, selection was started with puromycin (1 μg ml-1) and blasticidin (10 μg ml-1). After selection for 2-3 days, single-cell clones were isolated, and knockout clones were validated by immunoblotting and sequencing of genomic DNA. To generate Dox-inducible FSP1-EGFP-expressing cells, H460 FSP1KO cells were transduced with lentivirus (pCW-FSP1WT-EGFP-blast or pCW-FSP1Q319K-EGFP-blast). After Dox treatment of cells, scalable FSP1 expression was confirmed by immunoblotting.

### 2.14 Microsomal Stability

The objective of this study was to determine metabolic stability of 4 test articles and reference compounds in human and mouse liver microsomes at five time points over 40 minutes using HPLC-MS. Metabolic stability is defined as the percentage of parent compound lost over time in the presence of a metabolically active test system.

### 2.14.1 Reagents and Consumables

DMSO Chromasolv Plus, HPLC grade, ≥99.7% (Sigma-Aldrich, USA; Cat #34869) Acetonitrile Chromasolv, gradient grade, for HPLC, ≥99.9% (Sigma-Aldrich, USA; Cat #34851) Methanol, HiPerSolv, HPLC-gradient grade, ≥99.9% (VWR Chemicals, USA, Cat #20864.320) Potassium phosphate monobasic (Bio-Basic, Canada; Lot #N9016010) Potassium phosphate dibasic (Bio-Basic, Canada; Lot #MA7100050) Magnesium chloride hexahydrate (Santa Cruz Biotechnology, Inc., USA; sc-203126A) Human Liver Microsomes: pooled, mixed gender (XenoTech, H0630/lot N #1210097) Mouse Liver Microsomes: pooled, male Balb/c mice (XenoTech, M3000/lot #2010026) Glucose-6-phosphate dehydrogenase from baker's yeast, type XV (Sigma-Aldrich, USA; Cat #G6378) D-Glucose-6-phosphate sodium salt (Sigma-Aldrich, USA; Cat #G7879-1G) NADPH tetrasodium salt (BLD Pharmatech Ltd., Cat #BD116582) Formic acid (Sigma-Aldrich, 94318) Verapamil hydrochloride (Sigma Aldrich, USA; Cat #V4629) Niclosamide (Sigma-Aldrich, USA; Cat #N3510) DMSO stock solutions of the tested compounds 20 mM (+, −) Propranolol hydrochloride (Sigma-Aldrich, P0884) Imipramine hydrochloride (Sigma-Aldrich, 17379) Diclofenac, 96% purity (Enamine, #EN300-119509) Phenomenex Luna® C18 HPLC column, 2.1×50 mm, 5 μm (Cat #5291-126) Phenomenex Luna® C18 HPLC column, 2×30 mm, 5 μm (S. N. 146953-2) Matrix™ 0.75 ml blank tubes (Cat #4170), pipettor tips (Thermo Scientific).

### 2.14.2 Equipment

Gradient HPLC system (Shimadzu) Triple quadrupole mass-detector API 3000 with TurbolonSpray Ion Source (AB Sciex, Canada) Nitrogen generator N2-04-L1466, nitrogen purity 99%+(Whatman) Environmental Incubator Shaker G24; Digital Refrigerated Incubator/Shaker Innova 4330 (New Brunswick Scientific) Water purification system Millipore Milli-Q Gradient A10 (Millipore, France) Multichannel pipettors 1-30 μL, 2-125 μL, 30-850 μL (Thermo Scientific)

### 2.14.3 Analytical System

All measurements were performed using Shimadzu HPLC system including vacuum degasser, gradient pumps, reverse phase HPLC column, column oven, and autosampler. Mass spectrometric analysis was performed using a Triple quadrupole mass-detector API 3000 with TurbolonSpray Ion Source (AB Sciex, Canada) with Turbo V ion source and Turbolonspray interface. The TurbolonSpray ion source was used in both positive and negative ion modes. The data acquisition and system control was performed using Analyst 1.6.3 software from AB Sciex.

### 2.14.4. Methods

Microsomal incubations were carried out in 96-well plates in 5 aliquots of 30 μL each (one for each time point). Liver microsomal incubation medium comprised of phosphate buffer (100 mM, pH 7.4), MgCl₂ (3.3 mM), NADPH (3 mM), glucose-6-phosphate (5.3 mM), glucose-6-phosphate dehydrogenase (0.67 units/ml) with 0.42 mg of liver microsomal protein per ml. In the control reactions, the NADPH-cofactor system was substituted with phosphate buffer. Test compounds (2 μM, final solvent concentration 1.6%) were incubated with microsomes at 37° C., shaking at 100 rpm. Five time points over 40 minutes were analyzed. The reactions were stopped by adding 5 volumes of acetonitrile with internal standard to incubation aliquots, followed by protein sedimentation by centrifuging at 5500 rpm for 5 minutes. Each reaction was performed in duplicates. Supernatants were analyzed using the HPLC system coupled with a tandem mass spectrometer.

The elimination constant (kel), half-life (t_{1,2}), and intrinsic clearance (Clᵢₙₜ) were determined in a plot of In(AUC) versus time, using linear regression analysis:¹

$${k}_{el} = - slope$$

$${t}_{1 / 2} = \frac{0.693}{k}$$

$${Cl}_{int} = \frac{0.693}{{t}_{1 / 2}} \times \frac{{μl}_{incubation}}{{mg}_{microsomes}}$$

1 In order to indicate the quality of the linear regression analysis, the R2 (determination coefficient) values are provided. In some cases, the last time point is excluded from the calculations to ensure acceptable logarithmic linearity of decay

### 2.14.5. Interpretation of Microsomal Stability Assay Data

The test compounds can be classified in terms of their microsomal stability into low, medium and high clearance groups. Intrinsic Clearance (in vitro) can be recalculated to Intrinsic Clearance (in vivo) using literature data for liver weight and liver blood flow with the next equation²⁴:

$$Predicted in vivo {CL}_{int , u} = \frac{in vitro {CL}_{int} \times PBSF \times LW}{{f}_{u mic} or {f}_{u heps}}$$

where,
in vivo CLᵢₙₜ—predicted in vivo intrinsic clearance, mL/min/kg in vitro CLᵢₙₜ—in vitro microsomal clearance, mL/min/mg
PBSF—physiologically based scaling factor—the microsomal average recovery factor for microsomal predictions and hepatocellularity for hepatocyte predictions, mg/g
LW—liver weight/kg bodyweight, g/kg
f_{u mic} or f_{u heps}—fraction unbound in either microsomes or hepatocytes (can be determined from Plasma Protein Binding study or assumed as, if it is unknown)
Using in vivo Clint hepatic clearance can be predicted based on a “well-stirred” liver model using the next formula²⁵:

$${CL}_{H} = \frac{{Q}_{H} \times {f}_{u} \times {CL}_{int}}{{Q}_{H} \times {f}_{u} \times {CL}_{int}}$$

where,
CLH—predicted hepatic clearance, mL/min/kg
QH—liver blood flow, mL/min/kg
fu—fraction unbound in the blood
CLᵢₙₜ—predicted in vivo clearance, mL/min/kg
The CLᵢₙₜ classification values were calculated for mouse, rat, and human species using the literature data on liver weight³ and microsomal protein concentration^{26,27} and are represented in the following table.

TABLE 3
The intrinsic clearance groups for classification of test compounds

| | |
|---|---|
| Classification | Intrinsic clearance (μL/min/mg protein) |

| Group | Mouse | Rat | Human |
|---|---|---|---|
| Low clearance | <8.6 | <13 | <8.8 |
| High clearance | >48 | >72 | >48 |

### 2.14.6 Results

The results of the microsomal stability measurements are provided in table 4.

TABLE 4
Microsomal stability measurements

| | Mouse liver | Human liver |
|---|---|---|
| | microsomes: | microsomes: |
| Compound | CLint | CLint |
| Number | (μL/min/mg) | (μL/min/mg) |
| 74 | 7 | 7 |
| 79 | 4 | 5 |
| 13 | 6 | 12 |
| 82 | 15 | 4 |

### 2.15. Pharmacokinetics

### 2.15.1 Study Objective

The purpose of this study was to determine the pharmacokinetic characteristics of compound 13 in male Balb/cAnN mice following intraperitoneal (IP) administration. Levels of the test compound were determined by LC-MS/MS in the blood plasma samples over time after a single dose.

### 2.15.2 Reagents and Consumables

DMSO Chromasolv Plus, HPLC grade, ≥99.7% (Sigma-Aldrich, USA; Cat #34869);
Acetonitrile Chromasolv, gradient grade, for HPLC, ≥99.9% (Sigma-Aldrich, USA; Cat #34851);
Methanol Chromasolv Plus, for HPLC, ≥99.9% (Sigma-Aldrich, USA; Cat 34860);
Formic acid for mass spectrometry, −98% (Fluka, USA; Cat #94318);
DMSO pharma grade, ≥99.9% (Pharma grade. PanReact Applichem, Germany; Cat #191954.1611);
(2-hydroxypropyl)-β-cyclodextrin (L'eternel World, LLC; Purity 99.5%);
Water for injections (“Arterium”, Ukraine, Lot #233142);
Sodium hydroxide (Bio Basic Canada Inc., Canada, Cat #A620617-0500);
Microtainer Blood Collection Tubes K₃EDTA, Henso, Lot #191010;
2,2,2-Tribromoethanol 97% (Sigma-Aldrich; Cat #T48402);
Amyl alcohol (UOS, Ukraine);
Tubes (Falcon, 5 ml, 12×75 mm, USA);
Tubes (Eppendorf, 1.5 ml);
Syringes (BD, 1 ml, tuberculin slip tip, USA, REF 3096).
Compound Prometryn was used as an internal standard (IS).
Compound FS-30 (13) was supplied as dry powder. The vehicle was DMSO—40% 2HPPCD in Water for injection, w/v (10%:90%, v/v). Preparation of the formulation was carried out under “red light”.To prepare the formulation, 0.3 ml of DMSO was added to the compound (6 mg); the mixture was vortexed for 10 sec—clear pink solution. Next, 2 ml of 40% 2HPPCD aqueous solution was added to the formulation; the mixture was vortexed for 10 sec—clear pink solution (pH 4.07). After that, the formulation was neutralized with 2 μL of 1M NaOH, and 0.698 ml of 40% 2HPβCD aqueous solution was added to the formulation; the mixture was vortexed for 10 sec—clear yellow solution (pH 7.29)
The batches of working formulations were prepared 5 min prior to the in vivo study.

### 2.15.3 Equipment

Gradient HPLC system (Shimadzu, Japan);
MS/MS detector API 3000 with TurbolonSpray Electrospray module (AB Sciex, Canada);
IMT-PN 1280 OG Nitrogen Generator (INMATEC Technologies GmbH, Germany);
Water purification system Arium mini (Sartorius, Germany);
VWR Analog Vortex Mixer VM 3000 (VWR, USA);
Centrifuge 4-15C (Qiagen) (Sigma, Germany).

### 2.15.4 Study Design

Study design, animal selection, handling and treatment were all in accordance with the Enamine PK study protocols and Institutional Animal Care and Use Guidelines (BACUC approval number #HC-PK-10112023). Animal treatment and samples preparation were conducted by the Animal Laboratory personnel at Enamine/Bienta. Male Balb/cAnN mice (10 weeks old, body weight ranged from 19.6 g to 23.3 g and average body weight across all groups 21.0 g, SD=0.9 g) were used in this study. The animals were randomly assigned to the treatment groups before the pharmacokinetic study; all animals were fasted for 4 h before dosing. Six sampling time points (5, 15, 30, 60, 120, and 360 min) were set for this pharmacokinetic study. Each of the time point treatment group included 4 animals. There was also a control group of one vehicle-dosed animal. Dosing was done according to the treatment Schedules outlined in Table 5. Mice were injected IP with 2,2,2-tribromoethanol at the dose of 150 mg/kg prior to drawing the blood. Blood collection was performed from the orbital sinus in microtainers containing K₃EDTA. Animals were sacrificed by cervical dislocation after the blood samples collection. Blood samples were centrifuged for 10 min at 3000 rpm. All samples were immediately processed, flash-frozen and stored at −70° C. until subsequent analysis.

TABLE 5
Study design

| | | | | Target | | Target |
|---|---|---|---|---|---|---|
| | | | | Dose | Target Dose | Dose |
| Number | | | Delivery | Level | Concentration | Volume |
| of Animals | Compound ID | Formulation | Route | (mg/kg) | (mg/ml) | (ml/kg) |
| 24 | 13 | DMSO - 40% | IP | 10 | 0.5 | 20 |
| 1 | Vehicle | 2HPβCD in Water | IP | 0 | 0 | 20 |
| | dosed | for injection, w/v | | | | |
| | | (10%:90%, v/v) | | | | |

### 2.15.5 Samples Processing

Plasma samples (40 μl) were mixed with 200 μl of IS(90) solution. After mixing by pipetting and centrifuging for 4 min at 6000 rpm, 0.25 μl of each supernatant was injected into LC-MS/MS system.

Solution of Prometryn (200 ng/ml in water-methanol mixture 1:9, v/v) was used as internal standard (IS(90)) for the quantification of 13 in plasma samples.

### 2.15.6 Samples Analysis

Analyses of plasma samples were conducted by the Bioanalytical Laboratory personnel at Enamine/Bienta. The concentrations of 13 in samples were determined using high performance liquid chromatography/tandem mass spectrometry (HPLC-MS/MS). Shimadzu HPLC system comprised 2 isocratic pumps LC-10ADvp, an autosampler SIL-20AC, a sub-controller FCV-14AH and a degasser DGU-14A. Mass spectrometric analysis was performed using an API 3000 (triple-quadrupole) instrument from AB Sciex (Canada) with an electro-spray (ESI) interface. The data acquisition and system control were performed using Analyst 1.6.3 software from AB Sciex.

### 2.15.7 HPLC-MS/MS Conditions

### Chromatographic Conditions:

Column: Synergi Hydro-RP 80A, 2×30 mm, 4 μm
Mobile phase A: Acetonitrile: Water: Formic acid=50:950:1
Mobile phase B: Acetonitrile: Formic acid=100:0.1
Linear gradient: 0 min 10% B, 1.0 min 100% B, 1.2 min 100% B, 1.21 min 10% B, 2.4 min stop
Elution rate: 400 μL/min. A divert valve directed the flow to the detector from 1.5 to 1.8 min
Column temperature: 30° C.

### MS/MS Detection:

Scan type: Positive MRM, Ion source: Turbo spray, Ionization mode: ESI
Nebulize gas: 15 L/min, Curtain gas: 8 L/min, Collision gas: 4 L/min
Ionspray voltage: 5000 V, Temperature: 400C

TABLE 6
Other MS parameters

| | Parent, | Daughter, | Time, | DP, | FP, | EP, | CE, | CXP, |
|---|---|---|---|---|---|---|---|---|
| Compound ID | m/z | m/z | ms | V | V | V | V | V |
| 13 | 370.195 | 251.100 | 70 | 76 | 280 | 11 | 47 | 10 |
| Prometryn | 242.100 | 158.00 | 70 | 36 | 230 | 11 | 35 | 8 |

### 2.15.8 Preparation of Calibration Standards

Calibration standards for quantification of 13 in plasma samples. Compound 13 was dissolved in DMSO, and the resulting solution with a concentration of 2 mg/ml was used for calibration standards preparation (stock solution). The stock solution was consecutively diluted with IS(90) to get a series of calibration solutions with final concentrations of 10 000, 4 000, 2 000, 1 000, 400, 200, 100, 40, and 20 ng/ml. The calibration curve was constructed using blank mouse plasma samples. To obtain calibration standards, blank plasma samples (40 μl) were mixed with 200 μl of the corresponding calibration solution. After mixing by pipetting and centrifuging for 4 min at 6000 rpm, 0.25 μl of each supernatant was injected into LC-MS/MS system.

### 2.15.9 Pharmacokinetic Method Analysis

The concentrations of the test compound below the lower limit of quantitation (LLOQ=100 ng/ml) were designated as zero. The pharmacokinetic data analysis was performed using noncompartmental, bolus injection or extravascular input analysis models in WinNonlin 5.2 (PharSight). Data below LLOQ were presented as missing to improve the validity of T½ calculations.

For each treatment condition, the final concentration values obtained at each time point were analyzed for outliers using Grubbs' test with the level of significance set at p<0.05.

### 2.15.10 Results

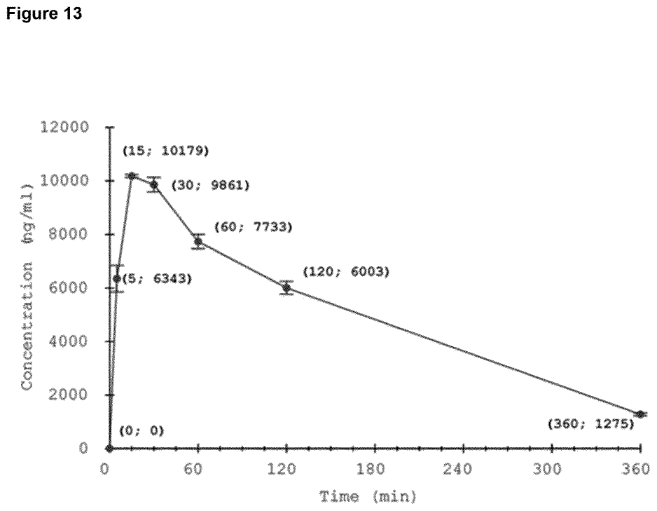

The pharmacokinetic parameters of compound compound 13 were calculated according to non-compartmental analysis using the WinNonlin program as illustrated in Table 7. The corresponding plasma-concentration time curve is shown in FIG. 13. Based on the total drug concentration, the plasma concentration still exceeds the EC₅₀ value by 10-fold in vivo.

TABLE 7
Selected calculated pharmacokinetic parameters of compound 13

| | | Pharmacokinetic Parameters | | | | |
|---|---|---|---|---|---|---|
| | | | | | AUC_{0→t = 60 min} | AUC_{0→∞} |
| | administration | dose | Tmax, | Cmax, | (AUClast) | (AUCINF_obs), |
| species | route | (mg/kg) | (min) | (ng/ml) | (ng*min/ml) | (ng*min/ml) |
| mouse | IP | 10 | 15.0 | 10200 | 1800000 | 2010000 |

| | Pharmacokinetic Parameters | | | |
|---|---|---|---|---|
| | T_{1/2} | Kₑₗ | MRT | MRT |
| | (HL_Lambda_z), | (Lambda_z), | (MRTlast), | (MRTinf), |
| species | (min) | (min⁻¹) | (min) | (min) |
| mouse | 114 | 0.00608 | 107 | 151 |

### REFERENCES

1. Yang, W. S., SriRamaratnam, R., Welsch, M. E., Shimada, K., Skouta, R., Viswanathan, V. S., Cheah, J. H., Clemons, P. A., Shamji, A. F., Clish, C. B., et al. (2014). Regulation of ferroptotic cancer cell death by GPX4. Cell 156, 317-331. 10.1016/j.cell.2013.12.010.
2. Friedmann Angeli, J. P., Schneider, M., Proneth, B., Tyurina, YY, Tyurin, V. A., Hammond, V. J., Herbach, N., Aichler, M., Walch, A., Eggenhofer, E., et al. (2014). Inactivation of the ferroptosis regulator Gpx4 triggers acute renal failure in mice. Nature cell biology 16, 1180-1191. 10.1038/ncb3064.
3. Ingold, I., Berndt, C., Schmitt, S., Doll, S., Poschmann, G., Buday, K., Roveri, A., Peng, X., Porto Freitas, F., Seibt, T., et al. (2018). Selenium Utilization by GPX4 Is Required to Prevent Hydroperoxide-Induced Ferroptosis. Cell 172, 409-422 e421. 10.1016/j.cell.2017.11.048.
4. Conrad, M., Angeli, J. P., Vandenabeele, P., and Stockwell, B. R. (2016). Regulated necrosis: disease relevance and therapeutic opportunities. Nature reviews. Drug discovery 15, 348-366. 10.1038/nrd.2015.6.
5. Zheng, J., and Conrad, M. (2020). The Metabolic Underpinnings of Ferroptosis. Cell Metab 32, 920-937. 10.1016/j.cmet.2020.10.011.
6. Jiang, X., Stockwell, B. R., and Conrad, M. (2021). Ferroptosis: mechanisms, biology and role in disease. Nature reviews. Molecular cell biology 22, 266-282. 10.1038/s41580-020-00324-8.
7. Conrad, M., Lorenz, S. M., and Proneth, B. (2021). Targeting Ferroptosis: New Hope for As-Yet-Incurable Diseases. Trends Mol Med 27, 113-122. 10.1016/j.molmed.2020.08.010.
8. Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072. 10.1016/j.cell.2012.03.042.
9. Angeli, J. P. F., Shah, R., Pratt, D. A., and Conrad, M. (2017). Ferroptosis Inhibition: Mechanisms and Opportunities. Trends in pharmacological sciences 38, 489-498. 10.1016/j.tips.2017.02.005.
10. Larraufie, M. H., Yang, W. S., Jiang, E., Thomas, A. G., Slusher, B. S., and Stockwell, B. R. (2015). Incorporation of metabolically stable ketones into a small molecule probe to increase potency and water solubility. Bioorganic & medicinal chemistry letters 25, 4787-4792. 10.1016/j.bmcl.2015.07.018.
11. Cramer, S. L., Saha, A., Liu, J., Tadi, S., Tiziani, S., Yan, W., Triplett, K., Lamb, C., Alters, S. E., Rowlinson, S., et al. (2017). Systemic depletion of L-cyst(e)ine with cyst(e)inase increases reactive oxygen species and suppresses tumor growth. Nature medicine 23, 120-127. 10.1038/nm.4232.
12. Eaton, J. K., Ruberto, R. A., Kramm, A., Viswanathan, V. S., and Schreiber, S. L. (2019).

Diacylfuroxans Are Masked Nitrile Oxides That Inhibit GPX4 Covalently. Journal of the American Chemical Society 141, 20407-20415. 10.1021/jacs.9b10769.
13. Eaton, J. K., Furst, L., Ruberto, R. A., Moosmayer, D., Hilpmann, A., Ryan, M. J., Zimmermann, K., Cai, L. L., Niehues, M., Badock, V., et al. (2020). Selective covalent targeting of GPX4 using masked nitrile-oxide electrophiles. Nature chemical biology 16, 497-506. 10.1038/s41589-020-0501-5.
14. Mei, J., Webb, S., Zhang, B., and Shu, H. B. (2006). The p53-inducible apoptotic protein AMID is not required for normal development and tumor suppression. Oncogene 25, 849-856. 10.1038/sj.onc.1209121.
15. Tonnus, W., Meyer, C., Steinebach, C., Belavgeni, A., von Massenhausen, A., Gonzalez, N. Z., Maremonti, F., Gembardt, F., Himmerkus, N., Latk, M., et al. (2021). Dysfunction of the key ferroptosis-surveilling systems hypersensitizes mice to tubular necrosis during acute kidney injury. Nature communications 12, 4402. 10.1038/s41467-021-24712-6.
16. Doll, S., Freitas, F. P., Shah, R., Aldrovandi, M., da Silva, M. C., Ingold, I., Goya Grocin, A., Xavier da Silva, T. N., Panzilius, E., Scheel, C. H., et al. (2019). FSP1 is a glutathione-independent ferroptosis suppressor. Nature 575, 693-698. 10.1038/s41586-019-1707-0.
17. Zheng, J., Sato, M., Mishima, E., Sato, H., Proneth, B., and Conrad, M. (2021). Sorafenib fails to trigger ferroptosis across a wide range of cancer cell lines. Cell death & disease 12, 698. 10.1038/s41419-021-03998-w.
18. Schmitt, A., Xu, W., Bucher, P., Grimm, M., Konantz, M., Horn, H., Zapukhlyak, M., Berning, P., Brandle, M., Jarboui, M. A., et al. (2021). Dimethyl fumarate induces ferroptosis and impairs NF-kappaB/STAT3 signaling in DLBCL. Blood 138, 871-884. 10.1182/blood.2020009404.
19. Seiler, A., Schneider, M., Forster, H., Roth, S., Wirth, E. K., Culmsee, C., Plesnila, N., Kremmer, E., Radmark, O., Wurst, W., et al. (2008). Glutathione peroxidase 4 senses and translates oxidative stress into 12/15-lipoxygenase dependent- and AlF-mediated cell death. Cell Metab 8, 237-248. 10.1016/j.cmet.2008.07.005.
20. Bersuker, K., Hendricks, J. M., Li, Z., Magtanong, L., Ford, B., Tang, P. H., Roberts, M. A., Tong, B., Maimone, T. J., Zoncu, R., et al. (2019). The CoQ oxidoreductase FSP1 acts parallel to GPX4 to inhibit ferroptosis. Nature 575, 688-692. 10.1038/s41586-019-1705-2.
21. Pontel, L. B., Bueno-Costa, A., Morellato, A. E., Carvalho Santos, J., Roué, G., and Esteller, M. (2022). Acute lymphoblastic leukemia necessitates GSH-dependent ferroptosis defenses to overcome FSP1-epigenetic silencing. Redox biology 55, 102408. https://doi.org/10.1016/i.redox.2022.102408.
22. Nakamura, T., Ogawa, M., Kojima, K., Takayanagi, S., Ishihara, S., Hattori, K., Naguro, I., and Ichijo, H. (2021). The mitochondrial Ca2+ uptake regulator, MICU1, is involved in cold stress-induced ferroptosis. EMBO reports 22, e51532-e51532. 10.15252/embr.202051532.
23. Mishima, E., Ito, J., Wu, Z., Nakamura, T., Wahida, A., Doll, S., Tonnus, W., Nepachalovich, P., Eggenhofer, E., Aldrovandi, M., et al. (2022). A non-canonical vitamin K cycle is a potent ferroptosis suppressor. Nature. 10.1038/s41586-022-05022-3.
24. Wood, F. L., Houston, J. B., & Hallifax, D. (2017). Clearance prediction methodology needs fundamental improvement: Trends common to rat and human hepatocytes/microsomes and implications for experimental methodology. Drug Metabolism and Disposition, 45(11), 1178-1188. https://doi.org/10.1124/dmd.117.077040
25. Laveé, T., & Funk, C. (2007). In vivo absorption, distribution, metabolism, and excretion studies in Discovery and Development. Comprehensive Medicinal Chemistry II, 31-50. https://doi.org/10.1016/bO-08-045044-x/00118-8
26. Barter Z. E., et al., Scaling factors for the extrapolation of in vivo metabolic drug clearance from in vitro data: reaching a consensus on values of human microsomal protein and hepatocellularity per gram of liver, Current Drug Metabolism, 2007, 8, 33-45.
27. Iwatsubo T., et al., Prediction of species differences (rats, dogs, humans) in the in vivo metabolic clearance of YM796 by the liver from in vitro data, Journal of Pharmacology and Experimental Therapeutics, 1997, 283, 462-469.

1. A compound according to formula (I) or (II), preferably formula (I) for use in the treatment of cancer, 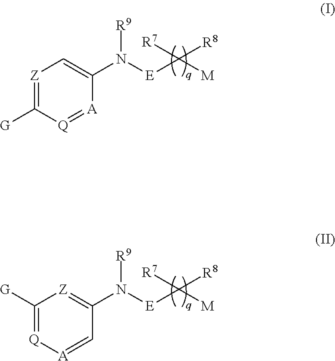
wherein G is selected from the group consisting of 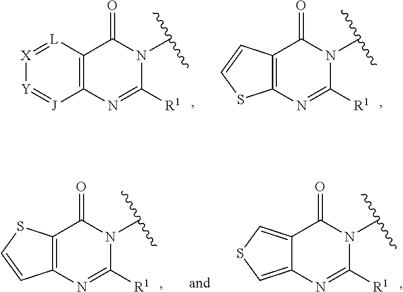
preferably 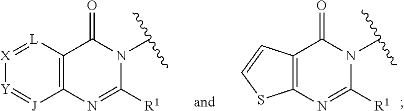
M is selected from the group consisting of 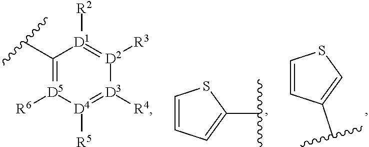
preferably 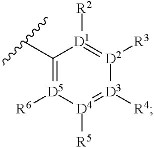
Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
L is selected from the group consisting of CH, N, C—F, C—Cl, and C—(C₁-C₆)alkyl, preferably CH;
X is selected from the group consisting of CH, N, C—F, C—Cl, and C—(C₁-C₆)alkyl, preferably CH;
Y is selected from the group consisting of CH, N, C—F, C—Cl, and C—(C₁-C₆)alkyl, preferably CH;
X and Y may be part of ring according to formula (III): 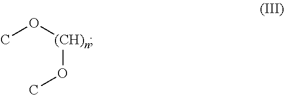
n is an integer from 1 to 3;
J is selected from the group consisting of CH, N, C—F, C—Cl, C—(C₁-C₆)alkyl, preferably CH;
D¹, D², D³, D⁴, D⁵, are independently selected from the group consisting of C and N;
R¹ is selected from the group consisting of —(C₁-C₆)alkyl, —(C₃-C₆)cycloalkyl, —CH₂OH, —CH₂F, —CHF₂, and —CF₃, preferably —(C₁-C₆)alkyl;
R² is selected from the group consisting of H, —(C₁-C₆)alkyl, —CF₃, —Cl, and —F, preferably H;
R³ is selected from the group consisting of H, —(C₁-C₆)alkyl, —CF₃, —O(C₁-C₆)alkyl, —Cl, and —F, preferably —O(C₁-C₆)alkyl;
R⁴ is selected from the group consisting of H, —(C₁-C₆)alkyl, —O(C₁-C₆)alkyl, —CF₃, —Cl, and —F, preferably —O(C₁-C₆)alkyl;
R⁵ is selected from the group consisting of H, —(C₁-C₆)alkyl, —O(C₁-C₆)alkyl, —CF₃, —C, and —F, preferably —O(C₁-C₆)alkyl;
R⁶ is selected from the group consisting of H, —(C₁-C₆)alkyl, —CF₃, —Cl, and —F, preferably H;
two groups of R², R³, R⁴, R⁵ and R⁶ in vicinal position may be part of a ring selected from 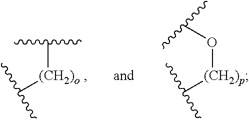
o is an integer between 1 and 4;
p is an integer between 1 and 4;
q is 1 or 0;
R⁷ and R⁸ are independently selected from the group consisting of H, and —(C₁-C₆)alkyl;
R⁷ and R⁸ may form a 3 to 5 membered cycloalkyl ring;
R⁹ is selected from the group consisting of H, and —(C₁-C₆)alkyl or
a pharmaceutically acceptable salt thereof.

2. A pharmaceutical composition comprising a compound as defined in claim 1 and at least one pharmaceutically acceptable excipient for use in the treatment of cancer.

3. The compound for use of claim 1 or the pharmaceutical composition for use of claim 2, wherein the cancer is a cancer which expresses ferroptosis suppressor protein-1 (FSP1).

4. The compound for use of claim 1 or the pharmaceutical composition for use of claim 2, wherein the cancer is selected from the group consisting prostate cancer, leukemia, liver cancer, breast cancer, hepatocellular carcinoma, cholangiocarcinoma, glioblastoma, uveal melanoma, adrenocortical cancer, thymoma, head and neck squamous cell carcinoma, cholangiocarcinoma, kidney cancer, lymphoid neoplasm diffuse large B-cell lymphoma, pancreatic adenocarcinoma, gallbladder cancer, lymphoma, myeloma, gastric cancer, brain cancer, skin cancer, colon/colorectal cancer, bile duct cancer, neuroblastoma, bone cancer, and lung cancer.

5. The compound for use of claim 4 or the pharmaceutical composition for use of claim 4, wherein
a) leukemia is selected from acute myeloid leukemia, acute lymphocytic leukemia, and chronic myeloid leukemia; and/or b) kidney cancer is selected from kidney clear cell carcinoma, and renal cell carcinoma; and/or
c) lung cancer is selected from small cell lung cancer, non small cell lung cancer, mesothelioma.

6. The compound for use of claim 1, and 3 to 5 or the pharmaceutical composition according to claim 2 to 5, wherein the compound is selected from the group consisting of

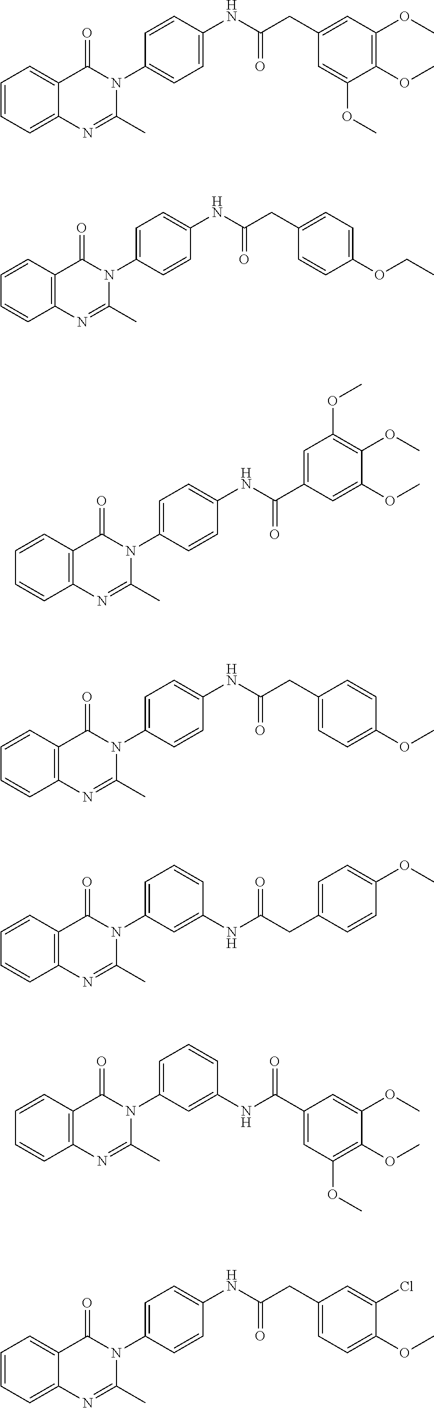

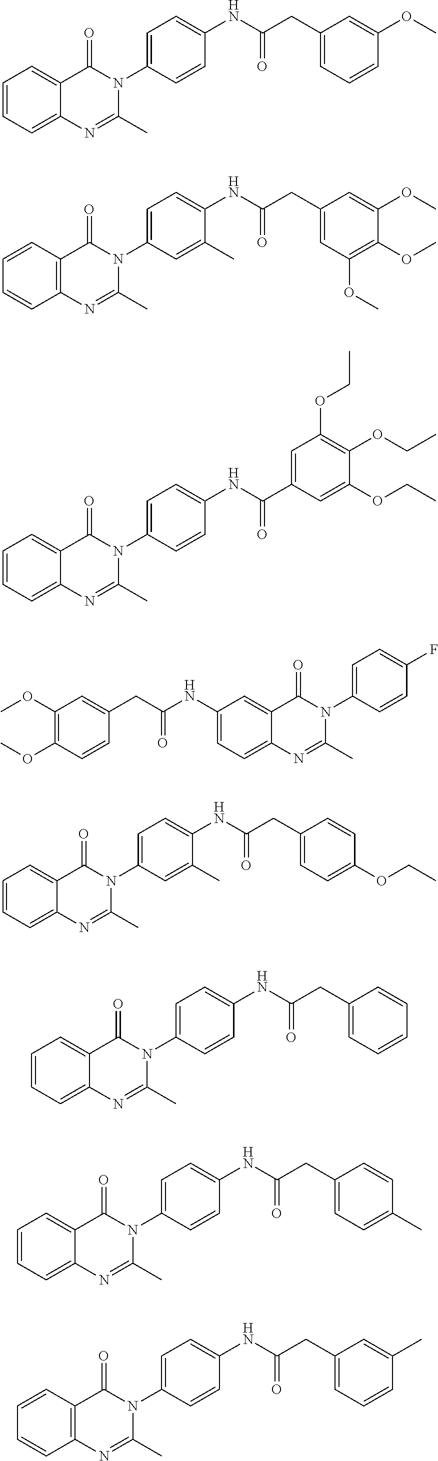

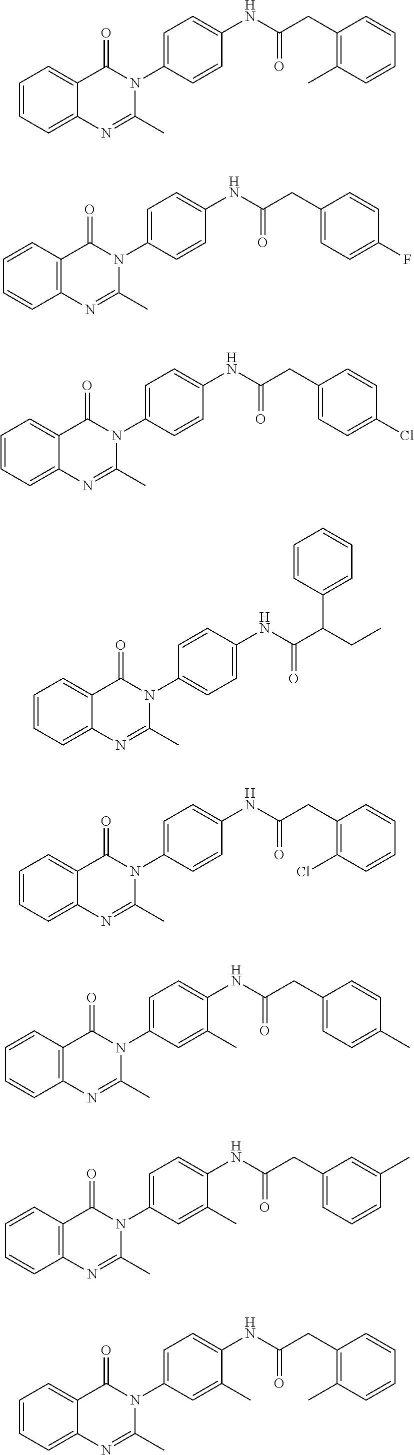

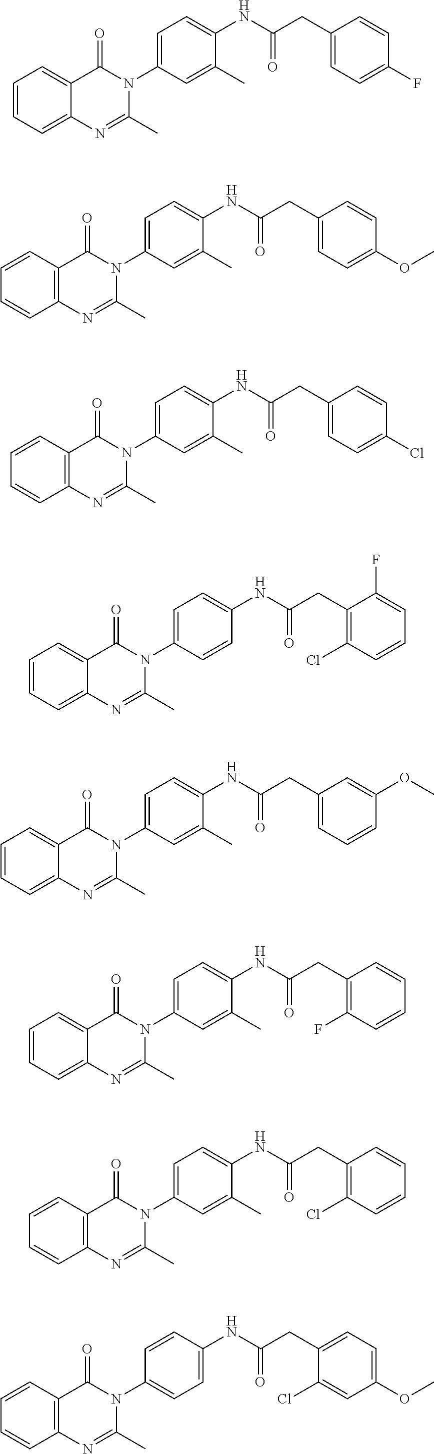

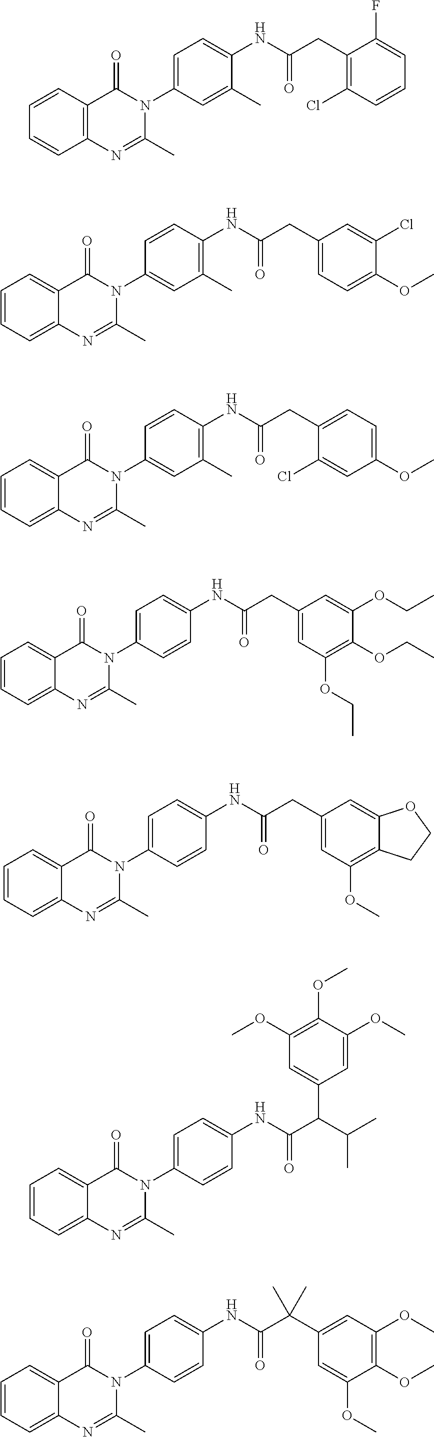

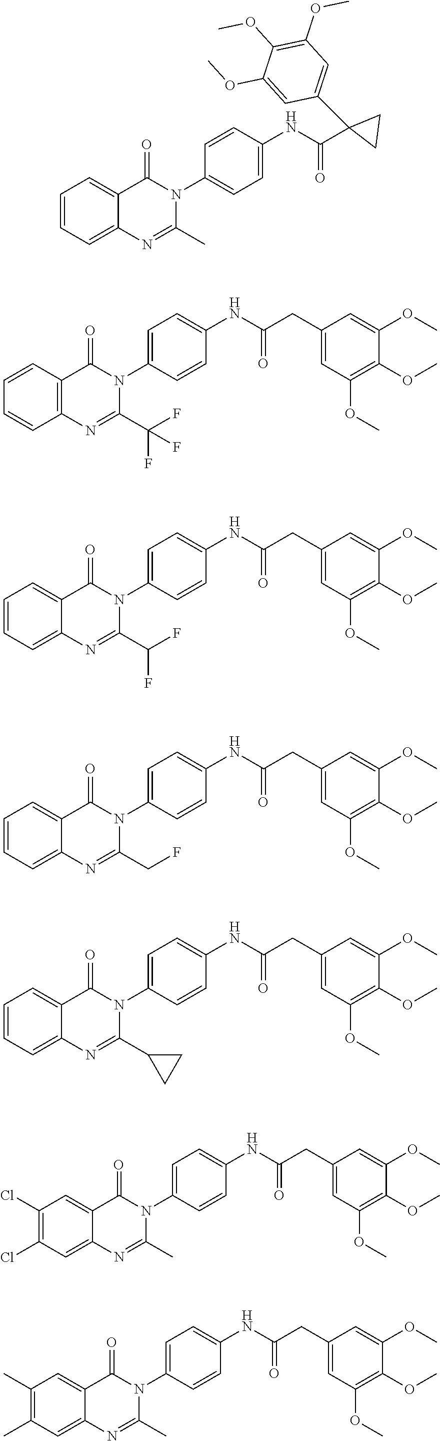

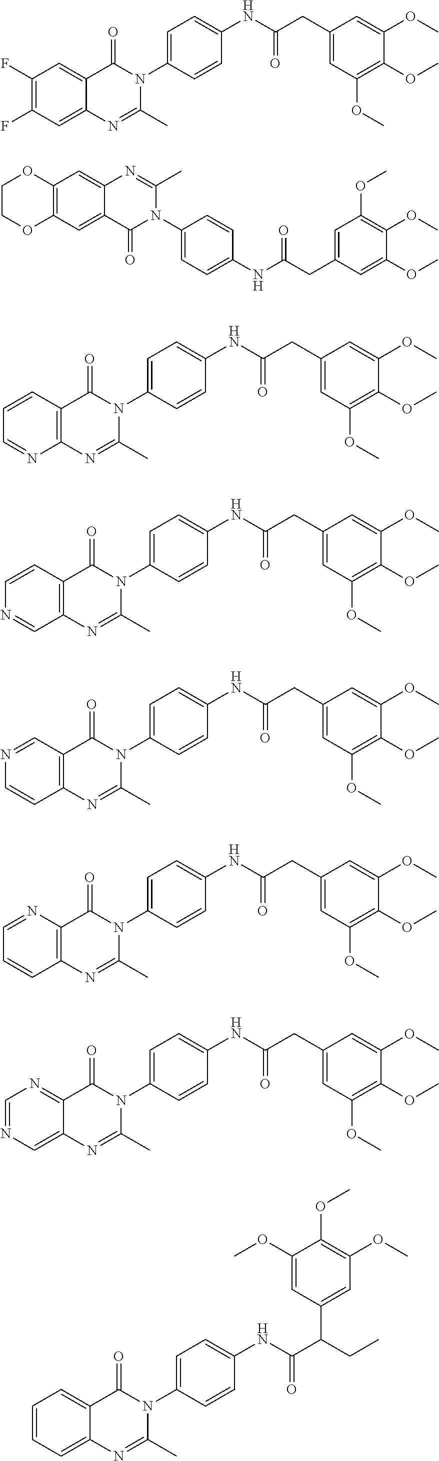

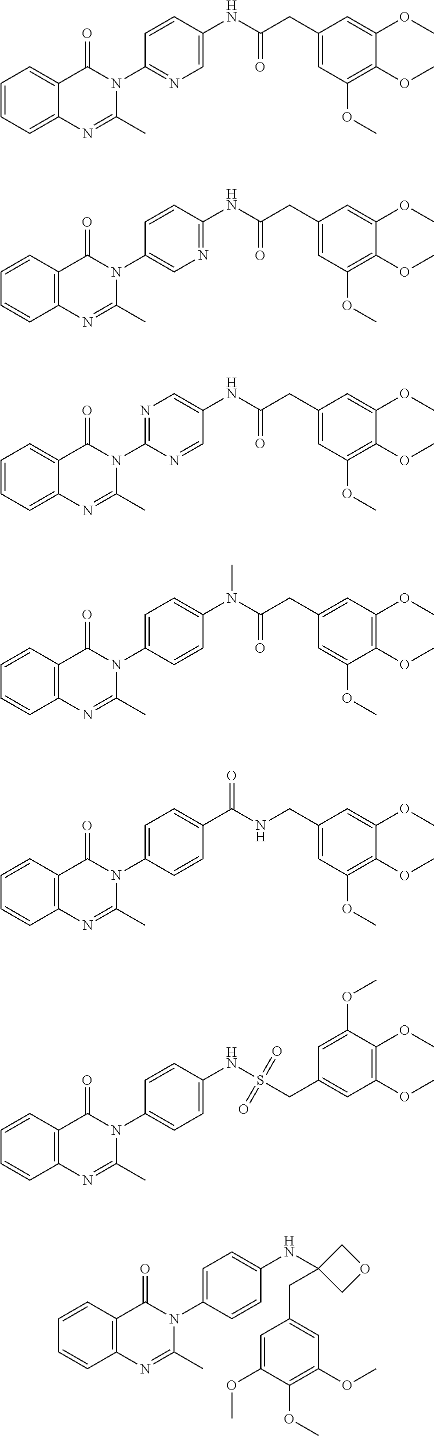

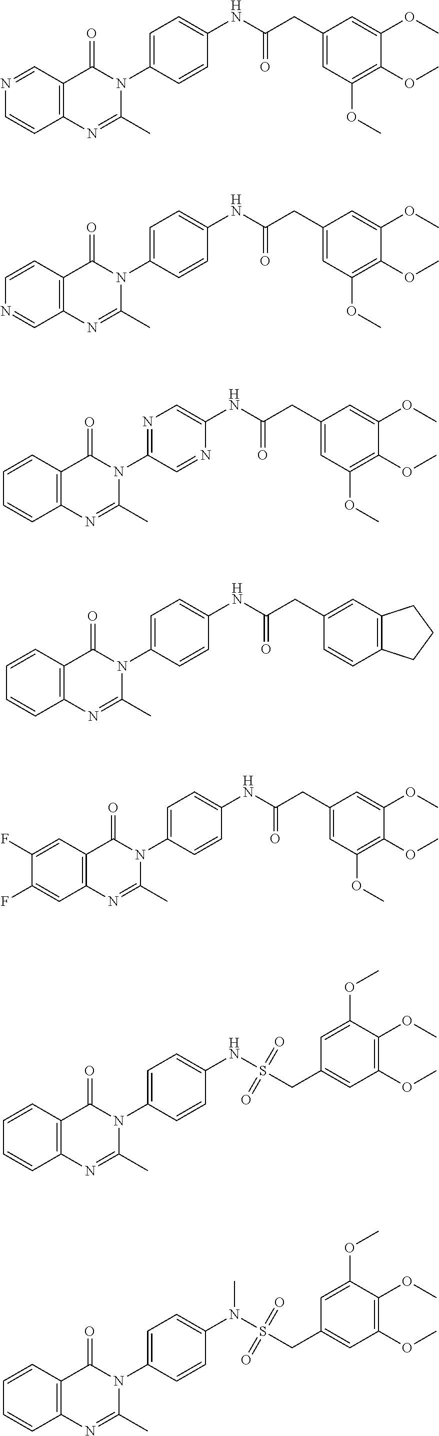

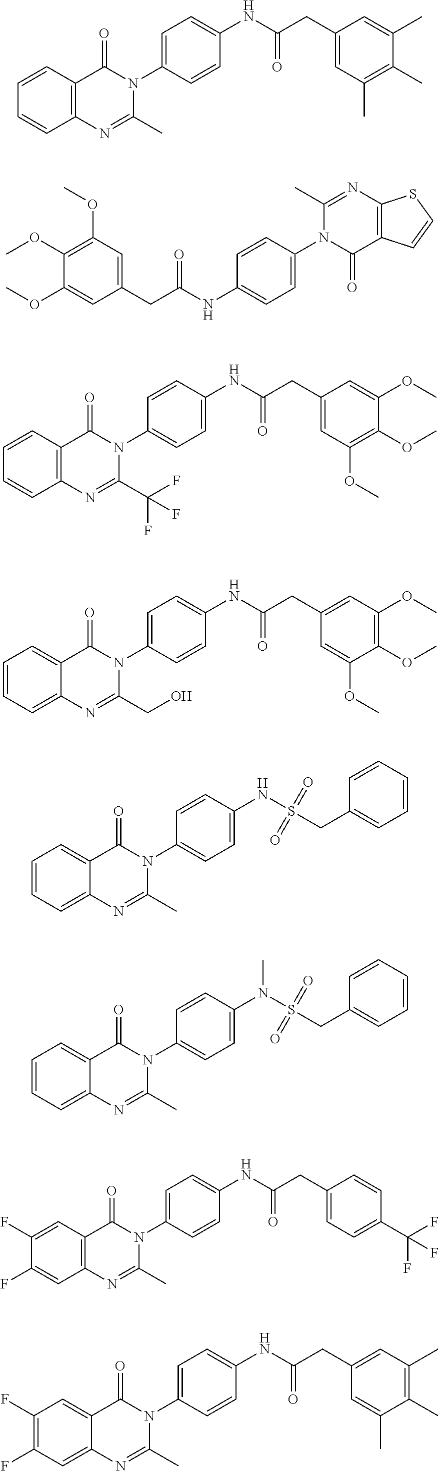

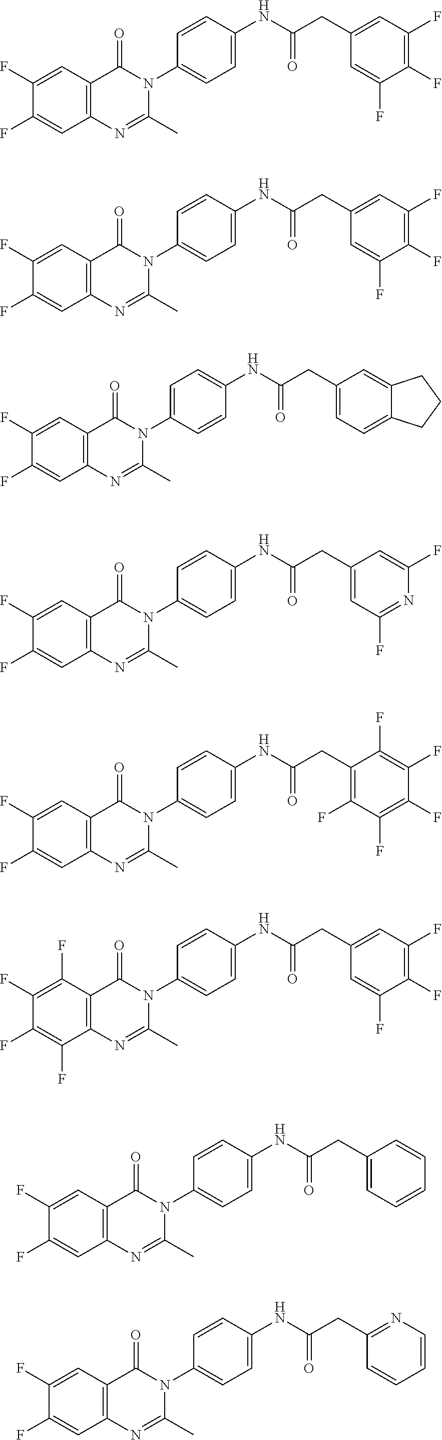

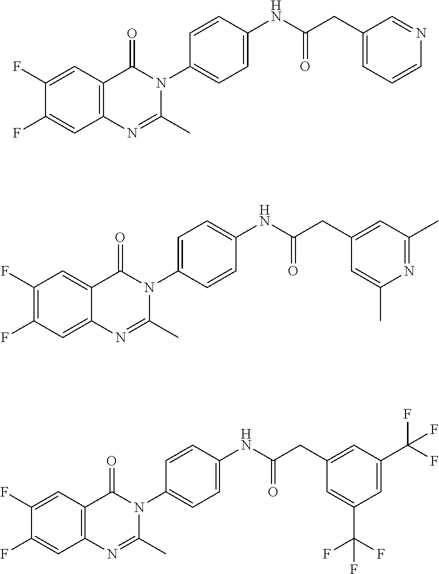 [SMILES: Cc1nc2cc(F)c(F)cc2c(=O)n1-c1ccc(NC(=O)Cc2cccnc2)cc1 ; Cc1cc(CC(=O)Nc2ccc(-n3c(C)nc4cc(F)c(F)cc4c3=O)cc2)cc(C)n1 ; Cc1nc2cc(F)c(F)cc2c(=O)n1-c1ccc(NC(=O)Cc2cc(C(F)(F)F)cc(C(F)(F)F)c2)cc1]

7. The compound for use of claim 1, and 3 to 5 or the pharmaceutical composition according to claim 2 to 5, wherein the compound is selected from the group consisting of

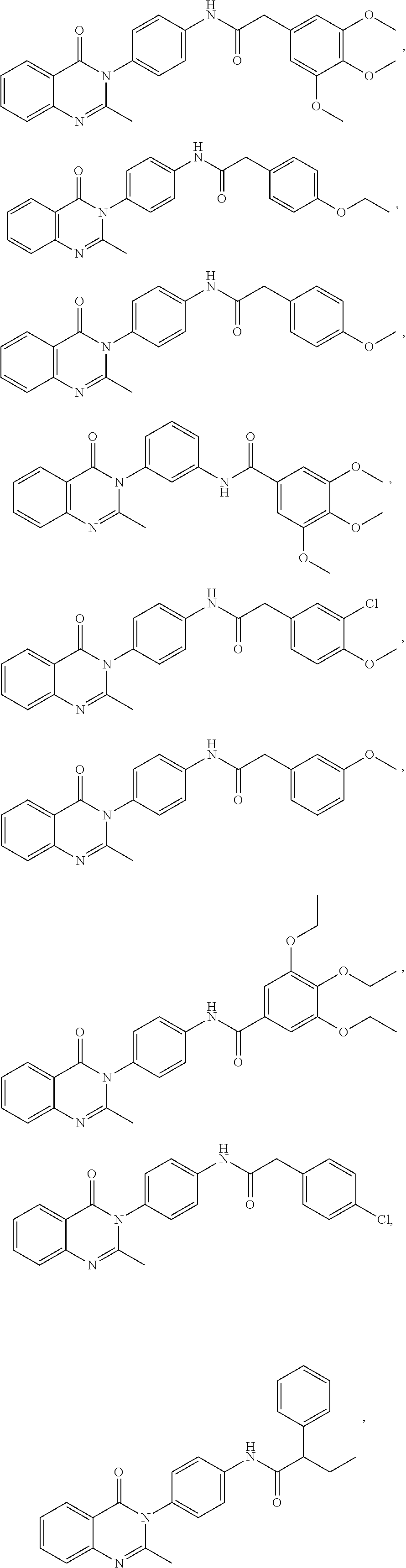

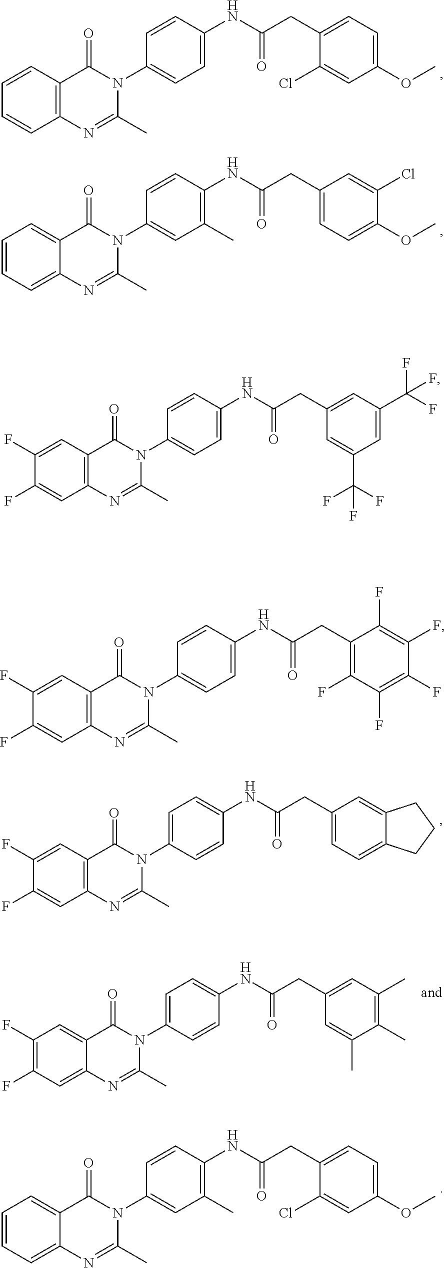

8. The compound for use of claim 1, and 3 to 5 or the pharmaceutical composition according to claim 2 to 5, wherein the compound is selected from the group consisting of

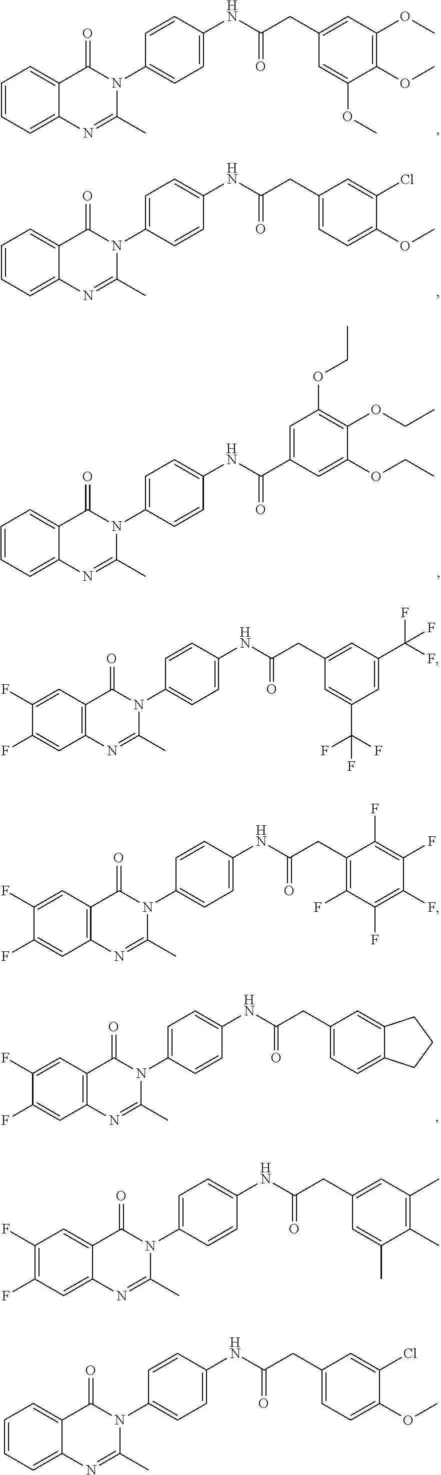

9. A compound according to formula (I), or (II) 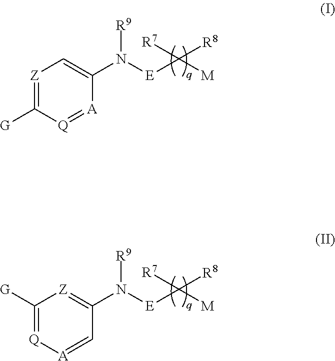
wherein G is selected from the group consisting of 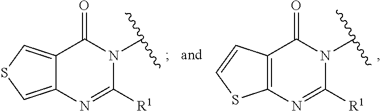
preferably 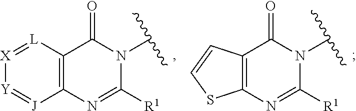
M is selected from the group consisting of 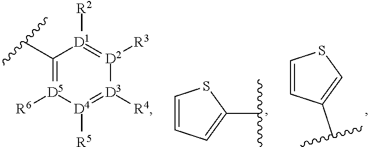
preferably 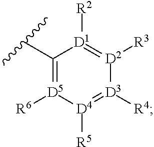
Z is selected from the group consisting of CH, C—(C₁-C₆)alkyl, and N, preferably CH;
A is selected from the group consisting of CH, C—(C₁-C₆)alkyl, and N, preferably CH;
Q is selected from the group consisting of CH, C—(C₁-C₆)alkyl, and N, preferably CH;
E is selected from the group consisting of —CO—, —SO₂—, preferably —CO—;
L is selected from the group consisting of CH, N, C—F, C—Cl, and C—(C₁-C₆)alkyl, preferably CH;
X is selected from the group consisting of CH, N, C—F, C—Cl, and C—(C₁-C₆)alkyl, preferably CH;
Y is selected from the group consisting of CH, N, C—F, C—Cl, and C—(C₁-C₆)alkyl, preferably CH;
X and Y may be part of a ring according to formula (III): 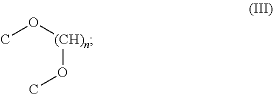
n is an integer from 1 to 3;
J is selected from the group consisting of CH, N, C—F, C—Cl, C—(C₁-C₆)alkyl, preferably CH;
D¹, D², D³, D⁴, D⁵, are independently selected from the group consisting of C and N;
R¹ is selected from the group consisting of —(C₁-C₆)alkyl, —(C₃-C₆)cycloalkyl, —CH₂OH, —CH₂F, —CHF₂, and —CF₃, preferably —(C₁-C₆)alkyl;
R² is selected from the group consisting of H, —(C₁-C₆)alkyl, CF₃, —Cl, and —F, preferably H;
R³ is selected from the group consisting of H, —(C₁-C₆)alkyl, —O(C₁-C₆)alkyl, CF₃, —Cl, and —F, preferably —O(C₁-C₆)alkyl;
R⁴ is selected from the group consisting of H, —(C₁-C₆)alkyl, —O(C₁-C₆)alkyl, CF₃, —Cl, and —F, preferably —O(C₁-C₆)alkyl;
R⁵ is selected from the group consisting of H, —(C₁-C₆)alkyl, —O(C₁-C₆)alkyl, CF₃, —Cl, and —F, preferably —O(C₁-C₆)alkyl;
R⁶ is selected from the group consisting of H, —(C₁-C₆)alkyl, CF₃, —Cl, and —F, preferably H;
two groups of R², R³, R⁴, R⁵ and R⁶ in vicinal position may be part of a ring selected from 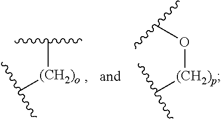
o is an integer between 1 and 4;
p is an integer between 1 and 4;
q is 0 or 1;
R⁷ and R⁸ are independently selected from the group consisting of H, and —(C₁-C₆)alkyl;
R⁷ and R⁸ may form a 3 to 5 membered cycloalkyl;
R⁹ is selected from the group consisting of H, and —(C₁-C₆)alkyl or
a pharmaceutically acceptable salt thereof,
with the provision that the compound is not selected from the group consisting of 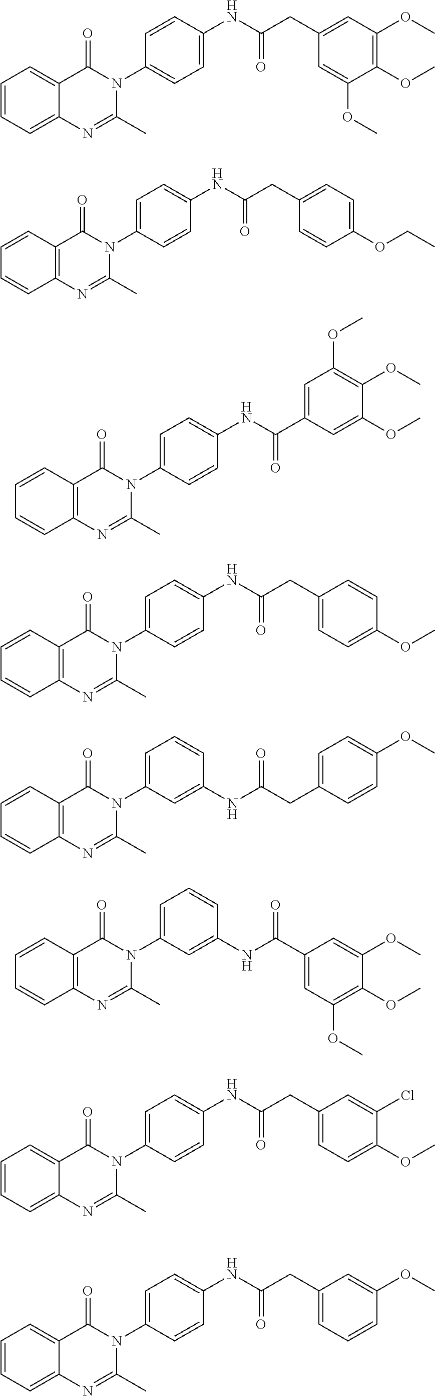 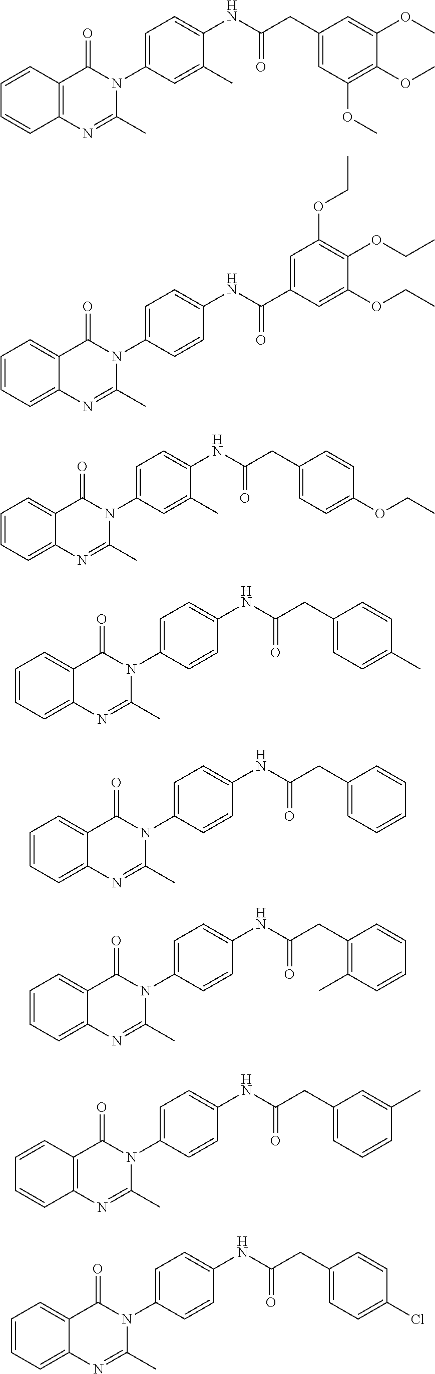 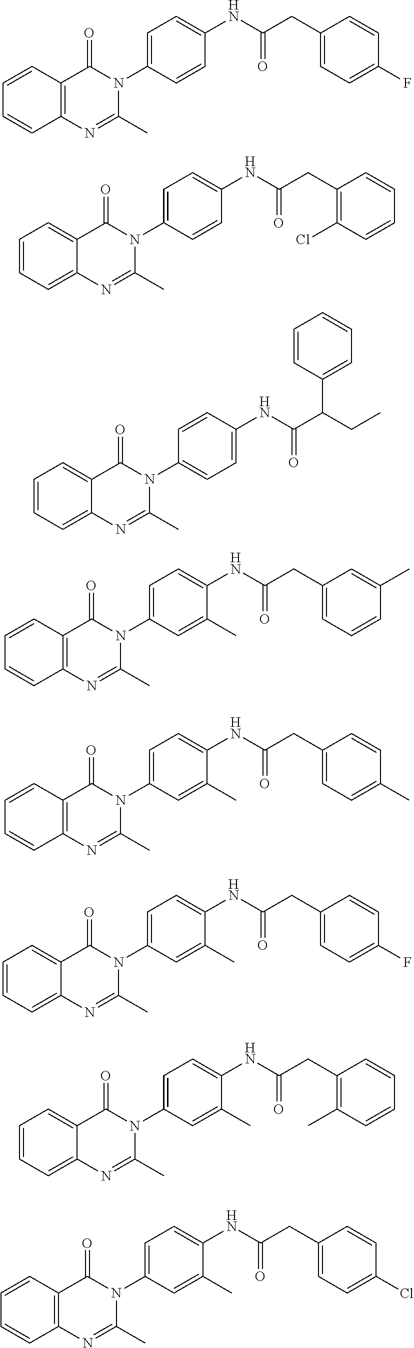 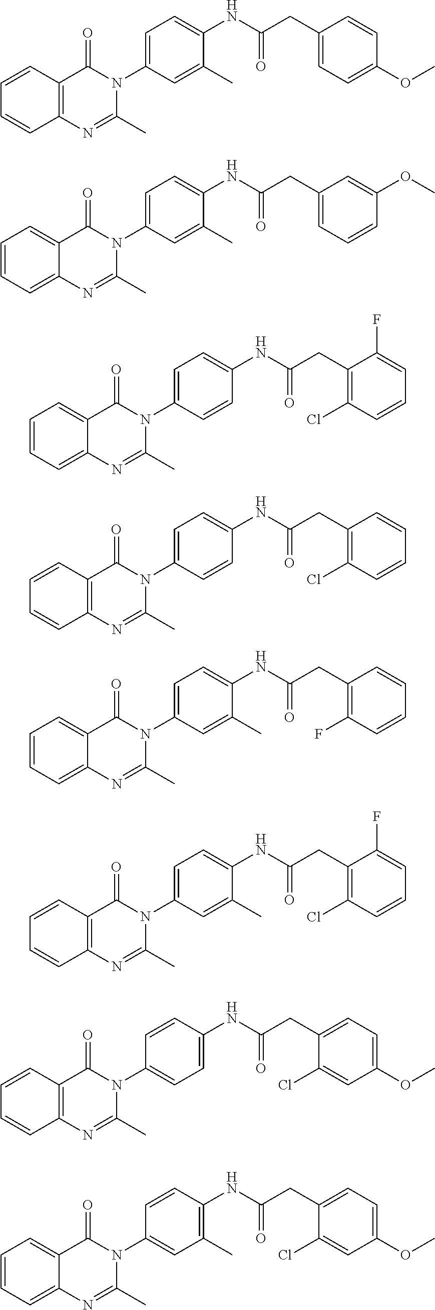 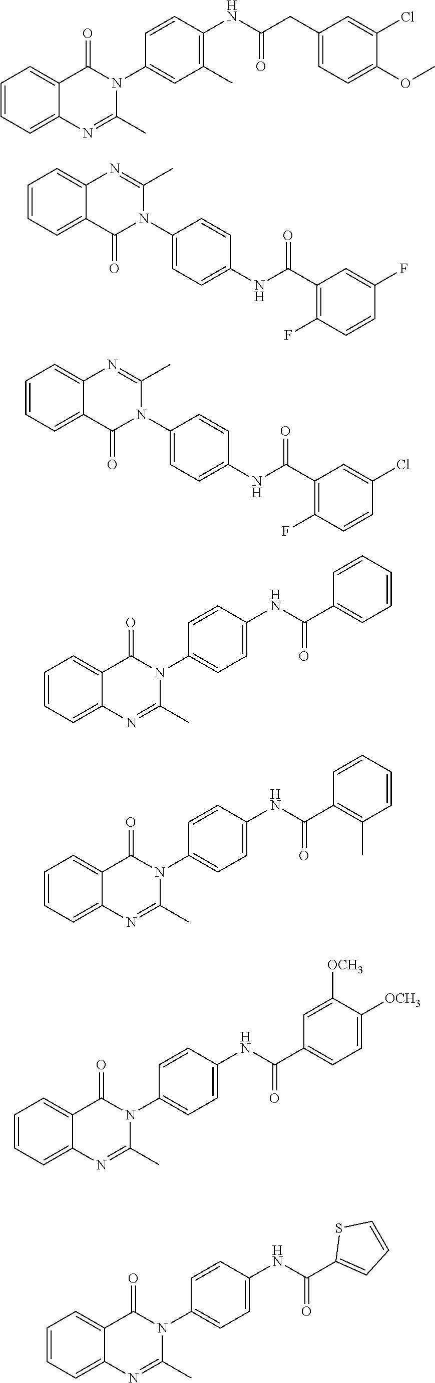 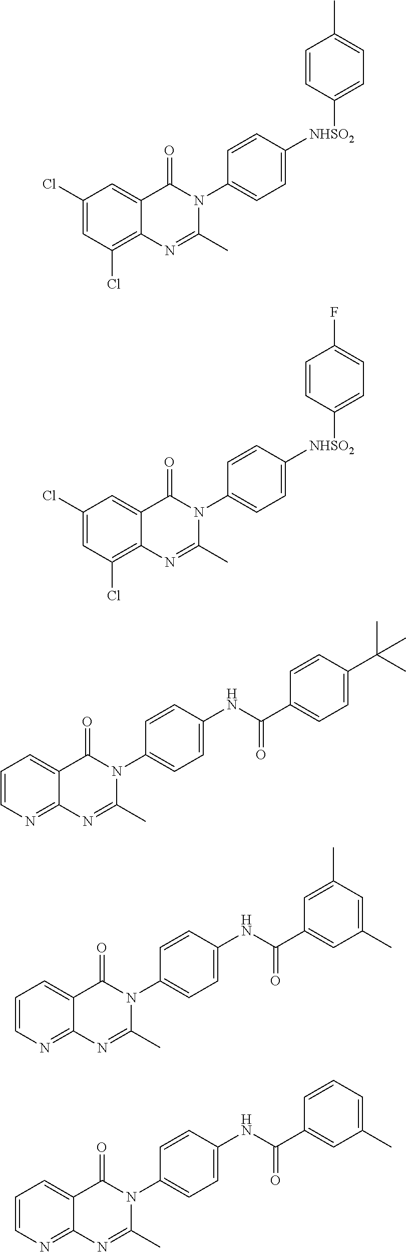 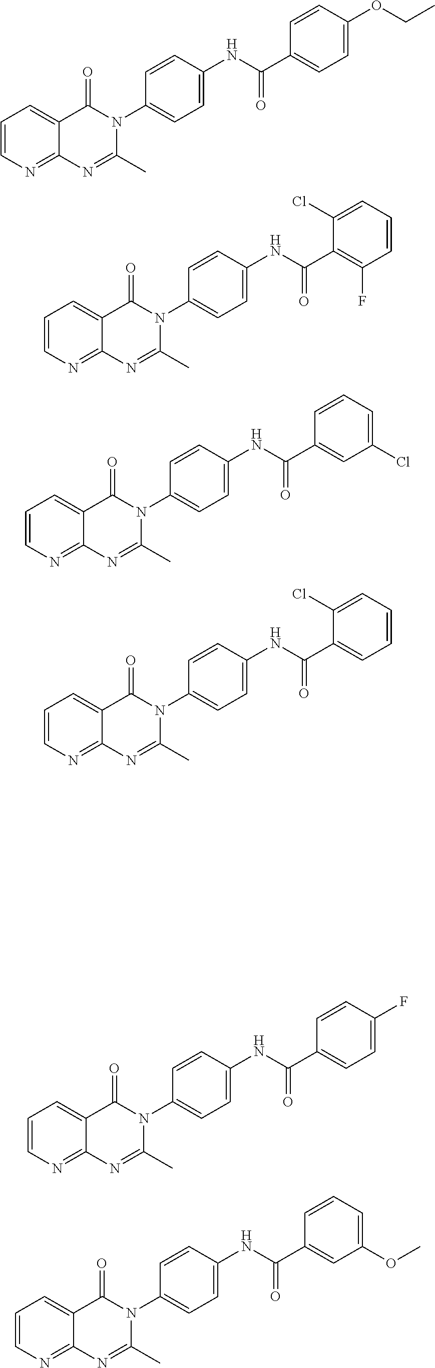 [SMILES: CCOc1ccc(C(=O)Nc2ccc(-n3c(C)nc4ncccc4c3=O)cc2)cc1 ; Cc1nc2ncccc2c(=O)n1-c1ccc(NC(=O)c2c(F)cccc2Cl)cc1 ; Cc1nc2ncccc2c(=O)n1-c1ccc(NC(=O)c2cccc(Cl)c2)cc1 ; Cc1nc2ncccc2c(=O)n1-c1ccc(NC(=O)c2ccccc2Cl)cc1 ; Cc1nc2ncccc2c(=O)n1-c1ccc(NC(=O)c2ccc(F)cc2)cc1 ; COc1cccc(C(=O)Nc2ccc(-n3c(C)nc4ncccc4c3=O)cc2)c1] 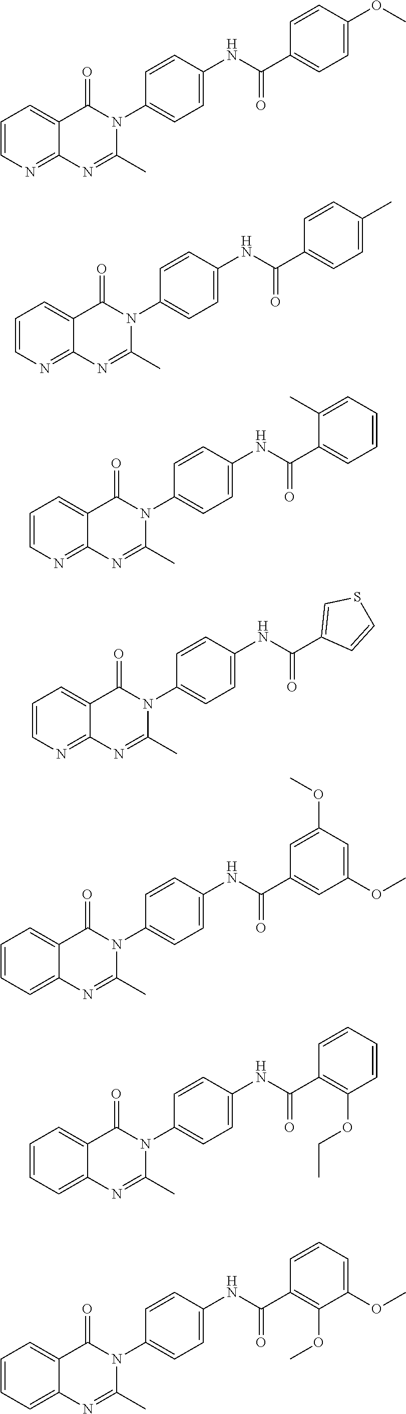 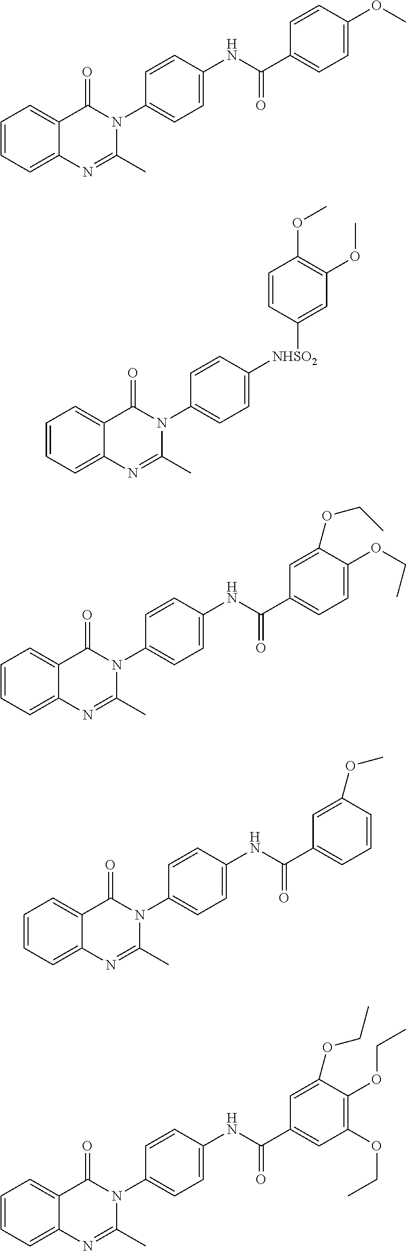
or a pharmaceutically acceptable salt thereof.

10. The compound for use according to claim 1, and 3 to 5, the pharmaceutical composition of claim 2 to 5, or the compound of claim 9, wherein in formula (I) or (II),
G is 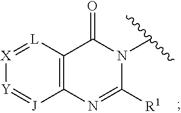
Z is CH;
A is CH;
Q is CH;
E is —CO—;
L is CH;
X is CH;
Y is preferably CH;
n is an integer from 1 to 3;
J is CH;
R¹ is —(C₁-C₆)alkyl; preferably methyl.
R² is H;
R³ is selected from the group consisting of H or —O(C₁-C₆)alkyl;
R⁴ is selected from the group consisting of H, or —O(C₁-C₆)alkyl;
R⁵ is selected from the group consisting of H, —Cl, or —O(C₁-C₆)alkyl;
R⁶ is selected from the group consisting of H or —Cl; preferably H;
o is an integer between 1 and 4;
p is an integer between 1 and 4;
q is 0 or 1;
R⁷ and R⁸ are independently selected from the group consisting of H, and —(C₁-C₆)alkyl;
R⁷ and R⁸ may form a 3 to 5 membered cycloalkyl;
R⁹ is selected from the group consisting of H, and —(C₁-C₆)alkyl.
